(19) United States
(12) Patent Application Publication
(10) Pub. No.: US 2024/0252620 A1
(43) Pub. Date: Aug. 1, 2024

(54) COMBINED AGONIST ADJUVANT FOR CORONAVIRUS VACCINE

(71) Applicants: THE REGENTS OF THE UNIVERSITY OF MICHIGAN, Ann Arbor, MI (US); ICHAN SCHOOL OF MEDICINE AT MOUNT SINAI, New York, NY (US)

(72) Inventors: Pamela Wong, Ann Arbor, MI (US); James Baker, Ann Arbor, MI (US); Sonia Jangra, New York, NY (US); Adolfo Garcia-Sastre, New York, NY (US); Michael Schotsaert, New York, NY (US)

(21) Appl. No.: 18/564,263

(22) PCT Filed: May 26, 2022
(86) PCT No.: PCT/US2022/031002

§ 371 Date: Nov. 27, 2023

Related U.S. Application Data
(60) Provisional application No. 63/194,458, filed on May 28, 2021.

(51) Int. Cl. A61K 39/215; A61K 39/00; A61P 37/04

(52) U.S. Cl. CPC A61K 39/215; A61P 37/04; A61K 2039/525; A61K 2039/5252; A61K 2039/5254; A61K 2039/5256; A61K 2039/53; A61K 2039/543; A61K 2039/55516; A61K 2039/55561; A61K 2039/55566; A61K 2039/575

(57) ABSTRACT

The disclosure is directed to compositions and methods for inducing an immune response against a coronavirus, which involve a coronavirus vaccine and an adjuvant composition. The adjuvant composition comprises a nanoemulsion, an agonist of retinoic acid-inducible gene I (RIG-I), and/or an agonist of a toll-like receptor.

### STATEMENT OF RELATED APPLICATIONS

This application claims priority to U.S. Provisional Patent Application No. 63/194,458, filed May 28, 2022, the entire contents of which are incorporated herein by reference for all purposes.

### BACKGROUND

The rapid global spread of severe acute respiratory syndrome coronavirus 2 (SARS-CoV-2) has had a devastating impact on human health and global economies. Global cases of SARS-CoV-2 infection have exceeded 100 million, and more than 2.1 million fatalities have occurred. Several promising vaccines, including RNA-based, adenovirus vectored, and inactivated viral vaccines are in the final phases of clinical testing and some have now received emergency use authorizations (EUAs) in various countries. Candidate subunit vaccines are soon to follow. However, many parameters remain to be determined for first generation vaccines, such as the duration and breadth of conferred immunity, whether or not vaccine induced immunity is sterilizing, and real-world efficacy, particularly in cohorts which traditionally display low response rates to vaccination, such as the elderly and immunocompromised. In addition, new genetic variants of SARS-CoV-2 have arisen which are reported to show higher transmissibility, increased virulence, and the potential for escape from current vaccines. Thus, it is clear that successful control of the pandemic will require vaccines which can provide not only robust and long-lasting protection, but also confer broad immunity towards these variants and potential future variants.

There remains a need for methods and compositions for inducing potent and durable immune responses against coronaviruses, such as SARS-CoV-2.

### BRIEF SUMMARY

The disclosure provides immunogenic compositions and methods of using same for inducing an immune response against a coronavirus in a subject.

In some aspects, the disclosure provides an immunogenic composition comprising one or more of: (a) a nanoemulsion; (b) an agonist of retinoic acid-inducible gene I (RIG-I) and/or an agonist of a toll-like receptor (TLR); and (c) a coronavirus vaccine.

The disclosure further provides the use of the above-described immunogenic composition in the preparation of a medicament, such as a medicament for immunizing an animal against a coronavirus.

The disclosure also provides a method of inducing an immune response in a subject, which comprises administering a therapeutically effective amount of the above-described immunogenic composition to the subject.

In other aspects, the disclosure provides a method of inducing coronavirus-specific neutralizing antibodies and/or coronavirus-specific T cell responses in a subject, which comprises administering a therapeutically effective amount of the above-described immunogenic composition to the subject.

In other aspects, the disclosure provides a method for inducing an immune response against a coronavirus in a subject, which method comprises administering to a subject in need thereof (i) a coronavirus vaccine (ii) a nanoemulsion and (iii) an agonist of retinoic acid-inducible gene I (RIG-I) and/or an agonist of a toll-like receptor (TLR).

### BRIEF DESCRIPTION OF THE DRAWING(S)

FIG. 1 shows results of an assessment of acute cytokine response in the serum by measuring IL-6 (FIG. 1A, TNF-α (FIG. 1), IL12p70 (FIG. 1C), and IFN-γ (FIG. 1D) by multiplex immunoassay at 6 and 12 h post-intranasal (IN) immunization with 10 μg of RBD only, or with 20% NE, or 20% NE/0.5 μg IVT DI.

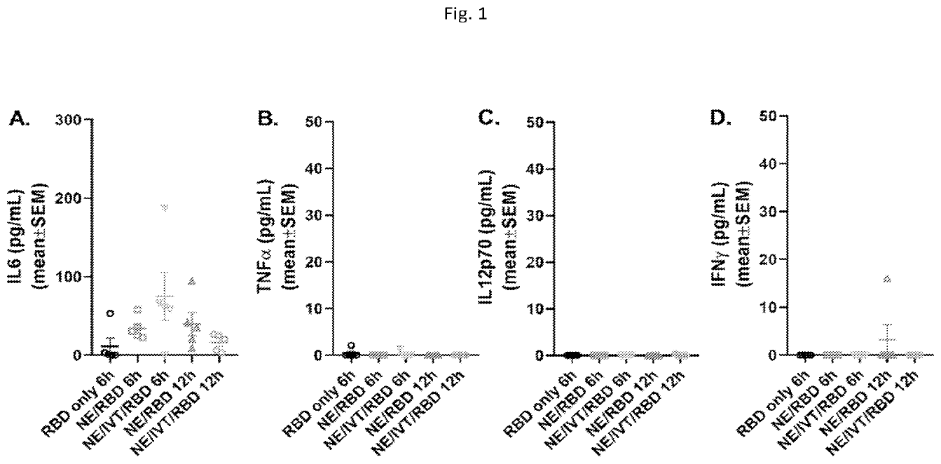

FIG. 2 shows serum SI-specific IgG measured in mice immunized IN with 15 μg S1 alone, or with 20% NE, or 20%/0.5 μg IVT DI. S1-specific IgG was measured 2 weeks after each immunization at (FIG. 2A) 2 weeks post-initial immunization (prime), (FIG. 2B) 6 weeks post-initial immunization (prime/boost), and (FIG. 2C) 10 weeks post-initial immunization (prime/boost/boost). (*p<0.05, **p<0.01 by Mann-Whitney U test). FIG. 2D shows antibody avidity for S1-specific IgG measured by NaSCN elution for a 1:1,250 dilution of serum for the 6 wk and 10 wk sera. The control group (ctrl) consisted of untreated mice.

FIG. 3 shows serum S1-specific IgG subclasses (FIG. 3A) IgG1, (FIG. 3B) IgG2b, and (FIG. 3C) IgG2c measured in mice immunized IN with 15 μg S1 alone, or with 20% NE, or 20%/0.5 μg IVT DI after the last boost immunization, 10 weeks post-initial immunization. (*p<0.05, **p<0.01 by Mann-Whitney U test).

FIG. 4 shows neutralizing antibody titers in serum from mice receiving two (wk 6) or three (week 10) immunizations determined using microneutralization assays against the wild-type (WT) SARS-CoV-2 (2019-nCoV/USA-WA1/2020) virus, pseudotyped lentivirus expressing the WT SARS-CoV-2 spike protein (Lenti-CoV2), and a mouse-adapted (MA) SARS-CoV-2. Viral neutralization was plotted as percentage inhibition of viral infection in Vero E6 cells (for WT virus and MA-virus) relative to virus only (no serum) positive controls versus the inverse serum dilution. The titer at which 50% inhibition of infection was achieved (IC50) was determined for the (FIGS. 4A, 4B) WT virus and the (FIGS. 4D, 4E) MA virus. FIG. 4C is a graph confirming the results for the same week 10 serum samples using the Lenti-CoV2 pseudovirus expressing firefly luciferase with HEK-293T cells expressing hACE2. Microneutralization titers using the Lenti-CoV2 were determined by detecting viral infection by measuring luminescence (*p<0.05, **p<0.01 by Mann-Whitney U test). Pretreatment (pre) sera were obtained from the same set of mice before immunizations.

FIG. 5 shows protection offered by passive transfer of serum from vaccinated mice against heterologous challenge with MA-SARS-CoV-2. Naïve C57Bl/6 mice (n=3-4/group) each received 150 μL of pooled serum through the intraperitoneal route from donor mice given three IN immunizations of S1, NE/S1, or NE/IVT/S1. Two hours after serum transfer, mice were challenged IN with 10⁴ PFU of MA-SARS-CoV-2. FIG. 5A shows body weight loss measured over three days, and FIG. 5B shows lung virus titers at 3 d.p.i. as determined in homogenate from one lobe of the isolated lungs by plaque assay (solid symbols). Passive transfer/challenge was repeated for the PBS control and S1 sera for verification, and virus titers were measured in whole lung in the replicated experiment (open square symbols). (*p<0.05, **p<0.01 by Mann-Whitney U test assessed for half lung data points).

FIG. 6 shows antigen recall response assessed in splenocytes isolated from mice immunized IN with S1 alone, or with NE, or NE/IVT DI after the final boost immunization (10 weeks post-initial immunization). Splenocytes were stimulated ex vivo with 5 μg of recombinant S1 for 72 h, and levels of secreted cytokines (FIG. 6A) IFNγ, (FIG. 6B) IL2, (FIG. 6C) IP10, (FIG. 6D) TNFα, (FIG. 6E) IL6, (FIG. 6F) IL17A, (FIG. 6G) IL4, (FIG. 6H) IL5, (FIG. 6I) IL13 were measured in the supernatant relative to unstimulated cells by multiplex immunoassay. An unvaccinated control was included for comparison. (n=5/grp; *p<0.05, **p<0.01 by Mann-Whitney U test).

FIG. 7 shows antigen recall response assessed in lymphocytes from draining lymph nodes (cLN) isolated from mice immunized IN with S1 alone, or with NE, or NE/IVT DI after the final boost immunization (10 weeks post-initial immunization). Cells were stimulated ex vivo with 5 μg of recombinant S1 for 72 h, and levels of secreted cytokines (FIG. 7A) IFNγ, (FIG. 7B) IL2, (FIG. 7C) IP10, (FIG. 7D) TNFα, (FIG. 7E) IL6, (FIG. 7F) IL17A, (FIG. 7G) IL4, (FIG. 7H) IL5, (FIG. 7I) IL13 were measured in the supernatant relative to unstimulated cells by multiplex immunoassay. An unvaccinated control was included for comparison. (n=5/grp; *p<0.05, **p<0.01 by Mann-Whitney U test).

FIG. 8 shows antigen recall response assessed in lymphocytes from spleen (FIG. 8A) and cLN (FIG. 8B) isolated from mice immunized IM with 10 μg SARS-CoV-2 RBD alone, or with 50% Addavax (a licensed emulsion based adjuvant) in a volume of 50 μL according to a prime/boost/boost schedule (at a 4 wk interval). Cells were stimulated ex vivo with 5 μg of recombinant RBD for 72 h, and levels of secreted IL-5 were measured in the supernatant relative to unstimulated cells by multiplex immunoassay. (n=5/grp; *p<0.05, **p<0.01 by Mann-Whitney U test).

FIG. 9 shows the mucosal immune response assessed in immunized mice by measuring S1-specific IgA in bronchial alveolar lavage after prime/boost/boost immunizations (10 weeks post-initial immunization) as measured by ELISA. Absorbance values at 405 nm are shown after development with an alkaline-phosphatase conjugated secondary antibody with a pNPP substrate.

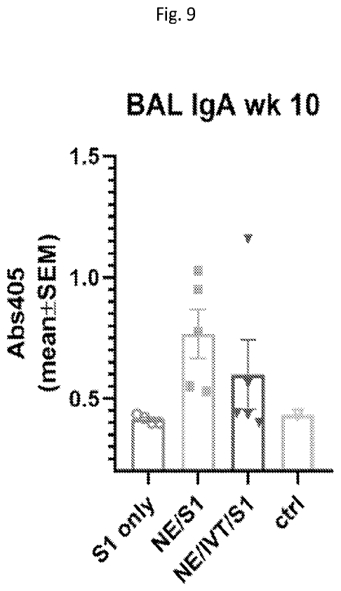

FIG. 10 shows RBD-specific humoral and mucosal immune responses induced in aged versus young mice. RBD-specific IgG titers were measured in sera two weeks after each immunization after (A) prime (wk2) (B) prime/boost (wk 6) and (C) prime/boost/boost (wk 10). Serum RBD-specific IgG subclass titers, (D) IgG1, (E) IgG2b, (F) IgG2c, were measured at wk10. Titers are shown as mean±SEM (n=5-10/grp) (G) Mucosal RBD-specific IgA was measured in BAL at wk10 by ELISA. (*p<0.05, **p<0.01 by Mann-Whitney U test).

FIG. 11 shows serum viral neutralizing antibody titers in aged vs. young, immunized mice. Neutralizing antibody titers measured in sera from mice immunized three times IN with 10 or 20 μg RBD with PBS, NE, or NE/IVT, or IM with RBD with Addavax using lentivirus PSVs expressing the SARS-CoV-2 S protein derived from (A) WT, (B) B.1.617.2, (C) B.1.351, (D) B.1.1.529 viral variants. Titers are shown as mean±SEM (n=5/grp) (*p<0.05, **p<0.01 by Mann-Whitney U test).

FIG. 12 shows passive transfer of serum from young and aged immunized mice into naïve mice provides protection from challenge with MA-SARS-CoV-2 and offers some cross-protection to B.1.351. (A) Sera from young or aged mice given two IN immunizations with 20 μg RBD with PBS, NE, or NE/IVT were pooled at wk 6, and 50 μL of the pooled serum was transferred IP into each naïve mouse 2 h prior to challenge IN with 10⁴ pfu MA-SARS-CoV-2. (B) Sera from young or aged mice given three IN immunizations with 20 μg RBD with PBS, NE, or NE/IVT were pooled at wk 10, and 110 μL of the pooled serum was transferred IP into each naïve mouse 2 h prior to challenge IN with 5×10³ pfu B.1.351. Lung viral titers are shown as mean±SEM (n=5/grp) (*p<0.05, **p<0.01 by Mann-Whitney U test).

FIG. 13 shows antigen recall response as assessed in splenocytes from aged vs. young, immunized mice. Levels of secreted cytokines were measured in the cell supernatant by multiplex immunoassay and compared to unstimulated cells for (A) IFN-γ, (B) IL-2, (C) IP-10, (D) TNF-α, (E) IL-4, (F) IL-5, (G) IL-13, (H) IL-6, (I) IL-17A, and (J) IL-10. (n=5/grp; *p<0.05, **p<0.01 by Mann-Whitney U test).

FIG. 14 shows antigen recall response as assessed in cLN from aged vs. young, immunized mice. Levels of secreted cytokines were measured in the cell supernatant by multiplex immunoassay relative to unstimulated cells for (A) IFN-γ, (B) IL-2, (C) IP-10, (D) TNF-α, (E) IL-4, (F) IL-5, (G) IL-13, (H) IL-6, (I) IL-17A, and (J) IL-10. (n=5/grp; *p<0.05, **p<0.01 by Mann-Whitney U test).

FIG. 15 shows longevity of humoral immune responses induced by IN immunization. (A) Serum RBD-specific IgG titers were followed for 25 weeks after the last boost. Neutralizing antibody titers in sera from the same immunized mice were compared between (B) NE/10 RBD, or (C) NE/IVT/10 RBD groups 6 weeks after the last boost immunization or 25 weeks after the last boost immunization. (n=5/grp; *p<0.05, **p<0.01 by Mann-Whitney U test).

FIG. 16 shows longevity of cellular immune responses induced by IN immunization. Levels of secreted cytokines were measured in the cell supernatant by multiplex immunoassay relative to unstimulated cells for (A) IFN-γ, (B) IL-2, (C) IP-10, (D) TNF-α, (E) IL-4, (F) IL-5, (G) IL-13, (H) IL-6, (I) IL-17A, and (J) IL-10. (n=5/grp; *p<0.05, **p<0.01 by Mann-Whitney U test)

### DEFINITIONS

To facilitate an understanding of the present technology, a number of terms and phrases are defined below. Additional definitions are set forth throughout the detailed description.

The terms “host” or “subject,” as used herein, refer to an individual to be treated by (e.g., administered (e.g., injectably administered)) compositions and methods of the present disclosure. Subjects include, but are not limited to, mammals (e.g., murines, simians, equines, bovines, porcines, canines, felines, and the like), and most preferably includes humans. In the context of the disclosure, the term “subject” generally refers to an individual who will be administered (e.g., injectably and/or intranasal administered) or who has been administered one or more compositions of the present disclosure (e.g., a coronavirus vaccine and a composition comprising a nanoemulsion and an agonist of retinoic acid-inducible gene I (RIG-I)).

In some embodiments, the subject is at elevated risk for infection (e.g., by a coronavirus). In some embodiments, the subject may have a healthy or normal immune system. In some embodiments, the subject is one that has a greater than normal risk of being exposed to a pathogen (e.g., a coronavirus). In some embodiments, the subject is a soldier, an emergency responder or other subject that has a higher than normal risk of being exposed to a pathogen (e.g., a coronavirus).

As used herein, the terms “at risk for infection” and “at risk for disease” refer to a subject that is predisposed to experiencing a particular infection or disease (e.g., a coronavirus). This predisposition may be genetic, or due to other factors (e.g., age, immunosuppression, compromised immune system, immunodeficiency, environmental conditions, exposures to detrimental compounds present in the environment, etc.). Thus, it is not intended that embodiments of the present disclosure be limited to any particular risk (e.g., a subject may be “at risk for disease” simply by being exposed to and interacting with other people), nor is it intended that embodiments of the present disclosure be limited to any particular disease.

As used herein, the term “sample” is used in its broadest sense and encompasses materials obtained from any source. As used herein, the term “sample” is used to refer to materials obtained from a biological source, for example, obtained from animals (including humans), and encompasses any fluids, solids, and/or tissues. In particular embodiments of the present disclosure, biological samples include blood and blood products such as plasma, serum and the like. However, these examples are not to be construed as limiting the types of samples that find use with the present disclosure.

The term “emulsion,” as used herein, includes classic oil-in-water or water in oil dispersions or droplets, as well as other lipid structures that can form as a result of hydrophobic forces that drive apolar residues (e.g., long hydrocarbon chains) away from water and drive polar head groups toward water, when a water immiscible oily phase is mixed with an aqueous phase. These other lipid structures include, but are not limited to, unilamellar, paucilamellar, and multilamellar lipid vesicles, micelles, and lamellar phases. Similarly, the term “nanoemulsion,” as used herein, refers to oil-in-water dispersions comprising small lipid structures. For example, in some embodiments, the nanoemulsions comprise an oil phase having droplets with a mean particle size of approximately 0.1 to 5 microns (e.g., about 100, 150, 200, 250, 300, 350, 400, 450, 500 nm or larger in diameter), although smaller and larger particle sizes are contemplated. The terms “emulsion” and “nanoemulsion” and “NE” may be used interchangeably herein to refer to the nanoemulsions of the present disclosure.

The term “surfactant” refers to any molecule having both a polar head group, which energetically prefers solvation by water, and a hydrophobic tail that is not well solvated by water. The term “cationic surfactant” refers to a surfactant with a cationic head group. The term “anionic surfactant” refers to a surfactant with an anionic head group.

As used herein, the term “adjuvant” refers to any substance that can stimulate an immune response (e.g., a mucosal immune response). Some adjuvants can cause activation of a cell of the immune system (e.g., an adjuvant can cause an immune cell to produce and secrete a cytokine). Examples of adjuvants that can cause activation of a cell of the immune system include, but are not limited to, saponins purified from the bark of the Q. saponaria tree, such as QS21 (a glycolipid that elutes in the 21st peak with HPLC fractionation; Aquila Biopharmaceuticals, Inc., Worcester, Mass.); poly(di(carboxylatophenoxy)phosphazene (PCPP polymer; Virus Research Institute, USA); derivatives of lipopolysaccharides such as monophosphoryl lipid A (MPL; Ribi ImmunoChem Research, Inc., Hamilton, Mont.), muramyl dipeptide (MDP; Ribi) and threonyl-muramyl dipeptide (t-MDP; Ribi); OM-174 (a glucosamine disaccharide related to lipid A; OM Pharma SA, Meyrin, Switzerland); and Leishmania elongation factor (a purified Leishmania protein; Corixa Corporation, Seattle, Wash.). Traditional adjuvants are well known in the art and include, for example, aluminum phosphate or hydroxide salts (“alum”).

A used herein, the term “immune response” and grammatical equivalents thereof refer to a response by the immune system of a subject. For example, immune responses include, but are not limited to, a detectable alteration (e.g., increase) in Toll-like receptor (TLR) activation, lymphokine (e.g., cytokine (e.g., Th1 or Th2 type cytokines) or chemokine) expression and/or secretion, macrophage activation, dendritic cell activation, T cell activation (e.g., CD4+ or CD8+ T cells), NK cell activation, and/or B cell activation (e.g., antibody generation and/or secretion). Additional examples of immune responses include binding of an immunogen (e.g., antigen (e.g., immunogenic polypeptide)) to an MHC molecule and inducing a cytotoxic T lymphocyte (“CTL”) response, inducing a B cell response (e.g., antibody production), and/or T-helper lymphocyte response, and/or a delayed type hypersensitivity (DTH) response against the antigen from which the immunogenic polypeptide is derived, expansion (e.g., growth of a population of cells) of cells of the immune system (e.g., T cells, B cells (e.g., of any stage of development (e.g., plasma cells), and increased processing and presentation of antigen by antigen presenting cells. An immune response may be to immunogens that the subject's immune system recognizes as foreign (e.g., non-self antigens from microorganisms (e.g., pathogens), or self-antigens recognized as foreign). Thus, it is to be understood that, as used herein, “immune response” refers to any type of immune response, including, but not limited to, innate immune responses (e.g., activation of Toll receptor signaling cascade), cell-mediated immune responses (e.g., responses mediated by T cells (e.g., antigen-specific T cells) and non-specific cells of the immune system), and humoral immune responses (e.g., responses mediated by B cells (e.g., via generation and secretion of antibodies into the plasma, lymph, and/or tissue fluids). The term “immune response” is meant to encompass all aspects of the capability of a subject's immune system to respond to antigens and/or immunogens (e.g., both the initial response to an immunogen (e.g., a pathogen) as well as acquired (e.g., memory) responses that are a result of an adaptive immune response).

As used herein, the terms “toll receptors” and “TLRs” refer to a class of receptors (e.g., TLR1, TLR2, TLR3, TLR4, TLR5, TLR6, TLR7, TLR8, TLR9, TLR10, TLR 11) that recognize special patterns of pathogens, termed pathogen-associated molecular patterns (see, e.g., Janeway and Medzhitov, (2002) Annu. Rev. Immunol., 20: 197-216). These receptors are expressed in innate immune cells (e.g., neutrophils, monocytes, macrophages, dendritic cells) and in other types of cells such as endothelial cells. Their ligands include bacterial products such as LPS, peptidoglycans, and lipopeptides. TLRs are receptors that bind to exogenous ligands and mediate innate immune responses leading to the elimination of invading microbes. The TLR-triggered signaling pathway leads to activation of transcription factors including NFκB, which is important for the induced expression of proinflammatory cytokines and chemokines. TLRs also interact with each other. For example, TLR2 can form functional heterodimers with TLR1 or TLR6. The TLR2/1 dimer has a different ligand binding profile than the TLR2/6 dimer (Ozinsky et al., PNAS, 97(25): 13766-13771 (2000)). In some embodiments, a nanoemulsion adjuvant activates cell signaling through a TLR (e.g., TLR2, TLR3, and/or TLR4). Thus, methods described herein include a nanoemulsion adjuvant composition combined with one or more immunogens (e.g., a vaccine, protein antigens, or other antigen described herein)) that when administered to a subject, activates one or more TLRs and stimulates an immune response (e.g., innate and/or adaptive/acquired immune response) in a subject. Such an adjuvant can activate TLRs (e.g., TLR2, TLR3, and/or TLR4) by, for example, interacting with TLRs (e.g., NE adjuvant binding to TLRs) or activating any downstream cellular pathway that occurs upon binding of a ligand to a TLR. NE adjuvants described herein that activate TLRs can also enhance the availability or accessibility of any endogenous or naturally occurring ligand of TLRs. A NE adjuvant that activates one or more TLRs can alter transcription of genes, increase translation of mRNA, or increase the activity of proteins that are involved in mediating TLR cellular processes. For example, NE adjuvants described herein that activate one or more TLRs (e.g., TLR2, TLR3, and/or TLR4) can induce expression of one or more cytokines (e.g., IL-8, IL-12p40, and/or IL-23).

As used herein, the term “immunity” refers to protection from disease (e.g., preventing or attenuating (e.g., suppression) of a sign, symptom or condition of the disease) upon exposure to a microorganism (e.g., pathogen) capable of causing the disease. Immunity can be innate (e.g., non-adaptive (e.g., non-acquired) immune responses that exist in the absence of a previous exposure to an antigen) and/or acquired/adaptive (e.g., immune responses that are mediated by B and T cells following a previous exposure to antigen (e.g., that exhibit increased specificity and reactivity to the antigen)).

As used herein, the term “antibody” refers to an immunoglobulin molecule that is typically composed of two identical pairs of polypeptide chains, each pair having one “light” (L) chain and one “heavy” (H) chain. Human light chains are classified as kappa and lambda light chains. Heavy chains are classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 3 or more amino acids. Each heavy chain is comprised of a heavy chain variable region (abbreviated herein as HCVR or V_{H}) and a heavy chain constant region. The heavy chain constant region is comprised of three domains, C_{H1}, C_{H2} and C_{H3}. Each light chain is comprised of a light chain variable region (abbreviated herein as LCVR or V_{L}) and a light chain constant region. The light chain constant region is comprised of one domain, CL. The constant regions of the antibodies may mediate the binding of the immunoglobulin to host tissues or factors, including various cells of the immune system (e.g., effector cells) and the first component (C1q) of the classical complement system. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of each heavy/light chain pair (V_{H} and V_{L}), respectively, form the antibody binding site. The term “antibody” encompasses an antibody that is part of an antibody multimer (a multimeric form of antibodies), such as dimers, trimers, or higher-order multimers of monomeric antibodies. It also encompasses an antibody that is linked or attached to, or otherwise physically or functionally associated with, a non-antibody moiety. Further, the term “antibody” is not limited by any particular method of producing the antibody. For example, it includes, inter alia, recombinant antibodies, synthetic antibodies, monoclonal antibodies, polyclonal antibodies, bi-specific antibodies, and multi-specific antibodies.

The terms “fragment of an antibody,” “antibody fragment,” and “functional fragment of an antibody” are used interchangeably herein to mean one or more fragments of an antibody that retain the ability to specifically bind to an antigen (see, generally, Holliger et al., Nat. Biotech., 23(9): 1126-1129 (2005)). An antibody fragment desirably comprises, for example, one or more CDRs, the variable region (or portions thereof), the constant region (or portions thereof), or combinations thereof. Examples of antibody fragments include, but are not limited to, (i) a Fab fragment, which is a monovalent fragment consisting of the V_{L}, V_{H}, C_{L}, and C_{H}1 domains, (ii) a F(a′)₂ fragment, which is a bivalent fragment comprising two Fab fragments linked by a disulfide bridge at the hinge region, (iii) a Fv fragment consisting of the V_{L} and V_{H} domains of a single arm of an antibody, (iv) a Fab′ fragment, which results from breaking the disulfide bridge of an F(ab′)₂ fragment using mild reducing conditions, (v) a disulfide-stabilized Fv fragment (dsFv), and (vi) a domain antibody (dAb), which is an antibody single variable region domain (V_{H} or V_{L}) polypeptide that specifically binds antigen.

As used herein, the term “antibody derivative” or “derivative” of an antibody refers to a molecule that is capable of binding to the same antigen that the antibody from which it is derived binds to and comprises an amino acid sequence that is the same or similar to the antibody linked to an additional molecular entity. The amino acid sequence of the antibody that is contained in the antibody derivative may be the full-length antibody, or may be any portion or portions of a full-length antibody. The additional molecular entity may be a chemical or biological molecule. Examples of additional molecular entities include chemical groups, amino acids, peptides, proteins (such as enzymes, antibodies), and chemical compounds. The additional molecular entity may have any utility, such as for use as a detection agent, label, marker, pharmaceutical or therapeutic agent. The amino acid sequence of an antibody may be attached or linked to the additional entity by chemical coupling, genetic fusion, noncovalent association or otherwise. The term “antibody derivative” also encompasses chimeric antibodies, humanized antibodies, and molecules that are derived from modifications of the amino acid sequences of an antibody, such as conservation amino acid substitutions, additions, and insertions.

In the context of the present disclosure, a “neutralizing antibody” is an antibody that binds to a virus (e.g., a coronavirus) and interferes with the virus' ability to infect a host cell.

As used herein, the term “an amount effective to induce an immune response” (e.g., of a composition for inducing an immune response), refers to the dosage level required (e.g., when administered to a subject) to stimulate, generate and/or elicit an immune response in the subject. An effective amount can be administered in one or more administrations (e.g., via the same or different route), applications or dosages and is not intended to be limited to a particular formulation or administration route.

As used herein, the term “under conditions such that said subject generates an immune response” refers to any qualitative or quantitative induction, generation, and/or stimulation of an immune response (e.g., innate or acquired).

As used herein, the terms “immunogen” and “antigen” are used interchangeably to refer to an agent (e.g., a microorganism (e.g., bacterium, virus, or fungus) and/or portion or component thereof (e.g., protein, glycoprotein, lipoprotein, peptide, glycopeptide, lipopeptide, toxoid, carbohydrate, tumor-specific antigen, etc.)) that is capable of eliciting an immune response in a subject. In preferred embodiments, immunogens elicit immunity against the immunogen (e.g., a coronavirus or a coronavirus antigen) when administered in combination with a nanoemulsion adjuvant formulation of the disclosure comprising one or more antigens/immunogens (e.g., a coronavirus antigen) together with an adjuvant formulation comprising an emulsion delivery system formulated for administration, e.g., via injectable route (e.g., intradermal, intramuscular, subcutaneously, etc.), mucosal route (e.g., nasally or vaginally), or other route, to a subject.

By “epitope” is meant a sequence of an antigen that is recognized by an antibody or an antigen receptor. Epitopes also are referred to in the art as “antigenic determinants.” In certain embodiments, an epitope is a region of an antigen that is specifically bound by an antibody or a T cell receptor. In certain embodiments, an epitope may include chemically active surface groupings of molecules such as amino acids, sugar side chains, phosphoryl, or sulfonyl groups. In certain embodiments, an epitope may have specific three-dimensional structural characteristics (e.g., a “conformational” epitope) and/or specific charge characteristics. The immunogen or antigen can be a protein or peptide of viral, bacterial, parasitic, fungal, protozoan, prion, cellular, or extracellular origin, which provokes an immune response in a mammal, preferably leading to protective immunity. An immunogen or antigen also may be based on one or more antigenic components of a particular organism and can be generated using recombinant DNA technology.

“Nasal application”, as used herein, means applied through the nose into the nasal or sinus passages or both. The application may, for example, be done by drops, sprays, mists, coatings or mixtures thereof applied to the nasal and sinus passages.

A “portion” of a nucleic acid sequence comprises at least ten nucleotides (e.g., about 10 to about 5000 nucleotides). Preferably, a “portion” of a nucleic acid sequence comprises 10 or more (e.g., 15 or more, 20 or more, 25 or more, 30 or more, 35 or more, 40 or more, 45 or more, 50 or more, or 100 or more) nucleotides, but less than 5,000 (e.g., 4900 or less, 4000 or less, 3000 or less, 2000 or less, 1000 or less, 800 or less, 500 or less, 300 or less, or 100 or less) nucleotides. Preferably, a portion of a nucleic acid sequence is about 10 to about 3500 nucleotides (e.g., about 10, 20, 30, 50, 100, 300, 500, 700, 1000, 1500, 2000, 2500, or 3000 nucleotides), about 10 to about 1000 nucleotides (e.g., about 25, 55, 125, 325, 525, 725, or 925 nucleotides), or about 10 to about 500 nucleotides (e.g., about 15, 30, 40, 50, 60, 70, 80, 90, 150, 175, 250, 275, 350, 375, 450, 475, 480, 490, 495, or 499 nucleotides), or a range defined by any two of the foregoing values. More preferably, a “portion” of a nucleic acid sequence comprises no more than about 3200 nucleotides (e.g., about 10 to about 3200 nucleotides, about 10 to about 3000 nucleotides, or about 30 to about 500 nucleotides, or a range defined by any two of the foregoing values).

A “portion” of an amino acid sequence comprises at least three amino acids (e.g., about 3 to about 1,200 amino acids). Preferably, a “portion” of an amino acid sequence comprises 3 or more (e.g., 5 or more, 10 or more, 15 or more, 20 or more, 25 or more, 30 or more, 40 or more, or 50 or more) amino acids, but less than 1,200 (e.g., 1,000 or less, 800 or less, 700 or less, 600 or less, 500 or less, 400 or less, 300 or less, 200 or less, or 100 or less) amino acids. Preferably, a portion of an amino acid sequence is about 3 to about 500 amino acids (e.g., about 10, 100, 200, 300, 400, or 500 amino acids), about 3 to about 300 amino acids (e.g., about 20, 50, 75, 95, 150, 175, or 200 amino acids), or about 3 to about 100 amino acids (e.g., about 15, 25, 35, 40, 45, 60, 65, 70, 80, 85, 90, 95, or 99 amino acids), or a range defined by any two of the foregoing values. More preferably, a “portion” of an amino acid sequence comprises no more than about 500 amino acids (e.g., about 3 to about 400 amino acids, about 10 to about 250 amino acids, or about 50 to about 100 amino acids, or a range defined by any two of the foregoing values).

The term “vaccine,” as used herein, refers to a biological preparation that stimulates a subject's immune system against a particular infectious agent and provides active acquired immunity to a particular infectious disease. A vaccine typically contains an agent that resembles a disease-causing microorganism and is often made from weakened or killed forms of the microbe, its toxins, or one of its surface proteins. The agent stimulates a subject's immune system to recognize the agent as a threat, destroy it, and to further recognize and destroy any of the microorganisms associated with that agent that it may encounter in the future. Vaccines can be prophylactic (to prevent or ameliorate the effects of a future infection by a pathogen), or therapeutic (to ameliorate a disease that has already occurred, such as cancer). There are several types of vaccines known and used in the art, including, for example, inactivated virus vaccines, live-attenuated virus vaccines, messenger RNA (mRNA) vaccines, subunit vaccines, recombinant vaccines, polysaccharide vaccines, conjugate vaccines, toxoid vaccines, and viral vector vaccines. The administration of vaccines is referred to as “vaccination.”

### DETAILED DESCRIPTION

The present disclosure provides compositions and methods for inducing an immune response against a coronavirus in a subject. Coronaviruses are named for the crown-like spikes on their surface. There are four main sub-groupings of coronaviruses, known as alpha, beta, gamma, and delta. Human coronaviruses were first identified in the mid-1960s. Seven coronaviruses have been identified that can infect humans: 229E (alpha coronavirus;) NL63 (alpha coronavirus); OC43 (beta coronavirus); HKU1 (beta coronavirus); MERS-CoV (the beta coronavirus that causes Middle East Respiratory Syndrome, or MERS); SARS-CoV (the beta coronavirus that causes severe acute respiratory syndrome, or SARS); and SARS-CoV-2 (the novel coronavirus that causes coronavirus disease 2019, or COVID-19). Coronaviruses are a large family of viruses that are common in people and many different species of animals, including camels, cattle, cats, and bats. Rarely, animal coronaviruses can infect people and then spread between people such as with MERS-CoV, SARS-CoV, and SARS-CoV-2 (COVID-19). The SARS-CoV-2 virus is a betacoronavirus, like MERS-CoV and SARS-CoV. All three of these viruses have their origins in bats. MERS-CoV and SARS-CoV have been known to cause severe illness in people. The complete clinical picture with regard to COVID-19 is not fully understood. Reported illnesses have ranged from mild to severe, including illness resulting in death. Older people and people with certain underlying health conditions like heart disease, lung disease and diabetes, for example, seem to be at greater risk of serious illness.

In some embodiments, the disclosed compositions and methods induce an immune response against SARS-CoV-2. SARS-CoV-2 is a monopartite, single-stranded, and positive-sense RNA virus with a genome size of 29,903 nucleotides, making it the second-largest known RNA genome. The virus genome consists of two untranslated regions (UTRs) at the 5′ and 3′ ends and 11 open reading frames (ORFs) that encode 27 proteins. The first ORF (ORF1/ab) constitutes about two-thirds of the virus genome, encoding 16 non-structural proteins (NSPs), while the remaining third of the genome encodes four structural proteins and at least six accessory proteins. The structural proteins are spike glycoprotein (S), membrane protein (M), envelope protein (E), and nucleocapsid protein (N), while the accessory proteins are orf3a, orf6, orf7a, orf7b, orf8, and orf10 (Wu et al., Cell Host Microbe, 27: 325-328 (2020); Chan et al., Emerg. Microbes Infect., 9: 221-236 (2020); Chen et al., Lancet, 395: 507-513 (2020); and Ceraolo, C.; Giorgi, F. M, J. Med. Virol., 92, 522-528 (2020)). Of the NSPs, (1) NSP1 suppresses the antiviral host response, (2) NSP3 is a papain-like protease, (3) NSP5 is a 3CLpro (3C-like protease domain), (4) NSP7 makes a complex with NSP8 to form a primase, (5) NSP9 is responsible for RNA/DNA binding activity, (6) NSP12 is an RNA-dependent RNA polymerase (RdRp), (7) NSP13 is confirmed as a helicase, (8) NSP14 is a 3′-5′ exonuclease (ExoN), (9) NSP15 is a poly(U)-specific endoribonuclease (XendoU). The remaining NSPs are involved in transcription and replication of the viral genome (Chan et al., Emerg. Microbes Infect., 9: 221-236 (2020); and Krichel et al., Biochem. J., 477: 1009-1019 (2019)).

Coronavirus spike proteins are known to elicit potent neutralizing-antibody and T-cell responses. The ability of a virus (e.g., coronavirus OC43, coronavirus 229E, coronavirus NL63, coronavirus HKU1, MERS-CoV, SARS-CoV, or SARS-CoV-2 (COVID-19)) to gain entry into cells and establish infection is mediated by the interactions of its Spike glycoproteins with human cell surface receptors. In the case of coronaviruses, Spike proteins are large type I transmembrane protein trimers that protrude from the surface of coronavirus virions. Each Spike protein comprises a large ectodomain (comprising S1 and S2), a transmembrane anchor, and a short intracellular tail. The S1 subunit of the ectodomain mediates binding of the virion to host cell-surface receptors through its receptor-binding domain (RBD). The S2 subunit fuses with both host and viral membranes, by undergoing structural changes.

SARS-CoV-2 utilizes the Spike glycoprotein to interact with cellular receptor ACE2 (Zhou et al., Nature 579: 270-273, doi:10.1038/s41586-020-2012-7 (2020); Hoffmann et al., Cell, S0092-8674 (0020)30229-30224, doi:10.1016/j.cell.2020.02.052 (2020) doi:10.1016/j.cell.2020.02.052 (2020)). The amino acid sequence of the SARS-CoV-2 spike protein has been deposited with the National Center for Biotechnology Information (NCBI) under Accession No. QHD43416. Binding with ACE2 triggers a cascade of cell membrane fusion events for viral entry. The high-resolution structure of SARS-CoV-2 RBD bound to the N-terminal peptidase domain of ACE2 has recently been determined, and the overall ACE2-binding mechanism is virtually the same between SARS-CoV-2 and SARS-CoV RBDs, indicating convergent ACE2-binding evolution between these two viruses (Gui et al., CellRes 27, 119-129, doi:10.1038/cr.2016.152 (2017); Song et al., PLoS Pathog 14, e1007236-e1007236, doi:10.1371/journal.ppat.1007236 (2018); Yuan et al., Nat Commun 8, 15092-15092, doi:10.1038/ncomms15092 (2017); and Wan et al., J Virol, JVI.00127-00120, doi:10.1128/JVI.00127-20 (2020)).

### Immunogenic Compositions

The disclosure provides immunogenic compositions comprising a nanoemulsion, an agonist of retinoic acid-inducible gene I (RIG-I) and/or an agonist of a toll-like receptor (TLR), and/or a coronavirus vaccine. The nanoemulsion, RIG-I agonist and/or TLR agonist, and coronavirus vaccine may be separately formulated as individual compositions, or may be formulated together in any combination. In some aspects, for example, the coronavirus vaccine, the nanoemulsion, the agonist of RIG-I, and/or the agonist of a TLR are present in the same composition. In other aspects, the coronavirus vaccine is present in a first composition, and the nanoemulsion, agonist of RIG-I, and/or agonist of a TLR are present in a second composition. In yet further aspects, each of the coronavirus vaccine, the nanoemulsion, the agonist of RIG-I, and/or the agonist of a TLR is present in a separate compositions.

The disclosure further provides prophylactic and therapeutic methods comprising administering to a subject in need thereof an immunogenic composition of the disclosure comprising a nanoemulsion, an agonist of retinoic acid-inducible gene I (RIG-I) and/or an agonist of a TLR, and a coronavirus vaccine.

### Coronavirus Vaccines

Any type of vaccine directed against any type of coronavirus may be administered to a subject using an immunogenic composition of the disclosure, such as a human that has been exposed to, or is suspected of exposure to, a coronavirus, and/or a subject at risk for coronavirus infection (e.g., the elderly and/or immunocompromised). In this regard, for example, the coronavirus vaccine may be a protein subunit vaccine (e.g., RBD), an mRNA vaccine, a DNA vaccine, a viral vector vaccine, a live-attenuated virus vaccine, an inactivated virus vaccine, a pseudotyped virus vaccine, etc. While there are no vaccines against SARS-CoV or MERS that have been approved by the U.S. Food and Drug Administration (FDA), a number of such vaccines are in preclinical and clinical trials (Lin et al., Antivir Ther. 2007; 12(7):1107-13; Martin et al., Vaccine. 2008; 26(50):6338-43; Modjarrad et al., Lancet Infect Dis. 2019; 19(9):1013-22; Koch et al., Lancet Infect Dis. 2020; doi.org/10.1016/S1473-3099 (20)30248-6; and Folegatti et al., Lancet Infect Dis. 2020; 20(7):816-26). However, the FDA has authorized three COVID-19 vaccines for emergency use: mRNA-1273 (ModernaTX, Inc.), BNT162b2 (Pfizer, Inc., and BioNTech), and JNJ-78436735 (Janssen Pharmaceuticals, Inc.). The mRNA-1273 and BNT162b2 vaccines are mRNA vaccines, and the JNJ-78436735 vaccine is a viral vector (i.e., adenoviral vector) vaccine. Another viral vector vaccine, Vaxzevria (also referred to as “COVID-19 Vaccine (ChAdOx1-S [recombinant])”) has been authorized for use by the European Medicines Agency (EMA) and other non-U.S. countries. Thus, any of these authorized vaccines may be administered to a subject in accordance with the disclosed method. Several other SARS-CoV-2 vaccines are currently in preclinical and clinical trials (see, e.g., Li et al., Journal of Biomedical Science volume 27, Article number: 104 (2020)), any of which also may be employed in the disclosed compositions and methods.

In some embodiments, the coronavirus vaccine is a protein subunit vaccine, a whole virus vaccine, or a pseudotyped virus vaccine. The term “subunit vaccine,” as used herein, refers to a vaccine composed of protein or glycoprotein components of a pathogen that are capable of inducing a protective immune response, and may be produced by conventional biochemical or recombinant DNA technologies. A “whole virus vaccine” comprises an entire virus that has been killed, attenuated, or weakened so that it cannot cause disease. Whole virus vaccines can elicit strong protective immune responses. A whole virus vaccine may comprise a live cold-adapted virus, which is a virus comprising a temperature sensitive mutation that allows for replication and confers stability in nasal mucosa, but has restricted ability to replicate in the lungs. “Pseudotyping” refers to the process of producing viruses or viral vectors using foreign viral envelope proteins. The resulting virus is referred to as a “pseudotyped virus.” In some cases, the inability to produce viral envelope proteins renders the pseudovirus replication-incompetent, which enables investigation of dangerous viruses in a lower risk setting. Indeed, pseudotyping viral systems have been widely employed to study highly infectious and pathogenic viruses, such as Ebola virus, Middle Eastern Respiratory Syndrome (MERS) virus, or SARS viruses (McWilliams et al., Cell Rep. (2019) 26:1718-26.e4. doi: 10.1016/j.celrep.2019.01.069; Liu et al., Antiviral Res. (2018) 150:30-8. doi: 10.1016/j.antiviral.2017.12.007; and Fukushi et al., SARS- and Other Coronaviruses: Laboratory Protocols. Totowa, NJ: Humana Press (2008). p. 331-8). The two most commonly used pseudotyping systems are retro/lentiviruses and vesicular stomatitis virus (VSV) which lacks the VSV envelope glycoprotein (VSVΔG). The use of replication-restricted pseudoviruses bearing foreign viral coat proteins represents a safe and useful method that has been widely adopted by virologists to study viral entry, detection of neutralizing antibodies in serum samples, and therapeutic development under less stringent biosafety conditions (e.g., biosafety level-2 (BSL-2)). Pseudotyped viruses have been used to produce vaccine candidates against HIV (Racine et al., AIDS Research and Therapy. 14 (1): 55. doi:10.1186/s12981-017-0179-2); Nipah henipavirus (Nie et al., Emerging Microbes & Infections. 8 (1): 272-281; doi:10.1080/22221751.2019.1571871); Rabies lyssavirus (Moeschler et al., Viruses. 8 (9): 254. doi:10.3390/v8090254), SARS-CoV (Kapadia et al., Virology. 376 (1): 165-172. doi:10.1016/j.virol.2008.03.002); Zaire ebolavirus (Salata et al., Viruses. 11 (3): 274. doi:10.3390/v11030274), and SARS-CoV-2 (Johnson et al., Journal of Virology. 94 (21). doi:10.1128/JVI.01062-20; and Condor Capcha et al., Front. Cardiovasc. Med., 15 Jan. (2021)). In some embodiments, a coronavirus vaccine encompassed by the present disclosure may comprise a vesicular stomatitis virus pseudotyped with SARS-CoV-2 spike protein, or a portion thereof. A pseudotyped virus may be further attenuated via the use of misrepresented mammalian codons (referred to as “codon deoptimization”), which also are within the scope of this disclosure. These again haven't been validated by us, but are in our plans for future testing.

In other embodiments, the coronavirus vaccine may be an mRNA vaccine. An “mRNA vaccine”, like the FDA-authorized mRNA-1273 and BNT162b2, is a nucleic acid vaccine based on messenger RNA. The mRNA typically encodes at least one pathogen-specific antigen, and complexed or formulated with carriers (e.g., lipids, polymers) that facilitate cellular uptake of mRNA and protect it from degradation. mRNA vaccine technology is further described in, e.g., Pardi et al., Nature Reviews Drug Discovery volume 17: 261-279 (2018); Schlake et al., RNA Biol. 2012 Nov. 1; 9(11): 1319-1330; and Rahman et al., Vaccines (Basel). 2021 Mar. 11; 9(3):244. doi: 10.3390/vaccines9030244.

The coronavirus vaccine may be a viral vector vaccine. A “viral vector vaccine,” like the FDA-authorized JNJ-78436735 vaccine, consists of a recombinant virus that is often attenuated to reduce its pathogenicity, in which genes encoding viral antigen(s) have been cloned using recombinant DNA techniques. Viral vector vaccines can either be replicating or non-replicating. Replicating vector vaccines infect cells in which the vaccine antigen is produced and are able to replicate and infect new cells that will then also produce the vaccine antigen. Non-replicating vector vaccines initially enter cells and produce the vaccine antigen, but no new virus particles are formed. Because viral vector vaccines result in endogenous antigen production, both humoral and cellular immune responses may be stimulated. Viral vector vaccines may be based on any suitable virus, including, but not limited to, adenovirus, adeno-associated virus (AAV), retrovirus, lentivirus, vaccinia virus, and cytomegalovirus (CMV). Viral vector-based vaccines are described in detail in, e.g., Ura et al., Vaccines (Basel). 2014 September; 2(3): 624-64; Lauer et al., Clin Vaccine Immunol 24:e00298-16; doi.org/10.1128/CVI.00298-16 (2017); and van Riel, D., de Wit, E., Nature Materials, 19: 810-812 (2020).

Whatever type of vaccine is chosen, the vaccine desirably comprises one or more coronavirus antigens, or portions or epitopes thereof. In other embodiments, the vaccine induces a host to produce one or more coronavirus antigens, e.g., by way of comprising one or more nucleic acid sequences encoding one or more coronavirus antigens. In some embodiments, the coronavirus antigen is the SARS-CoV-2 spike protein (“S” protein as provided by, e.g., UniProtKB Accession Number PODTC2) or the spike protein receptor-binding domain (RBD) (see, e.g., Wrapp (2020), Science 367: 1260-63; Walls (2020) Cell 180: 1-12). In some embodiments, the coronavirus antigen is a viral transcription and/or replication protein (e.g., replicase polyprotein 1a (R1a) or replicase polyprotein 1ab (R1ab)). In some embodiments, the coronavirus antigen is a viral budding protein (e.g., protein 3a or envelope small membrane protein (E)). In some embodiments, the coronavirus antigen is a virus morphogenesis protein (e.g., membrane protein (M)). In some embodiments, the coronavirus antigen is non-structural protein 6 (NS6), protein 7a (NS7A), protein 7b (NS7B), non-structural protein 8 (NS8), or protein 9b (NS9B). In some embodiments, the coronavirus antigen is a viral genome packaging protein (e.g., nucleocapsid protein (N or NC)). In some embodiments, the coronavirus antigen is an uncharacterized protein.

In some embodiments, the coronavirus antigen may comprise a protein and/or a nucleic acid, or a portion thereof, from a genetic variant of the SARS-CoV-2 virus, e.g., a SARS-CoV-2 variant of interest, variant of concern, or variant of high consequence. In some embodiments, the variant is B.1.526, B.1.525, P.2, B.1.1.7 (also known as 20I/501Y.V1 and VOC 202012/01), P.1, B.1.351 (also known as 20H/501Y.V2), B.1.427, B.1.429, or B.1.617. SARS-CoV-2 variants are further described in, e.g., Zhou et al., Nature (Feb. 26, 2021); Volz et al., Cell 2021; 184 (64-75); Korber et al., Cell 2021; 182 (812-7); Davies et al., MedRXiv 2021; Horby et al., New & Emerging Threats Advisory Group, Jan. 21, 2021; Emary et al., Lancet (Feb. 4, 2021); Fact Sheet For Health Care Providers Emergency Use Authorization (EUA) Of Regen-Cov (fda.gov); Wang P, Wang M, Yu J, et al. Increased Resistance of SARS-CoV-2 Variant P.1 to Antibody Neutralization. BioRxiv 2021; and Li et al., Innovation (NY). 2021 May 11; 100116. doi: 10.1016/j.xinn.2021.100116).

A coronavirus vaccine may comprise one or more nucleic acid and/or amino acid sequences that is at least about 70% identical (e.g., about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical) to any of the aforementioned coronavirus antigens. The degree of nucleic acid and/or amino acid identity can be determined using any method known in the art, such as the BLAST sequence database.

### Nanoemulsions

Nanoemulsion formulations described herein are simply examples to illustrate the variety of nanoemulsion adjuvants that find use in the present disclosure. The present disclosure contemplates that many variations of these formulations, as well as additional nanoemulsions, may be used in the methods of the present disclosure. Candidate nanoemulsions can be easily tested to determine if they are suitable for use in the compositions described herein.

Nanoemulsion formulations encompassed by the present disclosure generally are non-toxic (e.g., to humans, plants, or animals), non-irritant (e.g., to humans, plants, or animals), and non-corrosive (e.g., to humans, plants, or animals or the environment), and retain stability when mixed with other agents (e.g., a composition comprising an immunogen (e.g., bacteria, fungi, viruses, and spores).

The nanoemulsion can comprise an aqueous phase, at least one oil, at least one surfactant, and at least one solvent. Nanoemulsions of the present disclosure may comprise the following properties and components.

The nanoemulsion of the present disclosure may comprise droplets having an average diameter size of less than about 1000 nm, less than about 950 nm, less than about 900 nm, less than about 850 nm, less than about 800 nm, less than about 750 nm, less than about 700 nm, less than about 650 nm, less than about 600 nm, less than about 550 nm, less than about 500 nm, less than about 450 nm, less than about 400 nm, less than about 350 nm, less than about 300 nm, less than about 250 nm, less than about 200 nm, less than about 150 nm, or any combination thereof. In one embodiment, the droplets have an average diameter size greater than about 125 nm and less than or equal to about 600 nm. In other embodiments, the droplets have an average diameter size greater than about 50 nm or greater than about 70 nm, and less than or equal to about 125 nm.

The aqueous phase of the nanoemulsion can comprise any type of aqueous phase including, but not limited to, water (e.g., H₂O, distilled water, purified water, water for injection, de-ionized water, tap water) and solutions (e.g., phosphate buffered saline (PBS) solution). In certain embodiments, the aqueous phase comprises water at a pH of about 4 to 10, preferably about 6 to 8. The water can be deionized (hereinafter “DiH₂O”). In some embodiments the aqueous phase comprises phosphate buffered saline (PBS). The aqueous phase may further be sterile and pyrogen free.

Organic solvents in the nanoemulsion can include, but are not limited to, C₁-C₁₂ alcohol, diol, triol, dialkyl phosphate, tri-alkyl phosphate, such as tri-n-butyl phosphate, semi-synthetic derivatives thereof, and combinations thereof. In one aspect, the organic solvent is an alcohol chosen from a nonpolar solvent, a polar solvent, a protic solvent, or an aprotic solvent. Suitable organic solvents include, but are not limited to, ethanol, methanol, isopropyl alcohol, glycerol, medium chain triglycerides, diethyl ether, ethyl acetate, acetone, dimethyl sulfoxide (DMSO), acetic acid, n-butanol, butylene glycol, perfumers alcohols, isopropanol, n-propanol, formic acid, propylene glycols, glycerol, sorbitol, industrial methylated spirit, triacetin, hexane, benzene, toluene, diethyl ether, chloroform, 1,4-dixoane, tetrahydrofuran, dichloromethane, acetone, acetonitrile, dimethylformamide, dimethyl sulfoxide, formic acid, semi-synthetic derivatives thereof, and any combination thereof.

The oil in the nanoemulsion can be any cosmetically or pharmaceutically acceptable oil. The oil can be volatile or non-volatile, and may be chosen from animal oil, vegetable oil, natural oil, synthetic oil, hydrocarbon oils, silicone oils, semi-synthetic derivatives thereof, and combinations thereof.

Suitable oils include, but are not limited to, mineral oil, squalene oil, flavor oils, silicon oil, essential oils, water insoluble vitamins, Isopropyl stearate, Butyl stearate, Octyl palmitate, Cetyl palmitate, Tridecyl behenate, Diisopropyl adipate, Dioctyl sebacate, Menthyl anthranhilate, Cetyl octanoate, Octyl salicylate, Isopropyl myristate, neopentyl glycol dicarpate cetols, Ceraphyls®, Decyl oleate, diisopropyl adipate, C₁₂₋₁₅ alkyl lactates, Cetyl lactate, Lauryl lactate, Isostearyl neopentanoate, Myristyl lactate, Isocetyl stearoyl stearate, Octyldodecyl stearoyl stearate, Hydrocarbon oils, Isoparaffin, Fluid paraffins, Isododecane, Petrolatum, Argan oil, Canola oil, Chile oil, Coconut oil, corn oil, Cottonseed oil, Flaxseed oil, Grape seed oil, Mustard oil, Olive oil, Palm oil, Palm kernel oil, Peanut oil, Pine seed oil, Poppy seed oil, Pumpkin seed oil, Rice bran oil, Safflower oil, Tea oil, Truffle oil, Vegetable oil, Apricot (kernel) oil, Jojoba oil (Simmondsia chinensis seed oil), Grapeseed oil, Macadamia oil, Wheat germ oil, Almond oil, Rapeseed oil, Gourd oil, Soybean oil, Sesame oil, Hazelnut oil, Maize oil, Sunflower oil, Hemp oil, Bois oil, Kuki nut oil, Avocado oil, Walnut oil, Fish oil, berry oil, allspice oil, juniper oil, seed oil, almond seed oil, anise seed oil, celery seed oil, cumin seed oil, nutmeg seed oil, leaf oil, basil leaf oil, bay leaf oil, cinnamon leaf oil, common sage leaf oil, eucalyptus leaf oil, lemon grass leaf oil, melaleuca leaf oil, oregano leaf oil, patchouli leaf oil, peppermint leaf oil, pine needle oil, rosemary leaf oil, spearmint leaf oil, tea tree leaf oil, thyme leaf oil, wintergreen leaf oil, flower oil, chamomile oil, clary sage oil, clove oil, geranium flower oil, hyssop flower oil, jasmine flower oil, lavender flower oil, manuka flower oil, Marhoram flower oil, orange flower oil, rose flower oil, ylang-ylang flower oil, Bark oil, cassia Bark oil, cinnamon bark oil, sassafras Bark oil, Wood oil, camphor wood oil, cedar wood oil, rosewood oil, sandalwood oil), rhizome (ginger) wood oil, resin oil, frankincense oil, myrrh oil, peel oil, bergamot peel oil, grapefruit peel oil, lemon peel oil, lime peel oil, orange peel oil, tangerine peel oil, root oil, valerian oil, Oleic acid, Linoleic acid, Oleyl alcohol, Isostearyl alcohol, semi-synthetic derivatives thereof, and any combinations thereof.

The oil may further comprise a silicone component, such as a volatile silicone component, which can be the sole oil in the silicone component or can be combined with other silicone and non-silicone, volatile and non-volatile oils. Suitable silicone components include, but are not limited to, methylphenylpolysiloxane, simethicone, dimethicone, phenyltrimethicone (or an organo-modified version thereof), alkylated derivatives of polymeric silicones, cetyl dimethicone, lauryl trimethicone, hydroxylated derivatives of polymeric silicones (e.g., dimethiconol), volatile silicone oils, cyclic and linear silicones, cyclomethicone, derivatives of cyclomethicone, hexamethylcyclotrisiloxane, octamethylcyclotetrasiloxane, decamethylcyclopentasiloxane, volatile linear dimethylpolysiloxanes, isohexadecane, isoeicosane, isotetracosane, polyisobutene, isooctane, isododecane, semi-synthetic derivatives thereof, and combinations thereof.

The volatile oil can be the organic solvent, or the volatile oil can be present in addition to an organic solvent. Suitable volatile oils include, but are not limited to, a terpene, monoterpene, sesquiterpene, carminative, azulene, menthol, camphor, thujone, thymol, nerol, linalool, limonene, geraniol, perillyl alcohol, nerolidol, farnesol, ylangene, bisabolol, farnesene, ascaridole, chenopodium oil, citronellal, citral, citronellol, chamazulene, yarrow, guaiazulene, chamomile, semi-synthetic derivatives, or combinations thereof. In some embodiments, the volatile oil in the silicone component is different than the oil in the oil phase.

Surface active agents, or surfactants, are amphipathic molecules that consist of a non-polar hydrophobic portion, usually a straight or branched hydrocarbon or fluorocarbon chain containing 8-18 carbon atoms, attached to a polar or ionic hydrophilic portion. The hydrophilic portion can be nonionic, ionic or zwitterionic. The hydrocarbon chain interacts weakly with the water molecules in an aqueous environment, whereas the polar or ionic head group interacts strongly with water molecules via dipole or ion-dipole interactions. Based on the nature of the hydrophilic group, surfactants are classified into anionic, cationic, zwitterionic, nonionic and polymeric surfactants.

The surfactant in the nanoemulsion can be a pharmaceutically acceptable ionic surfactant, a pharmaceutically acceptable nonionic surfactant, a pharmaceutically acceptable cationic surfactant, a pharmaceutically acceptable anionic surfactant, or a pharmaceutically acceptable zwitterionic surfactant. Further, the surfactant can be a pharmaceutically acceptable ionic polymeric surfactant, a pharmaceutically acceptable nonionic polymeric surfactant, a pharmaceutically acceptable cationic polymeric surfactant, a pharmaceutically acceptable anionic polymeric surfactant, or a pharmaceutically acceptable zwitterionic polymeric surfactant. Examples of polymeric surfactants include, but are not limited to, a graft copolymer of a poly(methyl methacrylate) backbone with multiple (at least one) polyethylene oxide (PEO) side chain, polyhydroxystearic acid, an alkoxylated alkyl phenol formaldehyde condensate, a polyalkylene glycol modified polyester with fatty acid hydrophobes, a polyester, semi-synthetic derivatives thereof, or combinations thereof. Exemplary surfactants are described in Applied Surfactants: Principles and Applications (Tharwat F. Tadros, Copyright August 2005 WILEY-VCH Verlag GmbH & Co. KGaA, Weinheim ISBN: 3-527-30629-3).

Suitable surfactants include, but are not limited to, ethoxylated nonylphenol comprising 9 to 10 units of ethyleneglycol, ethoxylated undecanol comprising 8 units of ethyleneglycol, polyoxyethylene (20) sorbitan monolaurate, polyoxyethylene (20) sorbitan monopalmitate, polyoxyethylene (20) sorbitan monostearate, polyoxyethylene (20) sorbitan monooleate, sorbitan monolaurate, sorbitan monopalmitate, sorbitan monostearate, sorbitan monooleate, ethoxylated hydrogenated ricin oils, sodium laurylsulfate, a diblock copolymer of ethyleneoxyde and propyleneoxyde, Ethylene Oxide-Propylene Oxide Block Copolymers, and tetra-functional block copolymers based on ethylene oxide and propylene oxide, Glyceryl monoesters, Glyceryl caprate, Glyceryl caprylate, Glyceryl cocate, Glyceryl erucate, Glyceryl hydroxysterate, Glyceryl isostearate, Glyceryl lanolate, Glyceryl laurate, Glyceryl linolate, Glyceryl myristate, Glyceryl oleate, Glyceryl PABA, Glyceryl palmitate, Glyceryl ricinoleate, Glyceryl stearate, Glyceryl thiglycolate, Glyceryl dilaurate, Glyceryl dioleate, Glyceryl dimyristate, Glyceryl disterate, Glyceryl sesuioleate, Glyceryl stearate lactate, Polyoxyethylene cetyl/stearyl ether, Polyoxyethylene cholesterol ether, Polyoxyethylene laurate or dilaurate, Polyoxyethylene stearate or distearate, polyoxyethylene fatty ethers, Polyoxyethylene lauryl ether, Polyoxyethylene stearyl ether, polyoxyethylene myristyl ether, a steroid, Cholesterol, Betasitosterol, Bisabolol, fatty acid esters of alcohols, isopropyl myristate, Aliphati-isopropyl n-butyrate, Isopropyl n-hexanoate, Isopropyl n-decanoate, Isoproppyl palmitate, Octyldodecyl myristate, alkoxylated alcohols, alkoxylated acids, alkoxylated amides, alkoxylated sugar derivatives, alkoxylated derivatives of natural oils and waxes, polyoxyethylene polyoxypropylene block copolymers, nonoxynol-14, PEG-8 laurate, PEG-6 Cocoamide, PEG-20 methylglucose sesquistearate, PEG40 lanolin, PEG-40 castor oil, PEG-40 hydrogenated castor oil, polyoxyethylene fatty ethers, glyceryl diesters, polyoxyethylene stearyl ether, polyoxyethylene myristyl ether, and polyoxyethylene lauryl ether, glyceryl dilaurate, glyceryl dimystate, glyceryl distearate, semi-synthetic derivatives thereof, or mixtures thereof.

Additional suitable surfactants include, but are not limited to, non-ionic lipids, such as glyceryl laurate, glyceryl myristate, glyceryl dilaurate, glyceryl dimyristate, semi-synthetic derivatives thereof, and mixtures thereof.

In other embodiments, the surfactant is a polyoxyethylene fatty ether having a polyoxyethylene head group ranging from about 2 to about 100 groups, or an alkoxylated alcohol having the structure R₅—(OCH₂ CH₂)_{y}—OH, wherein R5 is a branched or unbranched alkyl group having from about 6 to about 22 carbon atoms and y is between about 4 and about 100, and preferably, between about 10 and about 100. Preferably, the alkoxylated alcohol is the species wherein R5 is a lauryl group and y has an average value of 23. In other embodiments, the surfactant is an alkoxylated alcohol which is an ethoxylated derivative of lanolin alcohol. Preferably, the ethoxylated derivative of lanolin alcohol is laneth-10, which is the polyethylene glycol ether of lanolin alcohol with an average ethoxylation value of 10.

Nonionic surfactants include, but are not limited to, an ethoxylated surfactant, an alcohol ethoxylated, an alkyl phenol ethoxylated, a fatty acid ethoxylated, a monoalkaolamide ethoxylated, a sorbitan ester ethoxylated, a fatty amino ethoxylated, an ethylene oxide-propylene oxide copolymer, Bis(polyethylene glycol bis[imidazoyl carbonyl]), nonoxynol-9, Bis(polyethylene glycol bis[imidazoyl carbonyl]), Brij®35, Brij® 56, Brij® 72, Brij® 76, Brij® 92V, Brij® 97, Brij® 58P, Cremophor® EL, Decaethylene glycol monododecyl ether, N-Decanoyl-N-methylglucamine, n-Decyl alpha-D-glucopyranoside, Decyl beta-D-maltopyranoside, n-Dodecanoyl-N-methylglucamide, n-Dodecyl alpha-D-maltoside, n-Dodecyl beta-D-maltoside, n-Dodecyl beta-D-maltoside, Heptaethylene glycol monodecyl ether, Heptaethylene glycol monododecyl ether, Heptaethylene glycol monotetradecyl ether, n-Hexadecyl beta-D-maltoside, Hexaethylene glycol monododecyl ether, Hexaethylene glycol monohexadecyl ether, Hexaethylene glycol monooctadecyl ether, Hexaethylene glycol monotetradecyl ether, Igepal CA-630, Igepal CA-630, Methyl-6-O—(N-heptylcarbamoyl)-alpha-D-glucopyranoside, Nonaethylene glycol monododecyl ether, N-Nonanoyl-N-methylglucamine, N-Nonanoyl-N-methylglucamine, Octaethylene glycol monodecyl ether, Octaethylene glycol monododecyl ether, Octaethylene glycol monohexadecyl ether, Octaethylene glycol monooctadecyl ether, Octaethylene glycol monotetradecyl ether, Octyl-beta-D-glucopyranoside, Pentaethylene glycol monodecyl ether, Pentaethylene glycol monododecyl ether, Pentaethylene glycol monohexadecyl ether, Pentaethylene glycol monohexyl ether, Pentaethylene glycol monooctadecyl ether, Pentaethylene glycol monooctyl ether, Polyethylene glycol diglycidyl ether, Polyethylene glycol ether W-1, Polyoxyethylene 10 tridecyl ether, Polyoxyethylene 100 stearate, Polyoxyethylene 20 isohexadecyl ether, Polyoxyethylene 20 oleyl ether, Polyoxyethylene 40 stearate, Polyoxyethylene 50 stearate, Polyoxyethylene 8 stearate, Polyoxyethylene bis(imidazolyl carbonyl), Polyoxyethylene 25 propylene glycol stearate, Saponin from Quillaja bark, Span® 20, Span® 40, Span® 60, Span® 65, Span® 80, Span® 85, Tergitol, Type 15-S-12, Tergitol, Type 15-S-30, Tergitol, Type 15-S-5, Tergitol, Type 15-S-7, Tergitol, Type 15-S-9, Tergitol, Type NP-10, Tergitol, Type NP-4, Tergitol, Type NP-40, Tergitol, Type NP-7, Tergitol, Type NP-9, Tergitol, Tergitol, Type TMN-10, Tergitol, Type TMN-6, Tetradecyl-beta-D-maltoside, Tetraethylene glycol monodecyl ether, Tetraethylene glycol monododecyl ether, Tetraethylene glycol monotetradecyl ether, Triethylene glycol monodecyl ether, Triethylene glycol monododecyl ether, Triethylene glycol monohexadecyl ether, Triethylene glycol monooctyl ether, Triethylene glycol monotetradecyl ether, Triton CF-21, Triton CF-32, Triton DF-12, Triton DF-16, Triton GR-5M, Triton QS-15, Triton QS-44, Triton X-100, Triton X-102, Triton X-15, Triton X-151, Triton X-200, Triton X-207, Triton® X-100, Triton® X-114, Triton® X-165, Triton® X-305, Triton® X-405, Triton® X-45, Triton® X-705-70, TWEEN® 20, TWEEN® 21, TWEEN® 40, TWEEN® 60, TWEEN® 61, TWEEN® 65, TWEEN® 80, TWEEN® 81, TWEEN® 85, Tyloxapol, n-Undecyl beta-D-glucopyranoside, semi-synthetic derivatives thereof, or combinations thereof.

In other embodiments, the nonionic surfactant can be a poloxamer. Poloxamers are polymers made of a block of polyoxyethylene, followed by a block of polyoxypropylene, followed by a block of polyoxyethylene. The average number of units of polyoxyethylene and polyoxypropylene varies based on the number associated with the polymer. For example, the smallest polymer, Poloxamer 101, consists of a block with an average of 2 units of polyoxyethylene, a block with an average of 16 units of polyoxypropylene, followed by a block with an average of 2 units of polyoxyethylene. Poloxamers range from colorless liquids and pastes to white solids. In cosmetics and personal care products, Poloxamers are used in the formulation of skin cleansers, bath products, shampoos, hair conditioners, mouthwashes, eye makeup remover and other skin and hair products. Examples of Poloxamers include, but are not limited to, Poloxamer 101, Poloxamer 105, Poloxamer 108, Poloxamer 122, Poloxamer 123, Poloxamer 124, Poloxamer 181, Poloxamer 182, Poloxamer 183, Poloxamer 184, Poloxamer 185, Poloxamer 188, Poloxamer 212, Poloxamer 215, Poloxamer 217, Poloxamer 231, Poloxamer 234, Poloxamer 235, Poloxamer 237, Poloxamer 238, Poloxamer 282, Poloxamer 284, Poloxamer 288, Poloxamer 331, Poloxamer 333, Poloxamer 334, Poloxamer 335, Poloxamer 338, Poloxamer 401, Poloxamer 402, Poloxamer 403, Poloxamer 407, Poloxamer 105 Benzoate, and Poloxamer 182 Dibenzoate.

Suitable cationic surfactants include, but are not limited to, a quarternary ammonium compound, an alkyl trimethyl ammonium chloride compound, a dialkyl dimethyl ammonium chloride compound, a cationic halogen-containing compound, such as cetylpyridinium chloride, Benzalkonium chloride, Benzalkonium chloride, Benzyldimethylhexadecylammonium chloride, Benzyldimethyltetradecylammonium chloride, Benzyldodecyldimethylammonium bromide, Benzyltrimethylammonium tetrachloroiodate, Dimethyldioctadecylammonium bromide, Dodecylethyldimethylammonium bromide, Dodecyltrimethylammonium bromide, Dodecyltrimethylammonium bromide, Ethylhexadecyldimethylammonium bromide, Girard's reagent T, Hexadecyltrimethylammonium bromide, Hexadecyltrimethylammonium bromide, N,N′,N′-Polyoxyethylene(10)-N-tallow-1,3-diaminopropane, Thonzonium bromide, Trimethyl(tetradecyl)ammonium bromide, 1,3,5-Triazine-1,3,5 (2H,4H,6H)-triethanol, 1-Decanaminium, N-decyl-N, N-dimethyl-, chloride, Didecyl dimethyl ammonium chloride, 2-(2-(p-(Diisobutyl)cresosxy)ethoxy)ethyl dimethyl benzyl ammonium chloride, 2-(2-(p-(Diisobutyl)phenoxy)ethoxy)ethyl dimethyl benzyl ammonium chloride, Alkyl 1 or 3 benzyl-1-(2-hydroxethyl)-2-imidazolinium chloride, Alkyl bis(2-hydroxyethyl) benzyl ammonium chloride, Alkyl demethyl benzyl ammonium chloride, Alkyl dimethyl 3,4-dichlorobenzyl ammonium chloride (100% C₁₂), Alkyl dimethyl 3,4-dichlorobenzyl ammonium chloride (50% C₁₄, 40% C₁₂, 10% C₁₆), Alkyl dimethyl 3,4-dichlorobenzyl ammonium chloride (55% C₁₄, 23% C₁₂, 20% C₁₆), Alkyl dimethyl benzyl ammonium chloride, Alkyl dimethyl benzyl ammonium chloride (100% C₁₄), Alkyl dimethyl benzyl ammonium chloride (100% C₁₆), Alkyl dimethyl benzyl ammonium chloride (41% C₁₄, 28% C₁₂), Alkyl dimethyl benzyl ammonium chloride (47% C₁₂, 18% C₁₄), Alkyl dimethyl benzyl ammonium chloride (55% C₁₆, 20% C₁₄), Alkyl dimethyl benzyl ammonium chloride (58% C₁₄, 28% C₁₆), Alkyl dimethyl benzyl ammonium chloride (60% C₁₄, 25% C₁₂), Alkyl dimethyl benzyl ammonium chloride (61% C₁₁, 23% C₁₄), Alkyl dimethyl benzyl ammonium chloride (61% C₁₂, 23% C₁₄), Alkyl dimethyl benzyl ammonium chloride (65% C₁₂, 25% C₁₄), Alkyl dimethyl benzyl ammonium chloride (67% C₁₂, 24% C₁₄), Alkyl dimethyl benzyl ammonium chloride (67% C₁₂, 25% C₁₄), Alkyl dimethyl benzyl ammonium chloride (90% C₁₄, 5% C₁₂), Alkyl dimethyl benzyl ammonium chloride (93% C₁₄, 4% C₁₂), Alkyl dimethyl benzyl ammonium chloride (95% C₁₆, 5% C₁₈), Alkyl dimethyl benzyl ammonium chloride, Alkyl didecyl dimethyl ammonium chloride, Alkyl dimethyl benzyl ammonium chloride, Alkyl dimethyl benzyl ammonium chloride (C₁₂₋₁₆), Alkyl dimethyl benzyl ammonium chloride (C₁₂₋₁₈), Alkyl dimethyl benzyl ammonium chloride, dialkyl dimethyl benzyl ammonium chloride, Alkyl dimethyl dimethybenzyl ammonium chloride, Alkyl dimethyl ethyl ammonium bromide (90% C₁₄, 5% C₁₆, 5% C₁₂), Alkyl dimethyl ethyl ammonium bromide (mixed alkyl and alkenyl groups as in the fatty acids of soybean oil), Alkyl dimethyl ethylbenzyl ammonium chloride, Alkyl dimethyl ethylbenzyl ammonium chloride (60% C₁₄), Alkyl dimethyl isopropylbenzyl ammonium chloride (50% C₁₂, 30% C₁₄, 17% C₁₆, 3% Cis), Alkyl trimethyl ammonium chloride (58% Cis, 40% C₁₆, 1% C₁₄, 1% C₁₂), Alkyl trimethyl ammonium chloride (90% Cis, 10% C₁₆), Alkyldimethyl(ethylbenzyl) ammonium chloride (C₁₂₋₁₈), Di-(C₈₋₁₀)-alkyl dimethyl ammonium chlorides, Dialkyl dimethyl ammonium chloride, Dialkyl methyl benzyl ammonium chloride, Didecyl dimethyl ammonium chloride, Diisodecyl dimethyl ammonium chloride, Dioctyl dimethyl ammonium chloride, Dodecyl bis (2-hydroxyethyl) octyl hydrogen ammonium chloride, Dodecyl dimethyl benzyl ammonium chloride, Dodecylcarbamoyl methyl dinethyl benzyl ammonium chloride, Heptadecyl hydroxyethylimidazolinium chloride, Hexahydro-1,3,5-tris(2-hydroxyethyl)-s-triazine, Hexahydro-1,3,5-tris(2-hydroxyethyl)-s-triazine, Myristalkonium chloride (and) Quat RNIUM 14, N,N-Dimethyl-2-hydroxypropylammonium chloride polymer, n-Tetradecyl dimethyl benzyl ammonium chloride monohydrate, Octyl decyl dimethyl ammonium chloride, Octyl dodecyl dimethyl ammonium chloride, Octyphenoxyethoxyethyl dimethyl benzyl ammonium chloride, Oxydiethylenebis(alkyl dimethyl ammonium chloride), Quaternary ammonium compounds, dicoco alkyldimethyl, chloride, Trimethoxysily propyl dimethyl octadecyl ammonium chloride, Trimethoxysilyl quats, Trimethyl dodecylbenzyl ammonium chloride, semi-synthetic derivatives thereof, and combinations thereof.

Exemplary cationic halogen-containing compounds include, but are not limited to, cetylpyridinium halides, cetyltrimethylammonium halides, cetyldimethylethylammonium halides, cetyldimethylbenzylammonium halides, cetyltributylphosphonium halides, dodecyltrimethylammonium halides, or tetradecyltrimethylammonium halides. In some particular embodiments, suitable cationic halogen containing compounds comprise, but are not limited to, cetylpyridinium chloride (CPC), cetyltrimethylammonium chloride, cetylbenzyldimethylammonium chloride, cetylpyridinium bromide (CPB), cetyltrimethylammonium bromide (CTAB), cetyidimethylethylammonium bromide, cetyltributylphosphonium bromide, dodecyltrimethylammonium bromide, and tetrad ecyltrimethylammonium bromide. In particularly preferred embodiments, the cationic halogen containing compound is CPC, although the compositions of the present disclosed are not limited to formulation with an particular cationic containing compound.

Suitable anionic surfactants include, but are not limited to, a carboxylate, a sulphate, a sulphonate, a phosphate, chenodeoxycholic acid, chenodeoxycholic acid sodium salt, cholic acid, ox or sheep bile, Dehydrocholic acid, Deoxycholic acid, Deoxycholic acid, Deoxycholic acid methyl ester, Digitonin, Digitoxigenin, N,N-Dimethyldodecylamine N-oxide, Docusate sodium salt, Glycochenodeoxycholic acid sodium salt, Glycocholic acid hydrate, synthetic, Glycocholic acid sodium salt hydrate, synthetic, Glycodeoxycholic acid monohydrate, Glycodeoxycholic acid sodium salt, Glycodeoxycholic acid sodium salt, Glycolithocholic acid 3-sulfate disodium salt, Glycolithocholic acid ethyl ester, N-Lauroylsarcosine sodium salt, N-Lauroylsarcosine solution, N-Lauroylsarcosine solution, Lithium dodecyl sulfate, Lithium dodecyl sulfate, Lithium dodecyl sulfate, Lugol solution, Niaproof 4, Type 4, 1-Octanesulfonic acid sodium salt, Sodium 1-butanesulfonate, Sodium 1-decanesulfonate, Sodium 1-decanesulfonate, Sodium 1-dodecanesulfonate, Sodium 1-heptanesulfonate anhydrous, Sodium 1-heptanesulfonate anhydrous, Sodium 1-nonanesulfonate, Sodium 1-propanesulfonate monohydrate, Sodium 2-bromoethanesulfonate, Sodium cholate hydrate, Sodium choleate, Sodium deoxycholate, Sodium deoxycholate monohydrate, Sodium dodecyl sulfate, Sodium hexanesulfonate anhydrous, Sodium octyl sulfate, Sodium pentanesulfonate anhydrous, Sodium taurocholate, Taurochenodeoxycholic acid sodium salt, Taurodeoxycholic acid sodium salt monohydrate, Taurohyodeoxycholic acid sodium salt hydrate, Taurolithocholic acid 3-sulfate disodium salt, Tauroursodeoxycholic acid sodium salt, Trizma® dodecyl sulfate, TWEEN® 80, Ursodeoxycholic acid, semi-synthetic derivatives thereof, and combinations thereof.

Suitable zwitterionic surfactants include, but are not limited to, an N-alkyl betaine, lauryl amindo propyl dimethyl betaine, an alkyl dimethyl glycinate, an N-alkyl amino propionate, CHAPS, minimum 98% (TLC), CHAPS, SigmaUltra, minimum 98% (TLC), CHAPS, for electrophoresis, minimum 98% (TLC), CHAPSO, minimum 98%, CHAPSO, SigmaUltra, CHAPSO, for electrophoresis, 3-(Decyldimethylammonio)propanesulfonate inner salt, 3-Dodecyldimethylammonio)propanesulfonate inner salt, SigmaUltra, 3-(Dodecyldimethylammonio)propanesulfonate inner salt, 3-(N,N-Dimethylmyristylammonio)propanesulfonate, 3-(N,N-Dimethyloctadecylammonio)propanesulfonate, 3-(N,N-Dimethyloctylammonio)propanesulfonate inner salt, 3-(N,N-Dimethylpalmitylammonio)propanesulfonate, semi-synthetic derivatives thereof, and combinations thereof.

In some embodiments, the nanoemulsion comprises a cationic surfactant, which can be cetylpyridinium chloride. In other embodiments, the nanoemulsion comprises a cationic surfactant, and the concentration of the cationic surfactant is less than about 5.0% and greater than about 0.001%. In yet another embodiment, the nanoemulsion comprises a cationic surfactant, and the concentration of the cationic surfactant is selected from the group consisting of less than about 5%, less than about 4.5%, less than about 4.0%, less than about 3.5%, less than about 3.0%, less than about 2.5%, less than about 2.0%, less than about 1.5%, less than about 1.0%, less than about 0.90%, less than about 0.80%, less than about 0.70%, less than about 0.60%, less than about 0.50%, less than about 0.40%, less than about 0.30%, less than about 0.20%, or less than about 0.10%. Further, the concentration of the cationic agent in the nanoemulsion is greater than about 0.002%, greater than about 0.003%, greater than about 0.004%, greater than about 0.005%, greater than about 0.006%, greater than about 0.007%, greater than about 0.008%, greater than about 0.009%, greater than about 0.010%, or greater than about 0.001%. In one embodiment, the concentration of the cationic agent in the nanoemulsion is less than about 5.0% and greater than about 0.001%.

In another embodiment, the nanoemulsion comprises at least one cationic surfactant and at least one non-cationic surfactant. The non-cationic surfactant is a nonionic surfactant, such as a polysorbate (Tween), such as polysorbate 80 or polysorbate 20. In one embodiment, the non-ionic surfactant is present in a concentration of about 0.01% to about 5.0%, or the non-ionic surfactant is present in a concentration of about 0.1% to about 3%. In yet another embodiment, the nanoemulsion comprises a cationic surfactant present in a concentration of about 0.01% to about 2%, in combination with a nonionic surfactant.

The nanoemulsion may further comprise additional components, including, for example, one or more solvents, such as an organic phosphate-based solvent, bulking agents, coloring agents, pharmaceutically acceptable excipients, a preservative, pH adjuster, buffer, chelating agent, etc. The additional components can be admixed into a previously emulsified nanoemulsion composition, or the additional components can be added to the original mixture to be emulsified. In certain of these embodiments, one or more additional components are admixed into an existing nanoemulsion composition immediately prior to its use.

Suitable preservatives in the nanoemulsion include, but are not limited to, cetylpyridinium chloride, benzalkonium chloride, benzyl alcohol, chlorhexidine, imidazolidinyl urea, phenol, potassium sorbate, benzoic acid, bronopol, chlorocresol, paraben esters, phenoxyethanol, sorbic acid, alpha-tocophernol, ascorbic acid, ascorbyl palmitate, butylated hydroxyanisole, butylated hydroxytoluene, sodium ascorbate, sodium metabisulphite, citric acid, edetic acid, semi-synthetic derivatives thereof, and combinations thereof. Other suitable preservatives include, but are not limited to, benzyl alcohol, chlorhexidine (bis (p-chlorophenyldiguanido) hexane), chlorphenesin (3-(-4-chloropheoxy)-propane-1,2-diol), Kathon CG (methyl and methylchloroisothiazolinone), parabens (methyl, ethyl, propyl, butyl hydrobenzoates), phenoxyethanol (2-phenoxyethanol), sorbic acid (potassium sorbate, sorbic acid), Phenonip (phenoxyethanol, methyl, ethyl, butyl, propyl parabens), Phenoroc (phenoxyethanol 0.73%, methyl paraben 0.2%, propyl paraben 0.07%), Liquipar Oil (isopropyl, isobutyl, butylparabens), Liquipar PE (70% phenoxyethanol, 30% liquipar oil), Nipaguard MPA (benzyl alcohol (70%), methyl & propyl parabens), Nipaguard MPS (propylene glycol, methyl & propyl parabens), Nipasept (methyl, ethyl and propyl parabens), Nipastat (methyl, butyl, ethyl and propyel parabens), Elestab 388 (phenoxyethanol in propylene glycol plus chlorphenesin and methylparaben), and Killitol (7.5% chlorphenesin and 7.5% methyl parabens).

The nanoemulsion may further comprise at least one pH adjuster. Suitable pH adjusters that may be used in the nanoemulsion include, but are not limited to, diethyanolamine, lactic acid, monoethanolamine, triethylanolamine, sodium hydroxide, sodium phosphate, semi-synthetic derivatives thereof, and combinations thereof.

In some embodiments, the nanoemulsion can comprise a chelating agent. The chelating agent may be present in an amount of about 0.0005% to about 1%. Examples of suitable chelating agents include, but are not limited to, ethylenediamine, ethylenediaminetetraacetic acid (EDTA), phytic acid, polyphosphoric acid, citric acid, gluconic acid, acetic acid, lactic acid, and dimercaprol, and a preferred chelating agent is ethylenediaminetetraacetic acid.

The nanoemulsion may further comprise a buffering agent, such as a pharmaceutically acceptable buffering agent. Examples of suitable buffering agents include, but are not limited to, 2-Amino-2-methyl-1,3-propanediol, ≥99.5% (NT), 2-Amino-2-methyl-1-propanol, ≥99.0% (GC), L-(+)-Tartaric acid, ≥99.5% (T), ACES, ≥99.5% (T), ADA, ≥99.0% (T), Acetic acid, ≥99.5% (GC/T), Acetic acid, for luminescence, ≥99.5% (GC/T), Ammonium acetate solution, for molecular biology, ˜5 M in H₂O, Ammonium acetate, for luminescence, ≥99.0% (calc. on dry substance, T), Ammonium bicarbonate, ≥99.5% (T), Ammonium citrate dibasic, ≥99.0% (T), Ammonium formate solution, 10 M in H₂O, Ammonium formate, ≥99.0% (calc. based on dry substance, NT), Ammonium oxalate monohydrate, ≥99.5% (RT), Ammonium phosphate dibasic solution, 2.5 M in H₂O, Ammonium phosphate dibasic, ≥99.0% (T), Ammonium phosphate monobasic solution, 2.5 M in H₂O, Ammonium phosphate monobasic, ≥99.5% (T), Ammonium sodium phosphate dibasic tetrahydrate, ≥99.5% (NT), Ammonium sulfate solution, for molecular biology, 3.2 M in H₂O, Ammonium tartrate dibasic solution, 2 M in H₂O (colorless solution at 20° C.), Ammonium tartrate dibasic, ≥99.5% (T), BES buffered saline, for molecular biology, 2× concentrate, BES, ≥99.5% (T), BES, for molecular biology, ≥99.5% (T), BICINE buffer Solution, for molecular biology, 1 M in H₂O, BICINE, ≥99.5% (T), BIS-TRIS, ≥99.0% (NT), Bicarbonate buffer solution, ≥0.1 M Na₂CO₃, ≥0.2 M NaHCO₃, Boric acid, ≥99.5% (T), Boric acid, for molecular biology, ≥99.5% (T), CAPS, ≥99.0% (TLC), CHES, ≥99.5% (T), Calcium acetate hydrate, ≥99.0% (calc. on dried material, KT), Calcium carbonate, precipitated, ≥99.0% (KT), Calcium citrate tribasic tetrahydrate, ≥98.0% (calc. on dry substance, KT), Citrate Concentrated Solution, for molecular biology, 1 M in H₂O, Citric acid, anhydrous, 99.5% (T), Citric acid, for luminescence, anhydrous, ≥99.5% (T), Diethanolamine, ≥99.5% (GC), EPPS, ≥99.0% (T), Ethylenediaminetetraacetic acid disodium salt dihydrate, for molecular biology, 99.0% (T), Formic acid solution, 1.0 M in H₂O, Gly-Gly-Gly, ≥99.0% (NT), Gly-Gly, ≥99.5% (NT), Glycine, ≥99.0% (NT), Glycine, for luminescence, ≥99.0% (NT), Glycine, for molecular biology, ≥99.0% (NT), HEPES buffered saline, for molecular biology, 2× concentrate, HEPES, ≥99.5% (T), HEPES, for molecular biology, ≥99.5% (T), Imidazole buffer Solution, 1 M in H₂O, Imidazole, ≥99.5% (GC), Imidazole, for luminescence, ≥99.5% (GC), Imidazole, for molecular biology, ≥99.5% (GC), Lipoprotein Refolding Buffer, Lithium acetate dihydrate, ≥99.0% (NT), Lithium citrate tribasic tetrahydrate, ≥99.5% (NT), MES hydrate, ≥99.5% (T), MES monohydrate, for luminescence, ≥99.5% (T), MES solution, for molecular biology, 0.5 M in H₂O, MOPS, ≥99.5% (T), MOPS, for luminescence, ≥99.5% (T), MOPS, for molecular biology, ≥99.5% (T), Magnesium acetate solution, for molecular biology, ˜1 M in H₂O, Magnesium acetate tetrahydrate, ≥99.0% (KT), Magnesium citrate tribasic nonahydrate, ≥98.0% (calc. based on dry substance, KT), Magnesium formate solution, 0.5 M in H₂O, Magnesium phosphate dibasic trihydrate, ≥98.0% (KT), Neutralization solution for the in-situ hybridization for in-situ hybridization, for molecular biology, Oxalic acid dihydrate, ≥99.5% (RT), PIPES, ≥99.5% (T), PIPES, for molecular biology, ≥99.5% (T), Phosphate buffered saline, solution (autoclaved), Phosphate buffered saline, washing buffer for peroxidase conjugates in Western Blotting, 10× concentrate, Piperazine, anhydrous, ≥99.0% (T), Potassium D-tartrate monobasic, ≥99.0% (T), Potassium acetate solution, for molecular biology, Potassium acetate solution, for molecular biology, 5M in H₂O, Potassium acetate solution, for molecular biology, ˜1 M in H₂O, Potassium acetate, ≥99.0% (NT), Potassium acetate, for luminescence, ≥99.0% (NT), Potassium acetate, for molecular biology, ≥99.0% (NT), Potassium bicarbonate, ≥99.5% (T), Potassium carbonate, anhydrous, ≥99.0% (T), Potassium chloride, ≥99.5% (AT), Potassium citrate monobasic, ≥99.0% (dried material, NT), Potassium citrate tribasic solution, 1 M in H₂O, Potassium formate solution, 14 M in H₂O, Potassium formate, ≥99.5% (NT), Potassium oxalate monohydrate, ≥99.0% (RT), Potassium phosphate dibasic, anhydrous, ≥99.0% (T), Potassium phosphate dibasic, for luminescence, anhydrous, ≥99.0% (T), Potassium phosphate dibasic, for molecular biology, anhydrous, ≥99.0% (T), Potassium phosphate monobasic, anhydrous, ≥99.5% (T), Potassium phosphate monobasic, for molecular biology, anhydrous, ≥99.5% (T), Potassium phosphate tribasic monohydrate, ≥95% (T), Potassium phthalate monobasic, 99.5% (T), Potassium sodium tartrate solution, 1.5 M in H₂O, Potassium sodium tartrate tetrahydrate, ≥99.5% (NT), Potassium tetraborate tetrahydrate, ≥99.0% (T), Potassium tetraoxalate dihydrate, ≥99.5% (RT), Propionic acid solution, 1.0 M in H₂O, STE buffer solution, for molecular biology, pH 7.8, STET buffer solution, for molecular biology, pH 8.0, Sodium 5,5-diethylbarbiturate, ≥99.5% (NT), Sodium acetate solution, for molecular biology, ˜3 M in H₂O, Sodium acetate trihydrate, ≥99.5% (NT), Sodium acetate, anhydrous, ≥99.0% (NT), Sodium acetate, for luminescence, anhydrous, ≥99.0% (NT), Sodium acetate, for molecular biology, anhydrous, ≥99.0% (NT), Sodium bicarbonate, ≥99.5% (T), Sodium bitartrate monohydrate, ≥99.0% (T), Sodium carbonate decahydrate, ≥99.5% (T), Sodium carbonate, anhydrous, ≥99.5% (calc. on dry substance, T), Sodium citrate monobasic, anhydrous, ≥99.5% (T), Sodium citrate tribasic dihydrate, ≥99.0% (NT), Sodium citrate tribasic dihydrate, for luminescence, ≥99.0% (NT), Sodium citrate tribasic dihydrate, for molecular biology, ≥99.5% (NT), Sodium formate solution, 8 M in H₂O, Sodium oxalate, ≥99.5% (RT), Sodium phosphate dibasic dihydrate, ≥99.0% (T), Sodium phosphate dibasic dihydrate, for luminescence, ≥99.0% (T), Sodium phosphate dibasic dihydrate, for molecular biology, ≥99.0% (T), Sodium phosphate dibasic dodecahydrate, ≥99.0% (T), Sodium phosphate dibasic solution, 0.5 M in H₂O, Sodium phosphate dibasic, anhydrous, ≥99.5% (T), Sodium phosphate dibasic, for molecular biology, ≥99.5% (T), Sodium phosphate monobasic dihydrate, ≥99.0% (T), Sodium phosphate monobasic dihydrate, for molecular biology, ≥99.0% (T), Sodium phosphate monobasic monohydrate, for molecular biology, ≥99.5% (T), Sodium phosphate monobasic solution, 5 M in H₂O, Sodium pyrophosphate dibasic, ≥99.0% (T), Sodium pyrophosphate tetrabasic decahydrate, ≥99.5% (T), Sodium tartrate dibasic dihydrate, ≥99.0% (NT), Sodium tartrate dibasic solution, 1.5 M in H₂O (colorless solution at 20° C.), Sodium tetraborate decahydrate, ≥99.5% (T), TAPS, ≥99.5% (T), TES, ≥99.5% (calc. based on dry substance, T), TM buffer solution, for molecular biology, pH 7.4, TNT buffer solution, for molecular biology, pH 8.0, TRIS Glycine buffer solution, 10× concentrate, TRIS acetate—EDTA buffer solution, for molecular biology, TRIS buffered saline, 10× concentrate, TRIS glycine SDS buffer solution, for electrophoresis, 10× concentrate, TRIS phosphate-EDTA buffer solution, for molecular biology, concentrate, 10× concentrate, Tricine, ≥99.5% (NT), Triethanolamine, ≥99.5% (GC), Triethylamine, ≥99.5% (GC), Triethylammonium acetate buffer, volatile buffer, ˜1.0 M in H₂O, Triethylammonium phosphate solution, volatile buffer, ˜1.0 M in H₂O, Trimethylammonium acetate solution, volatile buffer, ˜1.0 M in H₂O, Trimethylammonium phosphate solution, volatile buffer, ˜1 M in H₂O, Tris-EDTA buffer solution, for molecular biology, concentrate, 100× concentrate, Tris-EDTA buffer solution, for molecular biology, pH 7.4, Tris-EDTA buffer solution, for molecular biology, pH 8.0, Trizma® acetate, ≥99.0% (NT), Trizma® base, ≥99.8% (T), Trizma® base, ≥99.8% (T), Trizma® base, for luminescence, ≥99.8% (T), Trizma® base, for molecular biology, ≥99.8% (T), Trizma® carbonate, ≥98.5% (T), Trizma® hydrochloride buffer solution, for molecular biology, pH 7.2, Trizma® hydrochloride buffer solution, for molecular biology, pH 7.4, Trizma® hydrochloride buffer solution, for molecular biology, pH 7.6, Trizma® hydrochloride buffer solutio, for molecular biology, pH 8.0, Trizma® hydrochloride, ≥99.0% (AT), Trizma® hydrochloride, for luminescence, ≥99.0% (AT), Trizma® hydrochloride, for molecular biology, ≥99.0% (AT), and Trizma® maleate, ≥99.5% (NT).

In some embodiments, the nanoemulsion can comprise one or more emulsifying agents to aid in the formation of emulsions. Emulsifying agents include compounds that aggregate at the oil/water interface to form a kind of continuous membrane that prevents direct contact between two adjacent droplets. The nanoemulsion may readily be diluted with water or another aqueous phase to a desired concentration without impairing its desired properties.

### RIG-I and TLR Agonists

Recombinant protein-based SARS-CoV-2 vaccines have been observed to be poorly immunogenic. Some results with adjuvanted recombinant SARS-CoV-2 protein vaccines in clinical trials have been reported. Adjuvants also have been reported to broaden vaccine responses, such as against antigenically drifted influenza viruses. In addition, the use of adjuvants can allow for antigen dose sparing, which is an important feature during a pandemic caused by a newly emerging pathogen like SARS-CoV-2 when vaccines are unavailable or scarce. Adjuvants function through the induction of innate immune pathways, thereby providing an optimal cytokine and chemokine environment that promotes the induction of quantitatively and qualitatively improved immune responses. For viruses that induce long-lasting immunity, natural viral infection stimulates strong innate immune responses through the activation of three main pathways involving Toll-, RIG-I-, and NOD-like receptors (TLRs, RLRs, NLRs). However, SARS-CoV-2 infection results in a large variability in magnitude of immune responses in recovered patients, with most patients having relatively stable antibody titers for at least 8 months, but others experiencing rapid waning of antibodies after convalescence. SARS-CoV-2 and SARS-CoV infections induce a muted innate response, with weaker induction of key cytokines and poor activation of type-I interferons (IFN-Is) pathways. IFN-Is are the master activators of the antiviral defense program, and have an essential role in priming adaptive T cell responses and in shaping effector and memory T cell pools. SARS-CoV-2 and SARS-CoV both employ host immunity evasion tactics of inhibiting IFN-I producing pathways at multiple points, including direct inhibition of RIG-I/MAVS as well as inhibiting downstream effector molecules. Furthermore, these viruses have strategies to avoid recognition of their RNA by RLRs. This weak innate response likely contributes to the large variability in magnitude of immune responses in infected patients and duration of protection.

TLR signaling also drives T cell responses and promotes affinity maturation of antiviral antibodies. Multivalent stimulation of TLRs through combined agonists has been shown to enhance antibody responses in a SARS-CoV vaccine, and skewed responses towards a more TH1 response. Thus, in some aspects, an immunogenic composition described herein comprises an agonist of retinoic acid-inducible gene I (RIG-I) and/or an agonist of a toll-like receptor. RIG-I is an intracellular molecule that responds to viral nucleic acids and activates downstream signaling, resulting in the induction of members of the type I interferon (IFN) family. RIG-I is a member of the pattern-recognition receptors (PRRs) family of proteins, which includes toll-like receptor (TLR) proteins. RIG-I belongs to the cytosolic DExD/H box RNA helicases and is one of three members of the so-called family of RIG-I-like helicases (others include MDA5 and LGP2). RIG-I is closely related to the Dicer family of helicases of the RNAi pathway. RIG-I contains a RNA helicase domain and a two N-terminal CARD domains which relay the signal to the downstream signaling adaptor mitochondrial antiviral-signaling protein (MAVS). RIG-I signaling via MAVS not only leads to the induction of type I IFN responses via TBK1 and IRF7/8, but it also activates caspase-8-dependent apoptosis, preferentially in tumor. Furthermore, RIG-I has been shown to mediate MAVS-independent inflammasome activation, specifically in the context of viral infection. RIG-I structure and function is further described in, e.g., Matsumiya T, Stafforini DM., Crit Rev Immunol. 2010; 30(6):489-513. doi:10.1615/critrevimmunol.v30.i6.10; and Rehwinkel, J., Gack, M. U., Nat Rev Immunol 20, 537-551 (2020); doi.org/10.1038/s41577-020-0288-3).

Investigations into ligand recognition by the RIG-I protein have elucidated a number of ligands that serve as agonists and antagonists for RIG-I (see, e.g., Ranjith-Kumar et al., J Biol Chem. 2009 Jan. 9; 284(2): 1155-1165). The term “agonist,” as used herein, refers to a molecule, substance, or compound that binds to a receptor and activates the receptor to produce a biological response. In contrast, the term “antagonist” refers to a molecule, substance, or compound that inhibits or blocks the activity of a receptor to which it binds. Any suitable RIG-I agonist may be included in the nanoemulsion-containing composition. In some embodiments, the RIG-I agonist is a substance or compound that mimics the pathogen-associated molecular pattern (PAMP) induced by a natural viral infection. In some embodiments, the RIG-I agonist is an RNA agonist. Exemplary RIG-I RNA agonists include single-stranded and double-stranded RNAs, such as those described in Ranjith-Kumar et al., supra. In another embodiment, the RNA agonist may be a defective interfering (DI) RNA of a Sendai virus (IVT DI) or an influenza virus 5′ triphosphate hairpin RNA (3phpRNA (InvivoGen, San Diego, CA). IVT DI is an in vitro transcribed RNA consisting of the full-length (546 nt) copy-back defective interfering RNA of Sendai virus strain Cantell (see, e.g., Martinez-Gil et al., J Virol 2013, 87 (3), 1290-300; and Patel et al., EMBO reports 2013, 14 (9), 780-7). The hairpin structure of IVT DI, along with its dsRNA panhandle and 5′ triphosphate, make it a potent and selective RIG-I agonist, and thus, a strong inducer of IFN-Is and interferon-stimulated genes (ISGs). In other embodiments, the RIG-I agonist may be a small molecule. Any suitable small molecule RIG-I agonist may be used, several of which are known in the art (see, e.g., Loo et al., Cytokine, 70, Issue 1, November 2014, Page 56; and Hemann et al., J Immunol May 1, 2016, 196 (1 Supplement) 76.1).

In other embodiments, the immunogenic composition may comprise an agonist of a toll-like receptor (TLR). Any suitable agonist of any suitable toll-like receptor (such as those described herein) may be included in the nanoemulsion-containing composition. For example, a polyriboinosinic polyribocytidylic (pIC) adjuvant activates TLR3 and the RLR MDA5, the synthetic oligodeoxynucleotide CpG is a TLR9 agonist, and the monophosphoryl lipid A stimulates TLR4 signaling (Evans et al., Expert Rev. Vaccines 2:219-229 (2003)). In some embodiments, the TLR agonist is an agonist of TLR3. For example, the TLR3 agonist may be a synthetic double-stranded RNA polyriboinosinic polyribocytidylic acid (also referred to as “pIC,” “poly(I:C),” “poly I:C,” and “p(I:C)”.) pIC is a double-stranded RNA that elicits an immune response by activating toll-like receptor 3 (TLR3), and has long been known as a potent inducer of type I IFN for decades (Field et al., PNAS, 58(5): 2102-2108 (1967)). pIC also has been shown to engage the cytosolic helicase MDA-5. Although originally deemed too toxic for human use, recently the potent adjuvant activity of poly I:C has been newly appreciated in vaccine formulations targeted to dendritic cells (DCs) (Trumpfheller et al., Proc Natl Acad Sci USA, 105(7):2574-9 (2008). doi: 10.1073/pnas.07119761052008). Small molecule TLR agonists also may be employed in the disclosed compositions and methods, several of which are known in the art (see, e.g., Zhang et al., J Med Chem. 2017 Jun. 22; 60(12):5029-5044. doi: 10.1021/acs.jmedchem.7b00419; Wang et al., Chem. Soc. Rev., 2013, 42, 4859-4866; and Shukla et al., ACS Med. Chem. Lett. 2018, 9, 12, 1156-1159).

### Compositions and Methods for Inducing an Immune Response

As described herein, the disclosure provides an immunogenic composition comprising: (a) a nanoemulsion; (b) an agonist of retinoic acid-inducible gene I (RIG-I) and/or an agonist of a toll-like receptor; and (c) a coronavirus vaccine. The disclosure, however, is not limited to an immunogenic composition comprising all four of the nanoemulsion, the RIG-I agonist and/or the TLR agonist, and the coronavirus vaccine. Indeed, the disclosure encompasses an immunogenic composition comprising each of the nanoemulsion, the RIG-I agonist, the TLR agonist, and the coronavirus vaccine individually (with an appropriate pharmaceutically acceptable carrier), or in any combination. For example, the coronavirus vaccine may present in a first composition, while the nanoemulsion, agonist of RIG-I, and/or agonist of a TLR may be present in a second composition. Alternatively, the coronavirus vaccine and the nanoemulsion may be present in a first composition, while the RIG-I agonist and/or TLR agonist (e.g., TLR3 agonist) may be present in a second composition.

A composition of the present disclosure desirably is a pharmaceutically acceptable (e.g., physiologically acceptable) composition, which comprises a carrier, preferably a pharmaceutically acceptable (e.g., physiologically acceptable) carrier, the nanoemulsion, the RIG-I agonist and/or the TLR agonist, and/or the coronavirus vaccine. Compositions of the present disclosure may be formulated into pharmaceutical compositions that are administered in a therapeutically effective amount to a subject and may further comprise suitable, pharmaceutically-acceptable excipients, additives, or preservatives. Suitable excipients, additives, and preservatives are well known in the art.

The compositions described herein desirably comprise therapeutically effective amounts of the coronavirus vaccine, the nanoemulsion, the RIG-I agonist, and/or the TLR agonist. A “therapeutically effective amount” refers to an amount effective, at dosages and for periods of time necessary, to achieve a desired therapeutic result. Alternatively, the pharmacologic and/or physiologic effect may be prophylactic, i.e., the effect completely or partially prevents a disease or symptom thereof. In this respect, the disclosed compositions comprise “prophylactically effective amounts” of the coronavirus vaccine, the nanoemulsion, the RIG-I agonist, and/or the TLR agonist. A “prophylactically effective amount” refers to an amount effective, at dosages and for periods of time necessary, to achieve a desired prophylactic result (e.g., prevention of subsequent infection and/or disease onset).

Exemplary dosage forms for pharmaceutical administration are described herein, and include, but are not limited to liquids, ointments, creams, emulsions, lotions, gels, bioadhesive gels, sprays, aerosols, pastes, foams, sunscreens, capsules, microcapsules, suspensions, pessary, powder, semi-solid dosage forms, etc. The compositions can be generated in accordance with conventional techniques described in, e.g., Remington: The Science and Practice of Pharmacy, 23rd Edition, Academic Press (2020).

The disclosed compositions can be provided in many different types of containers and delivery systems. For example, in some embodiments, the composition can be presented in unit-dose or multi-dose sealed containers, such as ampules and vials, and can be stored in a freeze-dried (lyophilized) condition requiring only the addition of the sterile liquid carrier, for example, water, immediately prior to use. In some embodiments, the compositions are provided in a suspension or liquid form. Such compositions can be delivered in any suitable container including spray bottles and any suitable pressurized spray device. Such spray bottles may be suitable for delivering the compositions intranasally or via inhalation. These containers can further be packaged with instructions for use to form kits (described below).

The disclosure also provides methods of using the above-described compositions to induce an immune response against a coronavirus in a subject. In some aspects, the disclosure provides use of any of the above-described immunogenic compositions in the preparation of a medicament, such as a medicament for immunizing an animal against a coronavirus. In other aspects, the disclosure provides a method of inducing an immune response in a subject, the method comprising administering a therapeutically effective amount the above-described coronavirus vaccine, the above-described nanoemulsion, the above-described RIG-I agonist and/or the above-described TLR agonist. In some aspects, the coronavirus vaccine, the nanoemulsion, the agonist of RIG-I, and/or the TLR agonist are present in the same immunogenic composition. In other aspects, the coronavirus vaccine is present in a first composition, and the nanoemulsion, agonist of RIG-I, and/or agonist of a TLR are present in a second composition. Alternatively, each of the coronavirus vaccine, the nanoemulsion, the agonist of RIG-I, and/or the TLR agonist is present individually in separate compositions.

In one aspect, the disclosure relates to a method for vaccination against, or for prophylaxis or therapy (prevention or treatment) of exposure to, or infection with, a coronavirus (such as those described herein) via administration of a therapeutically or prophylactically effective amount of a coronavirus vaccine, a nanoemulsion, a RIG-I agonist, and/or a TLR agonist to a subject in need thereof. Accordingly, administration of the disclosed coronavirus vaccine, nanoemulsion, RIG-I agonist, and/or TLR agonist primes, enables, and/or enhances induction of both humoral (e.g., development of specific antibodies) and cellular (e.g., cytotoxic T lymphocyte) immune responses (e.g., against a coronavirus). Cytokines play a role in directing the immune response. Helper (CD4+) T cells orchestrate the immune response of mammals through production of soluble factors that act on other immune system cells, including B and other T cells. Most mature CD4+T helper cells express one of two cytokine profiles: Th1 or Th2. Th1-type CD4+ T cells secrete IL-2, IL-3, IFN-γ, GM-CSF and high levels of TNF-α. Th2 cells express IL-3, IL-4, IL-5, IL-6, IL-9, IL-10, IL-13, GM-CSF and low levels of TNF-α. Th1 type cytokines promote both cell-mediated immunity, and humoral immunity that is characterized by immunoglobulin class switching to IgG2a in mice and IgG1 in humans. Th1 responses may also be associated with delayed-type hypersensitivity and autoimmune disease. Th2 type cytokines induce primarily humoral immunity and induce class switching to IgG1 and IgE. The antibody isotypes associated with Th1 responses generally have neutralizing and opsonizing capabilities, whereas those associated with Th2 responses are associated more with allergic responses.

Several factors have been shown to influence skewing of an immune response towards either a Th1 or Th2 type response. The best characterized regulators are cytokines. IL-12 and IFN-γ are positive Th1 and negative Th2 regulators. IL-12 promotes IFN-γ production, and IFN-γ provides positive feedback for IL-12. IL-4 and IL-10 appear important for the establishment of the Th2 cytokine profile and to down-regulate Th1 cytokine production.

For example, in some embodiments, the disclosed method results in the skewing of a host's immune response away from Th2 type immune response and toward a Th1 type immune response. In other words, the disclosed methods may induce a cellular immune response that is a Th1-biased immune response. In particular, conventional alum based vaccines for a variety of diseases, such as respiratory syncytial virus (RSV), anthrax, and hepatitis B virus, each lead to a predominant Th2 type immune response in a subject administered the vaccine (e.g., characterized by enhanced expression of Th2 type cytokines and the production of IgG1 antibodies). However, administration of a coronavirus vaccine in combination with the nanoemulsion and RIG-I agonist disclosed herein is able to, in one embodiment, redirect the conventionally observed Th2 type immune response in host subjects administered conventional vaccines. Thus, in some embodiments, the present disclosure provides immunogenic compositions and methods for skewing and/or redirecting a host's immune response (e.g., away from Th2 type immune responses and toward Th1 type immune responses) to one or a plurality of immunogens/antigens. In some embodiments, skewing and/or redirecting a host's immune response (e.g., away from Th2 type immune responses and toward Th1 type immune responses) to one or a plurality of immunogens/antigens comprises providing one or more antigens (e.g., recombinant antigens, isolated and/or purified antigens, antigen-encoding nucleic acid sequences, and/or killed whole pathogens) that are historically associated with generation of a Th2 type immune response when administered to a subject (e.g., a coronavirus antigen).

With respect to viral infections, “humoral immunity” occurs when virus and/or virus-infected cells stimulate B lymphocytes to produce antibody that is specific for viral antigen. IgG, IgM, and IgA antibodies have all been shown to exert antiviral activity. Such neutralizing antibodies can exert antiviral activity by (1) blocking virus-host cell interactions or (2) recognizing viral antigens on virus-infected cells which can lead to antibody-dependent cytotoxic cells (ADCC) or complement-mediated lysis. IgG antibodies are responsible for most antiviral activity in serum, while IgA is the most important antibody when viruses infect mucosal surfaces. In some embodiments, administration of the coronavirus vaccine, nanoemulsion, and RIG-I agonist described herein induces a greater neutralizing antibody response against the coronavirus as compared to administration of the coronavirus vaccine alone.

In some embodiments, the disclosed method reduces the number of booster injections (e.g., of an antigen containing composition) required to achieve a desired immune response (e.g., a protective immune response (e.g., a memory immune response)). In some embodiments, the disclosed method results in a higher proportion of recipients achieving seroconversion and/or more consistent immune responses within a population of subjects administered the immunogenic composition. In some embodiments, the present disclosure provides compositions that are useful for selectively skewing adaptive immunity toward Th1, Th2, or cytotoxic T cell responses (e.g., allowing effective immunization by distinct routes (e.g., such as via mucosa or via injection)). In some embodiments, the present disclosure provides compositions that elicit optimal responses in subjects in which most contemporary vaccination strategies are not optimally effective (e.g., in very young and/or very old populations). Ideally, the disclosed method induces a protective immune response, that is, an immune response that prevents the subject from displaying signs or symptoms of coronavirus infection upon subsequent exposure of the subject to a coronavirus.

In some embodiments, the present disclosure provides compositions that provide efficacy and safety needed for vaccination regimens that involve different delivery routes and elicitation of distinct types of immunity. In some embodiments, the present disclosure provides immunogenic compositions that stimulate antibody responses and have little toxicity and that can be utilized with a range of antigens for which they provide adjuvanticity and the types of immune responses they elicit. In some embodiments, the present disclosure provides immunogenic compositions that meet global supply requirements (e.g., in response to a coronavirus pandemic).

The compositions of the present disclosure can be administered by any suitable route of administration. It will also be appreciated that the chosen route will vary with the condition and age of the recipient, and the disease being treated.

For example, the compositions can be administered by oral, parenteral (e.g., intramuscular, intraperitoneal, intravenous, ICV, intracisternal injection or infusion, subcutaneous injection, or implant), by nasal, vaginal, rectal, sublingual, urethral (e.g., urethral suppository) or topical routes of administration (e.g., gel, ointment, cream, aerosol, etc.), and can be formulated, alone or together, in suitable dosage unit formulations containing conventional non-toxic pharmaceutically acceptable carriers, adjuvants, excipients, and vehicles appropriate for each route of administration. Non-limiting examples of carriers include phosphate buffered saline (PBS), saline or a biocompatible matrix material such as a decellularized liver matrix (DCM as disclosed in Wang et al. (2014) J. Biomed. Mater Res. A. 102(4):1017-1025) for topical or local administration. The compositions can optionally contain a protease inhibitor, glycerol and/or dimethyl sulfoxide (DMSO).

In some embodiments, compositions of the present disclosure are administered mucosally (e.g., using standard techniques; see, e.g., Remington: The Science and Practice of Pharmacy, 23rd Edition, Academic Press (2020) (e.g., for mucosal delivery techniques, including intranasal, pulmonary, vaginal and rectal techniques), as well as European Publication No. 517,565 and Illum et al., J. Controlled Rel., 1994, 29:133-141 (e.g., for techniques of intranasal administration)). Alternatively, the compositions of the present disclosure may be administered dermally or transdermally, using standard techniques (see, e.g., Remington: The Science and Practice of Pharmacy, 23rd Edition, Academic Press (2020)). The present disclosure is not limited by the route of administration.

In some embodiments, the disclosed method is used to protect and/or treat a subject susceptible to, or suffering from, a disease by means of administering the disclosed composition via injection (e.g., via intradermal, subcutaneous, intramuscular, intravenous, intraosseous, intraperitoneal, intrathecal, epidural, intracardiac, intraarticular, intracavernous, and/or intravitreal route). Methods of systemic administration include conventional syringes and needles, or devices designed for ballistic delivery (see, e.g., WO 99/27961), or needleless pressure liquid jet device (see, e.g., U.S. Pat. Nos. 4,596,556 and 5,993,412), or transdermal patches (see, e.g., WO 97/48440 and WO 98/28037). In some embodiments, the present disclosure provides a delivery device for systemic administration, pre-filled with a composition composition of the present disclosure.

In some embodiments, the composition is administered via a mucosal route (e.g., an oral/alimentary or nasal route). Alternative mucosal routes include intravaginal and intrarectal routes. In some embodiments, a nasal route of administration is used, which is also referred to herein as “intranasal administration” or “intranasal vaccination.” Methods of intranasal vaccination are well known in the art, including the administration of a droplet or spray form of a composition into the nasopharynx of a subject to be immunized. Intranasal administration may also include contact with the oral mucosa, bronchial mucosa, and other epithelia. In some embodiments, a nebulized or aerosolized composition is provided. Formulations for nasal delivery may include those with dextran or cyclodextran and saponin as an adjuvant.

Enteric formulations such as gastro-resistant capsules for oral administration and suppositories for rectal or vaginal administration also may be employed. Compositions of the present disclosure may also be administered via the oral route. Under these circumstances, a composition may comprise a pharmaceutically acceptable excipient and/or include alkaline buffers, or enteric capsules. When the composition is administered via a vaginal route, the composition may comprise pharmaceutically acceptable excipients and/or emulsifiers, polymers (e.g., CARBOPOL), and other known stabilizers of vaginal creams and suppositories. When the composition is administered via a rectal route, the composition may comprise excipients and/or waxes and polymers known in the art for forming rectal suppositories.

Oral compositions can be prepared according to methods known in the art, and such compositions may contain one or more agents selected from the group consisting of sweetening agents, flavoring agents, coloring agents, and preserving agents in order to provide pharmaceutically elegant and palatable preparations. Tablets contain active ingredients in admixture with non-toxic pharmaceutically acceptable excipients which are suitable for the manufacture of tablets. These excipients can be for example, inert diluents, such as calcium carbonate, sodium carbonate, lactose, calcium phosphate or sodium phosphate; granulating and disintegrating agents (e.g., corn starch or alginic acid); binding agents (e.g., starch, gelatin, or acacia); and lubricating agents (e.g., magnesium stearate, stearic acid, or talc). The tablets can be left uncoated or they can be coated by known techniques to delay disintegration and absorption in the gastrointestinal tract and thereby provide a sustained action over a longer period. For example, a time delay material such as glyceryl monostearate or glyceryl distearate can be employed. They may also be coated by the techniques described in U.S. Pat. Nos. 4,256,108; 4,166,452; and U.S. Pat. No. 4,265,874 to form osmotic therapeutic tablets for control release. The pharmaceutical compositions of the disclosure may also be in the form of oil-in-water emulsions.

Liquid preparations for oral administration may take the form of, for example, elixirs, solutions, syrups, or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives, or hydrogenated edible fats); emulsifying agents (e.g., lecithin, or acacia); non-aqueous vehicles (e.g., almond oil, oily esters, ethyl alcohol, Cremophore™, or fractionated vegetable oils); and preservatives (e.g., methyl or propyl-p-hydroxybenzoates or sorbic acid). The preparations may also contain buffer salts, preservatives, flavoring, coloring, and sweetening agents as appropriate.

When the coronavirus vaccine, the nanoemulsion, the RIG-I agonist, and/or the TLR agonist are separately formulated into different compositions, each composition may be administered via the same administration route or via multiple different administration routes. For example, a composition comprising the coronavirus vaccine may be administered intramuscularly, and compositions comprising the nanoemulsion and the RIG-I agonist and/or TLR agonist may be administered intranasally. Alternatively, a composition comprising the coronavirus vaccine may be administered intramuscularly, a composition comprising the nanoemulsion may be administered intranasally, and a composition comprising the RIG-I agonist and/or TLR agonist may be administered subcutaneously. In other embodiments, a composition comprising the coronavirus vaccine and the RIG-I agonist and/or TLR agonist may be administered intramuscularly, and a composition comprising the nanoemulsion may be administered intranasally.

In some embodiments, the same route of administration (e.g., mucosal administration) is chosen for both a priming and boosting vaccination. In other embodiments, multiple routes of administration are utilized (e.g., at the same time, or, alternatively, sequentially) in order to stimulate an immune response (e.g., using one or more compositions of the present disclosure).

In some embodiments, the disclosed composition(s) is/are administered to a mucosal surface of a subject in either a priming or boosting vaccination regime. Alternatively, in some embodiments, a composition is administered systemically in a priming and/or boosting vaccination regime. In some embodiments, an immunogenic composition is administered to a subject in a priming vaccination regimen via mucosal administration and a boosting regimen via systemic administration. In some embodiments, an immunogenic composition is administered to a subject in a priming vaccination regimen via systemic administration and a boosting regimen via mucosal administration. Examples of systemic routes of administration include, but are not limited to, a parenteral, intramuscular, intradermal, transdermal, subcutaneous, intraperitoneal or intravenous administration.

Although an understanding of the mechanism is not necessary to practice the present disclosure and the present disclosure is not limited to any particular mechanism of action, in some embodiments, the nanoemulsion and RIG-I agonist and/or TLR agonist acts to transport and/or present antigen/immunogen (e.g., a coronavirus antigen) to the immune system (e.g., to antigen presenting cells of the immune system). In this regard, for example, the nanoemulsion in combination with a RIG-I agonist and/or TLR agonist acts to transport and/or present antigen (e.g., a coronavirus antigen) to the immune system (e.g., to antigen presenting cells of the immune system) in a greater way or in a synergistic way compared to when the one or more antigens are administered with only the nanoemulsion (e.g., a nanoemulsion described herein) or alone. In some embodiments, mucosal administration of an immunogenic composition of the present disclosure generates mucosal (e.g., signs of mucosal immunity (e.g., generation of IgA antibody titers)) as well as systemic immunity. In some embodiments, mucosal administration of the composition of the disclosure generates an innate immune response (e.g., activates Toll-like receptor signaling and/or activation of NF-kB) in a subject. Both cellular and humoral immunity play a role in protection against multiple pathogens and both can be induced with the composition of the present disclosure.

In some embodiments, the composition may be applied and/or delivered utilizing electrophoretic delivery/electrophoresis. Further, compositions may be applied by a transdermal delivery system such as a patch or administered by a pressurized or pneumatic device (i.e., “gene gun”). Such methods, which comprise applying an electrical current, are well known in the art.

The compositions described herein may be administered topically. If applied topically, the compositions may be occluded or semi-occluded. Occlusion or semi-occlusion may be performed by overlaying a bandage, polyoleofin film, article of clothing, impermeable barrier, or semi-impermeable barrier to the topical preparation.

The pharmaceutical compositions for administration may be applied in a single administration or in multiple administrations. Indeed, as discussed above, following an initial administration of a composition of the present disclosure (e.g., an initial vaccination), a subject may receive one or more boost administrations (e.g., around 2 weeks, around 3 weeks, around 4 weeks, around 5 weeks, around 6 weeks, around 7 weeks, around 8 weeks, around 10 weeks, around 3 months, around 4 months, around 6 months, around 9 months, around 1 year, around 2 years, around 3 years, around 5 years, around 10 years) subsequent to a first, second, third, fourth, fifth, sixth, seventh, eighth, ninth, tenth, and/or more than tenth administration. Although an understanding of the mechanism is not necessary to practice the present disclosure and the present disclosure is not limited to any particular mechanism of action, in some embodiments, reintroduction of an immunogen in a boost dose enables vigorous systemic immunity in a subject. The boost can be with the same formulation given for the primary immune response, or can be with a different formulation that contains the immunogen. The dosage regimen will also, at least in part, be determined by the need of the subject and be dependent on the judgment of a practitioner.

Dosage units may be proportionately increased or decreased based on several factors including, but not limited to, the weight, age, and health status of the subject. In addition, dosage units may be increased or decreased for subsequent administrations (e.g., boost administrations).

It is contemplated that the compositions and methods of the present disclosure will find use in various settings, including research settings. For example, compositions and methods of the present disclosure also find use in studies of the immune system (e.g., characterization of adaptive immune responses (e.g., protective immune responses (e.g., mucosal or systemic immunity))). Uses of the compositions and methods provided by the present disclosure encompass human and non-human subjects and samples from those subjects, and also encompass research applications using these subjects. Compositions and methods of the present disclosure are also useful in studying and optimizing nanoemulsions, immunogens, and other components and for screening for new components. Thus, it is not intended that the present disclosure be limited to any particular subject and/or application setting.

In some embodiments, the present disclosure provides a kit comprising a coronavirus vaccine, a nanoemulsion, a RIG-I agonist, and/or TLR agonist, and/or compositions comprising same. In some embodiments, the kit further contains a device for administering compositions. The present disclosure is not limited by the type of device included in the kit. In some embodiments, the device is configured for nasal application of a composition of the present disclosure (e.g., a nasal applicator (e.g., a syringe) or nasal inhaler or nasal mister). In some embodiments, a kit comprises the coronavirus vaccine, the nanoemulsion, the RIG-I agonist, and/or the TLR agonist in concentrated form (e.g., that can be diluted prior to administration to a subject).

In some embodiments, all kit components are present within a single container (e.g., vial or tube). Alternatively, each kit component may be located in a single container (e.g., vial or tube). In other embodiments, one or more kit components are located in a single container (e.g., vial or tube) with other components of the same kit being located in a separate container (e.g., vial or tube). In some embodiments, the kit comprises a buffer. The kit may further comprise instructions for use.

The following examples further illustrate the disclosure but, of course, should not be construed as in any way limiting its scope.

### EXAMPLES

Materials and Methods Adjuvants and antigen

A nanoemulsion (NE) was produced by emulsification of cetylpyridinium chloride (CPC) and Tween 80 surfactants, ethanol (200 proof), super refined soybean oil (Croda) and purified water using a high speed homogenizer as previously described. CPC and Tween80 were mixed at a 1:6 (w/w) ratio, and homogeneity of particle size (d=450-550 nm) and charge (zeta potential=50-55 mV) were confirmed. Stability was assessed over several months. Sequence and synthesis methods for IVT DI RNA have previously been reported in detail. Briefly, SeV DI RNA from SeV-infected A549 cells was amplified using a 5′ primer with the T7 promoter and a 3′ primer with the hepatitis delta virus genomic ribozyme site followed by the T7 terminator. The resultant DNA was cloned into a pUC19 plasmid and in vitro transcribed using a HiScribe T7 High Yield RNA synthesis kit (New England Biolabs). After DNAseI digestion and clean-up with a TURBO DNA-free kit (Thermo-Fisher), IVT DI was purified using an RNeasy purification kit (Qiagen). The absence of endotoxin was verified by limulus amoebocyte lysate assay. Recombinant SARS-CoV-2 spike protein S1 subunit (Wuhan-Hu-1 (Val16-Arg685) (accession YP_009724390.1)) with a C-terminal His tag was purchased from Sino Biological.

### Cell Lines

Vero E6 cells (ATCC) were maintained in MEM supplemented with 10% heat inactivated fetal bovine serum (HI FBS). HEK293T cells expressing hACE2 (293T-hACE2) were obtained from BEI resources and maintained in HEK293T medium: DMEM containing 4 mM L-glutamine, 4500 mg/L L-glucose, 1 mM sodium pyruvate and 1500 mg/L sodium bicarbonate, supplemented with 10% HI FBS as previously described.

### Viruses

WT SARS-CoV-2: SARS-CoV-2 clinical isolate USA-WA1/2020 (BEI resources; NR-52281), referred to as the WT virus herein, was propagated by culture in Vero E6 cells as previously described⁷⁵. MA SARS-CoV-2: Mouse-adapted SARS-CoV-2 was obtained by serial passage of the USA-WA1/2020 clinical isolate in mice of different backgrounds over eleven passages, as well as on mACE2 expressing Vero E6 cells as previously described⁵⁰. Briefly, the virus was passaged every two days via IN inoculation with lung homogenate derived supernatants from infected mice. All viral stocks were analyzed by deep sequencing to verify integrity of the original viral genome.

### Lentivirus Pseudotyped Virus

Cloning of expression constructs: For generation of spike protein pseudotyped lentivirus (Lenti-CoV2), a codon optimized SARS-CoV-2 spike protein (accession #QHD43416.1) construct was obtained from Sino Biologicals. All cloning and lentivirus production was performed by the University of Michigan Vector Core. The SARS-CoV-2 spike with 19 amino acids deleted (SΔ19) was generated by PCR amplifying the region of spike containing amino acids 738 to 1254 from the full length SARS-CoV-2 spike construct. The product was cloned into a pCMV3 vector digested at the BsrGI/XhoI sites. Resulting clones were verified by Sanger sequencing. The resulting clone was designated pCMV3-SA19. For the generation of a lentiviral vector containing SARS-CoV2-SpikeΔ19, the pCMV3-SA19 insert was initially digested with KpnI and blunt polished using Phusion Taq polymerase followed by a DNA cleanup using the Monarch PCR cleanup kit (NEB) and a second digest was done using NotI. The released fragment was then ligated into a pLentiLox-RSV-CMV-Puro vector. Correct insertion was verified by Sanger sequencing. The resulting clone was designated pSARsCoV2Δ19AA.

To prepare Lenti-CoV-2 pseudovirus expressing the SARS-CoV-2 S protein, lentivirus packaging vectors psPAX2 (35 μg), and coronavirus truncated spike envelope pSARsCoV2Δ19AA (35 μg) were co-transfected with 70 μg of pGF1-CMV proviral plasmid using standard PEI precipitation methods. PEI precipitation was performed by incubating the plasmids with 420 μg PEI (molecular weight 25,000, Polysciences, Inc) in 10 mL Opti-MEM (Life technologies) at room temp for 20 m, before adding to fresh 90 mL of DMEM media supplemented with 10% FBS-1×Glutamax-100 U/mL Penn/Strep. This DNA/PEI containing media was then distributed equally to 5-T150 flasks (Falcon) containing 293T cells. Supernatants were collected and pooled after 72 h, filtered through a 0.45 μm GP Express filter flask (Millipore), pelleted by centrifugation at 13,000 rpm on a Beckman Avanti J-E centrifuge at 4° C. for 4 h, and resuspended at 100× the original concentration (˜1×10⁷ TU/ml) in DMEM. Harvested lentivirus was stored at −80 C.

### Animals

All animal procedures were approved by the Institutional Animal Care and Use Committees (IACUC) at the University of Michigan and Icahn School of Medicine at Mt. Sinai and were carried out in accordance with these guidelines. 6-8-wk-old female C57Bl/6 mice (Jackson Laboratory) were housed in specific pathogen-free conditions. Mice were acclimated for 2 weeks prior to initiation. For challenge studies, mice were transferred to ABSL3 facilities 2 d prior to serum transfer and subsequent viral challenge.

### Immunization

For intranasal (IN) immunization, mice were anesthetized under isoflurane using an IMPAC6 precision vaporizer and given 12 μL total (6 μL/nare) of each vaccination mixture. Each group received a total of three immunizations of the same formulations at 4-wk intervals. 15 μg of S1 was administered with either PBS, 20% NE (w/v), or 20% NE with 0.5 μg of IVT DI in PBS. Sera were obtained by saphenous vein bleeding 2 and 4 weeks after each immunization, and by cardiac puncture at the end of the experiment at week 10. Bronchial alveolar lavage (BAL) was obtained by lung lavage with 0.8 mL PBS containing protease inhibitors. Spleens and cervical lymph nodes were harvested, processed to single-cell suspensions, and cultured for antigen recall response assessment as previously described.

### ELISA

Immunograde 96-well ELISA plates (Midsci) were coated with 100 ng S1 in 50 μL PBS per well overnight at 4° C., and then blocked in 200 μL of 5% non-fat dry milk/PBS for 1 h at 37° C. Serum samples from immunized mice were serially diluted in PBS/0.1% BSA starting at either a dilution of 1:50 or 1:100. Blocking buffer was removed, and diluted sera were added to the wells and incubated for 2 h at 37° C. followed by overnight incubation at 4° C. Plates were washed three times with PBST (0.05% Tween20), and alkaline phosphatase conjugated secondary antibodies diluted in PBS/0.1% BSA were added (goat-anti-mouse IgG, IgG1, IgG2b, or IgG2c Jackson Immuno Research Laboratories). Plates were incubated at 37° C. for 1 h, washed with PBST, and then developed at RT by addition of 100 μL of p-nitrophenyl phosphate (pNPP) substrate (Sigma-Aldrich) per well. Absorbance was measured at 405 nm on a microplate spectrophotometer. Titers were calculated against naïve sera, using a cutoff value defined by the sum of the average absorbance at the lowest dilution and two times the standard deviation.

### Pseudovirus Microneutralization (MNT) Assays

9×10³ 293T-hACE2 cells were seeded overnight on white clear bottom 96-well tissue culture plates in HEK293T medium. To titer the virus, the Lenti-Cov2 stock was serially diluted in HEK293T medium with 16 μg/mL polybrene (Sigma-Aldrich), and incubated for 1 h at 37° C. to mimic assay conditions for MNT. Diluted virus was then added to the 293T-hACE2 cells and incubated at 37° C. for 4 h. The media was replaced with fresh HEK293T medium without polybrene and incubated for an additional 72 h at 37° C. Infection medium was removed and replaced with 20 μL of BrightGlo luminescence reagent using an injection luminometer. Cells were incubated for 2 m with shaking and luminescence was collected over a read time of 1 s. For MNT, 293T-hACE2 cells were seeded overnight. Serum samples from immunized mice were serially diluted by a factor of two, starting at a dilution of 1:50 in HEK293T medium with 16 μg/mL polybrene (Sigma-Aldrich). 50 μL of diluted sera was added to 50 μL of lenti-CoV2 in the same media at a concentration which gave a luminescence reading of ≥20,000 RLU/well above background in infected cells as determined by viral titration. Serum and virus were incubated for 1 h at 37° C., and then added to 293T-hACE2 cells for incubation at 37° C. for 4 h. Infection medium was removed, and replaced with fresh HEK293T medium without polybrene and incubated for an additional 72 h at 37° C. Luminescence was measured as above. Neutralization titers were determined as the dilution at which the luminescence remained below the luminescence of the (virus only control-uninfected control)/2.

### Microneutralization Assays

MNT assays with WT SARS-CoV-2 (2019-nCoV/USA-WA1/2020) and the mouse adapted (MA-SARS-CoV-2) variant was performed in a BSL3 facility as previously described. Briefly, 2×10⁴ Vero E6 cells were seeded per well in a 96-well tissue culture plate overnight. Serum samples were heat-inactivated for 30 m at 56° C. and serially diluted by a factor of 3, starting at dilutions of 1:10 or 1:20 in infection medium (DMEM, 2% FBS, 1× non-essential amino acids). Diluted serum samples were incubated with 450×TCID50 of each virus which (˜40 PFU) for 1 h at 37° C. Growth medium was removed from the Vero E6 cells, and the virus/serum mixture was added to the cells. Plates were incubated at 37° C. for 48 h, fixed in 4% formaldehyde, washed with PBS and blocked in PBST (0.1% Tween 20) for 1 h at RT. Cells were permeabilized with 0.1% TritonX100, washed and incubated with anti-SARS-CoV-2-nucleoprotein and anti-SARS-CoV-2-Spike monoclonal antibodies, mixed in 1:1 ratio, for 1.5 h at RT. After another wash, cells were incubated with HRP-conjugated goat-anti-mouse IgG secondary antibody for 1 h at RT. Cells were washed, and plates were developed by incubation with 50 μL tetramethyl benzidine until a visible blue color appeared, after which the reaction was quenched by addition of 50 μL 1M H₂SO₄. Absorbance was measured at 450 nm and percentage inhibition (reduction of infection) was calculated against virus only infected controls. The 50% inhibitory dilution (IC50) values were calculated for each sample. Undetectable neutralization was designated as a titer of 10⁰. Anti-mouse SARS-CoV-2-nucleoprotein and anti-mouse SARS-CoV-2-spike antibodies were obtained from the Center for Therapeutic Antibody Development at the Icahn School of Medicine at Mount Sinai.

### Antigen Recall Response

T cell antigen recall response was assessed in cell isolates from the spleen and cLN of immunized mice 2 weeks after the final immunization (week 10). Methods for splenocyte and cLN lymphocyte preparation were previously described. For antigen recall, isolated cells were plated at a density of 8×10⁵ cells/well and stimulated with 5 μg per well of recombinant S1 in T cell media (DMEM, 5% FBS, 2 mM L-glutamine, 1% NEAA, 1 mM sodium pyruvate, 10 mM MOPS, 50 μM 2-mercaptoethanol, 100 IU penicillin, and 100 μg/mL streptomycin), in a total volume of 200 μL per well. Cells were stimulated for 72 h at 37° C., and secreted cytokines (IFN-γ, IL-2, IL-4, IL-5, IL-6, IL-17A, TNF-α, and IP-10) were measured relative to unstimulated cells in supernatants using a Milliplex MAP Magnetic Mouse Cytokine/Chemokine multiplex immunoassay (EMD Millipore).

### Acute Cytokine Response

Acute response markers were measured in immunized mice 6 and 12 h post-initial immunization to determine whether the formulations induced reactogenicity. For each group, mice were bled either at 6 or 12 h and the amounts of IFNγ, IL6, IL12p70, and TNFα in the sera were measured using a Procartaplex multiplex immunoassay (ThermoFisher) according to the manufacturer's protocol.

### Passive Transfer and Challenge

Serum samples were pooled from mice in each immunization group collected after the second boost immunization (week 10), and 150 μL of the pooled serum was passively transferred into naïve mice through the intraperitoneal route 2 h prior to challenge intranasally under mild ketamine/xylazine sedation with 10⁴ PFU of MA-SARS-CoV-2 in 30 μL. Body weight changes were recorded every 24 h, and mice were sacrificed at 3 d.p.i. Lungs were harvested and homogenate prepared for virus titration by plaque assay as previously described.

### Avidity

To measure antibody avidity of serum IgG in immunized mice, ELISAs were performed as described above against S1 modified by an additional chaotrope elution step after the overnight incubation of serum on the ELISA plate as previously described. Briefly, after the serum incubation and washes with PBST, 100 μL of PBS, or 0.5 M NaSCN, or 1.5 M NaSCN in PBS at pH 6 was added to each well and incubated at RT for 15 min. The plates were washed three times with PBST, and the ELISA proceeded to development as described above by addition of alkaline phosphatase conjugated goat-anti-mouse IgG.

### Example 1

This example describes a safety and acute response evaluation of the method disclosed herein.

An NE adjuvant as described herein has completed phase I testing in humans as an intranasal (IN) adjuvant and is currently in another ongoing phase I trial. Extensive characterization of this adjuvant has been performed in multiple animal models, each demonstrating optimal safety profiles for use of the adjuvant through both intramuscular and IN routes. However, to ensure that simultaneously stimulating TLRs, NLRs, and RIG-I with NE/IVT DI does not lead to over-activation of an inflammatory response, the acute cytokine response of the NE adjuvant was evaluated by assessing the levels of representative cytokines (IL-6, TNF-α, IL12p70, and IFN-γ) in the serum with a multiplex immunoassay at 6 and 12 hours post-immunization using a model antigen. SARS-CoV-2 RBD was used as a model antigen.

Mice were immunized through the IN route with 10 μg of RBD alone, or combined with the standard doses of NE (20% w/v) or NE/IVT DI (20% NE/0.5 μg IVT DI), used in all subsequent studies discussed below in a total volume of 12 μL PBS per mouse. Minimal or no acute inflammatory cytokines were detectable in the sera at these time points, with only IL-6 being modestly elevated in the NE/RBD (mean 34.4 μg/mL) and NE/IVT DI/RBD (mean 74.7 μg/mL) groups, as compared to the RBD only group (mean 11.6 μg/mL). No significant amounts of TNF-α, IL12p70, and IFN-γ were detectable.

These results are consistent with the safety profile of the NE alone, supporting a lack of systemic toxicity with the NE/IVG DI combination. These results are also consistent with the lack of significant changes in body temperature or weight in NE/IVT DI immunized mice in previous studies with influenza virus antigens (data not shown).

### Example 2

This example describes the characterization of the humoral response induced by immunization with SARS-CoV-2 S1.

To assess the impact of the combined NE/IVT DI adjuvant on the humoral response, the S1 subunit of the full length SARS-CoV-2 spike protein was chosen as the antigen for immunization. The S1 subunit was chosen because it contains the S protein receptor binding domain (RBD), which is necessary for interacting with the human ACE2 receptor (hACE2) required for viral entry, and thus contains the key epitopes necessary for neutralizing antibody recognition. Furthermore, previous studies evaluating vaccine candidates for related SARS-CoV have demonstrated that the S1 subunit induces a comparable humoral immune response as the full-length S protein, while avoiding the problems of vaccine associated enhanced respiratory disease (VAERD) and antibody dependent enhancement (ADE) induced by some vaccines containing native full-length S protein. On the other hand, recombinant SARS-CoV-2 RBD alone has been found to be less immunogenic than the S1 and full length S protein. Moreover, by providing these additional antigenic sites outside of the RBD by immunizing with the S1 subunit rather than the RBD itself, it is possible that improved crossvariant protective immunity may be achieved.

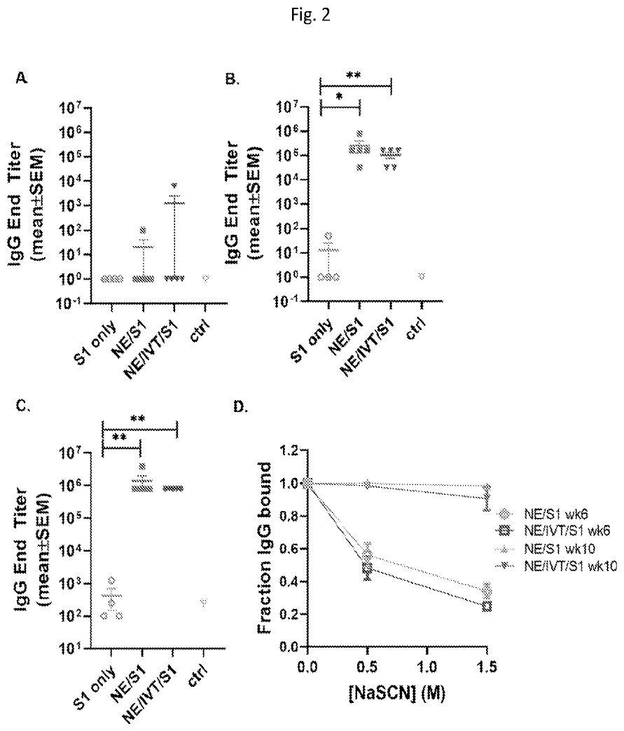

6-8 wk old C57Bl/6 mice were immunized IN with 15 μg of SARS-CoV-2 S1 alone (S1 only), with 20% NE (NE/S1), or with 20% NE/0.5 μg IVT DI (NE/IVT/S1) according to a prime/boost/boost schedule at a four-week interval. Serum S1-specific total IgG titers were measured two weeks after each immunization (FIG. 2). After one immunization, no detectable antigen-specific IgG was observed in the S1 only immunized mice or in the majority of mice immunized with the adjuvants: NE/S1 and NE/IVT/S1 (FIG. 2A). However, each adjuvant group had a high responder which displayed detectable S1-specific IgG, displaying a titer of 1:100 for NE/S1, and a titer of 1:6250 for the NE/IVT/S1 group, suggesting the possibility for an improved synergistic effect in early antibody titers with the combined adjuvant upon further optimization of antigen dose. S1-specific IgG increased significantly in both adjuvanted groups after the second immunization, resulting in comparable geomean IgG titers (GMTs) in the range of ˜10⁵ for the NE/S1 and NE/IVT/S1 groups, respectively (FIG. 2B), which were further enhanced by the third immunization to titers of ˜10⁶ (FIG. 2C). In contrast, the S1 only group showed minimal IgG even after the second immunization, and reached only a mean titer of 1:200 after the third immunization.

As it has been shown that the combined adjuvant significantly enhances the avidity of antigen-specific IgG from immunized mice to whole influenza virus, it was examined whether the combined adjuvant also improved the avidity of the induced S1-specific antibodies. Avidity was measured by chaotrope elution of serum S1-specific IgG measured after the second and third immunizations using 0.5 M, and 1.5 M NaSCN in PBS pH 6 (FIG. 2D). The NE/S1 and NE/IVT/S1 groups displayed identical antibody avidity for S1 at week 6, which was significantly enhanced in both groups after the last immunization (week 10). Both NE and NE/IVT DI induced very high affinity antibodies after three immunizations, with 95-100% of the S1-specific IgG remaining bound even upon elution with a high (1.5M) concentration of NaSCN and even at the high dilution of serum tested (11,250). Such strong avidity was significantly greater than that of the antibodies evaluated from convalescent COVID-19 patient sera, which had only 20-60% of the S1 antibodies remaining bound under a less stringent elution condition of 1 M NaSCN.

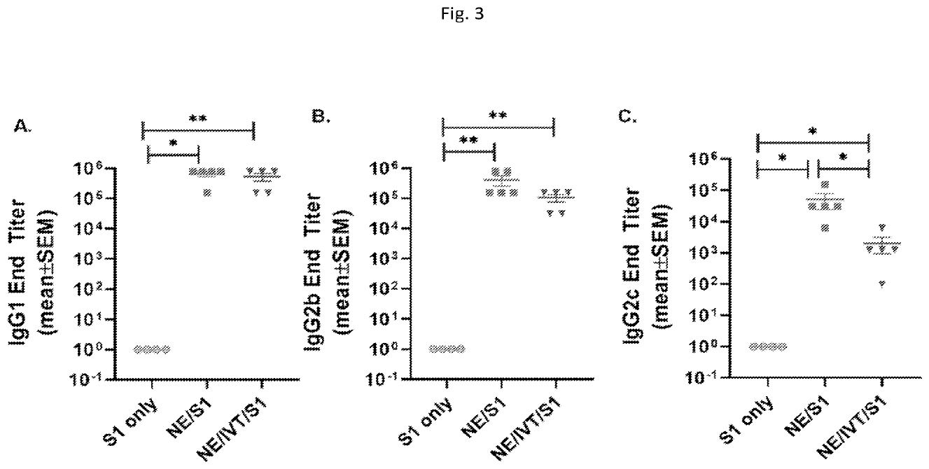

To examine the IgG subclass distribution induced by the NE and the combined NE/IVT DI adjuvant, the relative titers of IgG1, IgG2b, and IgG2c were measured for the week 10 sera (FIG. 3). Subclass analysis suggests that a balanced TH1/TH2 profile was elicited for both the NE and NE/IVT DI adjuvants, as has been observed in previous studies with other antigens. Equivalent titers of IgG1 were induced by both NE and NE/IVT DI with GMTs of 5.7×10⁵ and 4.1×10⁵, respectively (FIG. 3A). High titers of TH1 associated subclasses were also observed for both the NE and NE/IVT DI. SI-specific IgG2b GMTs of 3.0×10⁵ and 8.2×10⁴ (FIG. 3B), and IgG2c GMTs of 3.1×10⁴ and 1.0×10³ were observed for NE and NE/IVT DI adjuvanted groups, respectively (FIG. 3C). Interestingly, IgG2 subclasses were slightly reduced by the presence of IVT DI in the adjuvant. This is distinct from what was observed in previous studies using this combined adjuvant with whole inactivated influenza virus as the antigen, in which the presence of IVT DI and other RIG-I activating RNA agonists significantly increased the amount of IgG2 subclasses relative to the NE alone. These results may suggest that these subtle differences are due to the dissimilarities in immunization with a whole inactivated virus containing additional PAMPs, versus the purified recombinant S1 protein. While it is still unclear whether ADCC and ADCP have a prominent role in SARS-CoV-2 immunity, as is the case for influenza virus, the prevalence of antigen-specific IgG2b and 2c antibody subclasses is promising for these other modes of protective immunity outside of antibody neutralization. Importantly, this balanced TH1/TH2 profile in combination with the cytokine data presented below suggest that these adjuvants avoid the strongly TH2-biased immune responses that have previously been linked to VAERD in SARS-CoV and RSV vaccine candidates adjuvanted with alum.

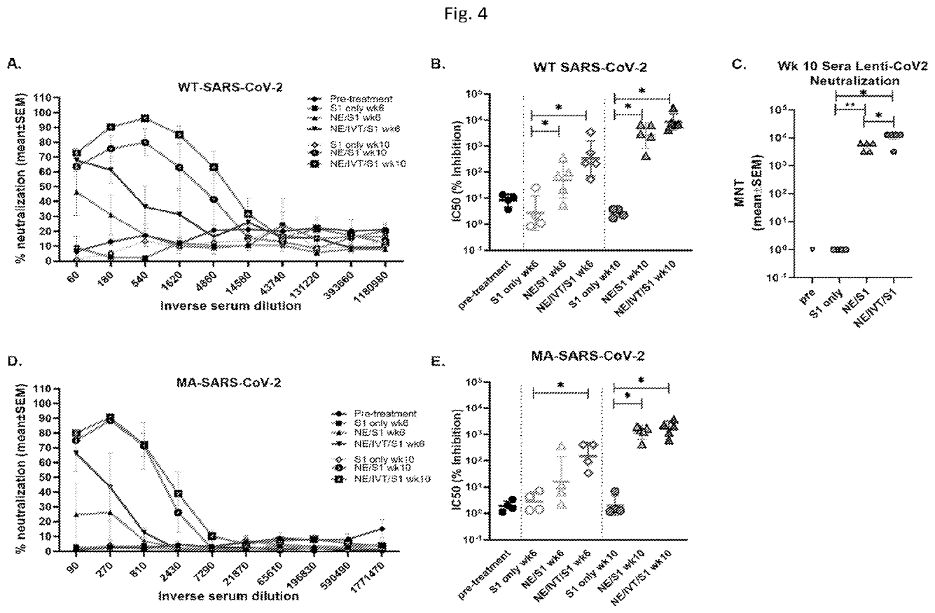

To examine the functionality of the induced antibodies, viral neutralization was assessed using virus stock prepared from the clinical isolate, 2019-nCoV/USA-WA1/2020, which is referred to herein as “WT” SARS-CoV-2 (FIG. 4A, B). The WT SARS-CoV-2 shares a high degree of homology with the Wuhan-Hu-1 isolate from which the S1 subunit used for immunization was derived. To examine the ability of the antibodies from the immunized mice to neutralize a variant with mutations in the S protein, the neutralization assay was repeated with a mouse-adapted SARS-CoV-2 virus (MA-SARS-CoV-2). The MA-virus was generated by serial passaging the WT virus isolate first in the lungs of immune compromised mice, and then in immune competent mice of different backgrounds to optimize mouse virulence, as previously described. As the WT virus is unable to use the endogenously expressed mouse ACE2 receptor (mACE2) for entry, productive infection of the murine respiratory tract is inefficient. Serial passaging allowed for selection of mutations which allowed the virus to adapt to optimally bind and use mACE2 for infection. In the spike protein, the MA-SARS-CoV-2 contains two amino acid mutations compared to the original WT virus from which it was derived, including N501Y and H655Y, and a four amino acid insertion within the S1 subunit as previously described. The N501Y mutation has previously been reported in an independent mouse adaptation of SARS-CoV-2, and is thus likely to be important for increasing affinity to the mACE2 receptor. Interestingly, the N501Y mutation is shared by both the recently identified circulating variants, B1.1.7 and B.135.1, and is thought to play a role in the increased human to human transmissibility observed for these variants by increasing the affinity of the S protein for the hACE2 receptor. In addition to the changes in the spike protein, the MA-virus also contains three other mutations when compared to the Wuhan-Hu-1 isolate: S194T in the nucleoprotein, T7I in the M protein, and L84S in ORF8. The L84S mutation is present in the USA-WA1/2020 strain, however, and is most likely not due to mouse adaptation.

As the WT and MA viruses require the use of BSL-3 containment facilities, to facilitate future vaccine candidate screening, a luciferase-based pseudotyped virus assay was validated. A lentivirus pseudotyped virus expressing the SARS-CoV-2 S protein from the same variant from which the S1 subunit used for immunization was derived (Wuhan-Hu-1), was constructed (Lenti-CoV2), carrying genes for firefly luciferase as described above. The S protein on Lenti-CoV2 contains amino acid (aa) residues 738-1254 of the full length S protein, including a terminal 19 aa deletion which removes an ER retention signal, which has been shown to facilitate the generation of spike pseudotyped lentivirus.

Microneutralization assays using the WT-SARS-CoV-2 with sera from mice immunized with S1 alone revealed very low or undetectable neutralizing antibody titers, similar to naïve mice after three immunizations (FIG. 4A, 4B). In contrast, mice immunized with NE/S1 showed viral neutralization titers (IC50 GMT 50; range 5-353) after the first boost immunization (week 6), which was further increased by two orders of magnitude (IC50 GMT 2.6×10³; range 0.4-6.8×10³) after the second boost immunization (week 10). The combined adjuvant enhanced neutralization titers compared to the NE alone. Sera from mice immunized with NE/IVT DI/S1 showed increased IC50 values approximately an order of magnitude higher than the NE/S1 group, giving an IC50 GMT of 340 (range 52 to 3.5×10³) and IC50 GMT of 8.6×10³ (range 4.3×10³-3×10⁴) after the first and second boosts, respectively. Interestingly, this enhancement in virus neutralization was observed with the combined adjuvant, even though there were no differences observed in either the total IgG titers or IgG avidity between the NE/S1 group and the NE/IVT/S1 group at either time point. Notably, the sera from NE/IVT/S1 immunized mice reached a maximum of ˜100% viral neutralization after the last immunization, This level of neutralization was maintained down to serum dilutions often reported as the IC50 in a large proportion of human convalescent serum and for antibodies induced by some lead vaccine candidates in humans and in mice. While it is difficult to directly extrapolate results, as neutralization assays still need to be standardized for SARS-CoV-2, these results support induction of high-quality antibodies with the combined adjuvant.

Neutralization titers were confirmed using the Lenti-CoV2 pseudotyped virus in a luciferase based assay with 293T-hACE2 cells (FIG. 4C). Microneutralization titers (MNTs) determined by measuring reduction in luminescence (viral infection) with the pseudovirus at week 10 showed almost exact correlation with the traditional microneutralization assay with the WT virus. No neutralizing antibodies were detectable with this method for the S1 only group, and a similar degree of enhancement in the MNT (˜an order of magnitude increase) was observed for the combined adjuvant compared to the NE alone, as was seen in the traditional microneutralization assay.

While slightly reduced compared to titers for WT-SARS-CoV-2 and Lenti-CoV2, high cross-variant neutralization titers were still observed when the sera from NE/IVT DI immunized mice were tested against the MA-SARS-CoV-2 (FIG. 4D, E). After two immunizations, the difference in enhancement in neutralization by sera from NE/IVT DI immunized mice (IC50 GMT 150; range 34-405) compared to NE alone (IC50 GMT 16; range 2.2-360) appeared to be greater due to the larger drop in the ability of the sera from NE immunized mice to cross-neutralize the MA-virus. However, after the third immunization, differences between the NE and NE/IVT DI groups became similar to the differences observed with the WT virus, as the neutralization titers of both groups increased (IC50 GMT 1.2×10³; range 420 to 1.9×10³ for NE/S1), (IC50 GMT 1.6×10³; range 608 to 3.7×10³ for NE/IVT/S1).

The results of this example suggest that the combined adjuvant may strengthen the quality of the antibody response, providing a protective advantage against divergent variants.

### Example 3

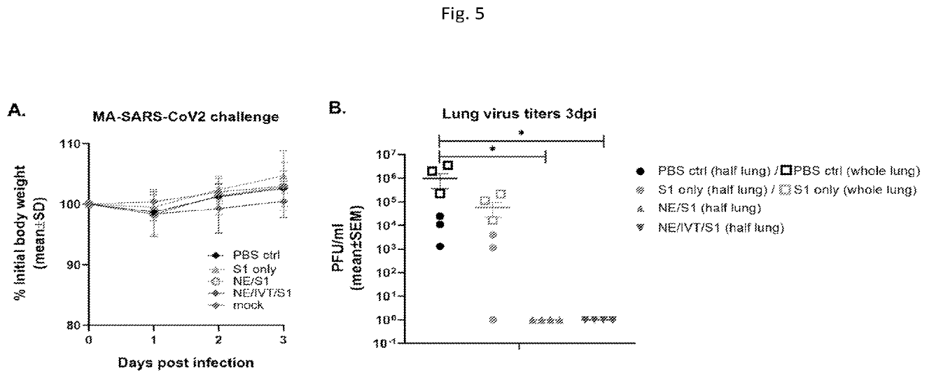

Neutralizing antibody (Nab) titers required for protection against SARS-CoV-2 have yet to be determined. However, studies in non-human primates (NHPs) suggest that low titers (1:50) administered prior to challenge are enough to impart partial protection from a low dose viral challenge, whereas titers of 1:500 conferred full protection to the homologous virus. To determine whether the antibodies raised against the S1 of Wuhan-Hu-1 could protect against heterologous challenge with MA-SARS-CoV-2, week 10 sera from immunized mice were pooled and passively transferred into naïve mice intraperitoneally before challenging IN with 10⁴ PFU virus. While the MA-virus causes mortality and morbidity in aged mice, young C57Bl/6 mice do not lose body weight in this challenge model, as was the case in this study (FIG. 5A). None of the mice receiving serum from immunized animals displayed changes in body weight or increased illness, which also suggests an absence of antibody dependent enhancement (ADE) of disease. Lungs from challenged mice were harvested at 3 d.p.i. for measurement of viral titers in homogenate by plaque assay (FIG. 5B). Mice receiving transferred sera from NE/S1 and NE/IVT/S1 groups showed complete sterilizing protection against challenge, with no detectable virus in the lungs. Given the high NAb titers present in both the single and combined adjuvant groups at week 10, it is not surprising that challenge with this moderate viral dose shows no differences between the groups. In contrast, only a slight reduction in viral titers was found for two out of three mice receiving sera from the S1 only immunized mice as compared to the group receiving no serum (PBS control), and one animal in the S1 only group had no viral titers after challenge. The passive transfer was repeated for the PBS control and S1 groups, but this time, virus titers were determined in the whole lung in the repeated challenge. High titers of virus are observed in all of the mice receiving the S1 only sera when whole lung is considered, and again a slight reduction (˜10-fold) is observed when compared to whole lung homogenate of mice receiving no serum. This is consistent with the presence of low levels of NAbs in the S1 serum.

### Example 4

This example describes the antigen-specific cellular response profile of immunized mice.

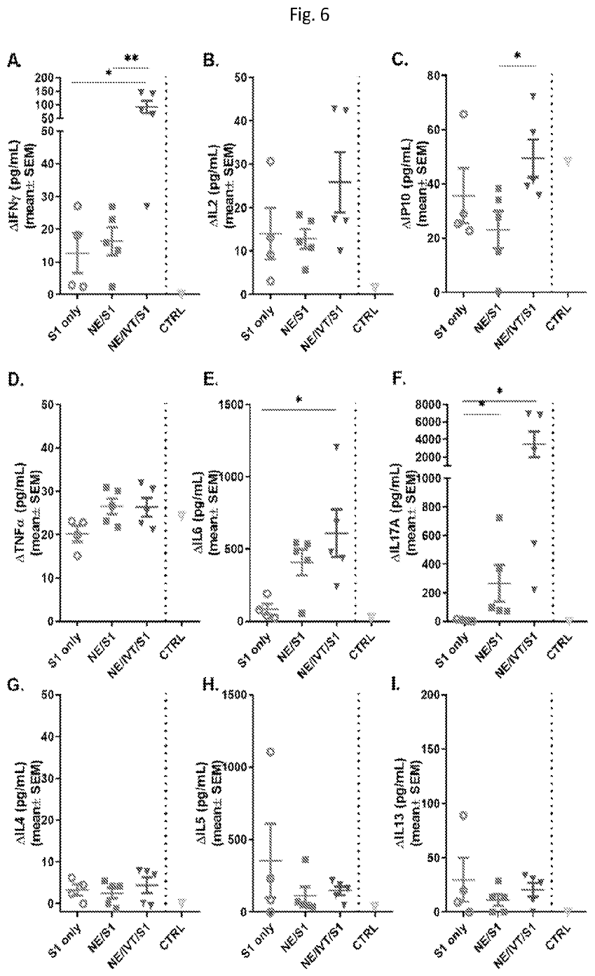

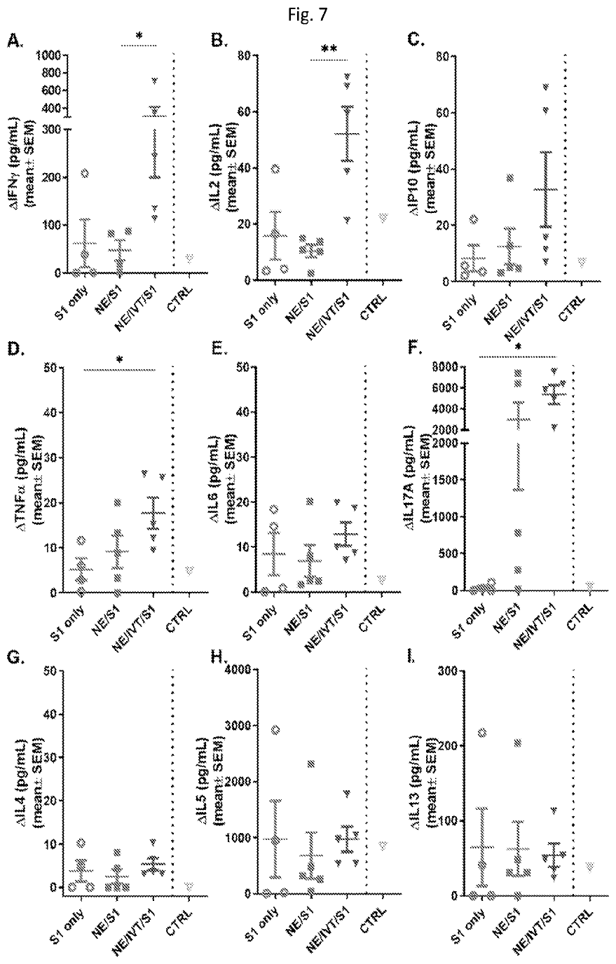

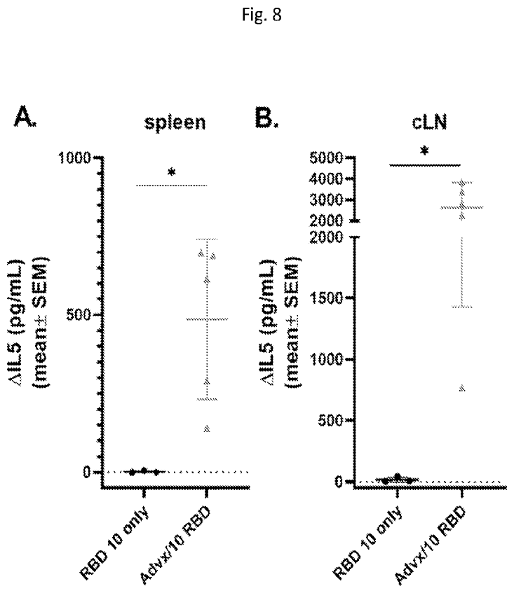

Antigen-specific T-cell recall responses were assessed in splenocytes (FIG. 6) and cells isolated from the draining lymph node (cervical lymph node (cLN)) (FIG. 7) of the immunized mice two weeks after the last immunization (week 10). Splenocytes and cLN were stimulated with recombinant S1 for 72 hours, and cytokine secretion was measured in the cell supernatant by multiplex immunoassay. NE/IVT DI administered through the IN route with S1 induced a heavily magnified TH1 biased response particularly in the draining LN as compared to the NE/S1 single adjuvant group. IFN-7 production in the NE/IVT DI group was increased by an average of 6-fold and by as high as 60-fold, in the spleen, and increased an average of 10-fold and by as high as 230-fold in the cLN as compared to the NE group (FIGS. 6A, 7A). IL-2 production in the NE/IVT DI group was also increased by an average of 2-fold and by as high as 8-fold in the spleen, and increased by an average of 5-fold and by as high as 28-fold in the cLN as compared to the NE group. Additionally, IP-10 and TNF-α were both also enhanced in the spleen and cLN as compared to the NE group. This magnification of TH1 associated cytokines and TNF-α is significant, as co-production of IFN-γ, IL-2, and TNF-α on polyfunctional antigen-specific T-cells has been shown to be the single strongest criteria for predicting vaccine-elicited T-cell mediated protection against viral infection. Upon analysis of TH2 associated cytokines, no significant IL-4 induction was observed in any of the treatment groups, and only minimal levels of IL-13 were observed with NE or NE/IVT DI that were equivalent to that induced by the antigen alone (FIG. 6G, I, 7G, I). NE/IVT DI immunized mice showed slightly higher levels of IL-5 in splenocytes compared to NE alone, however, levels of IL-5 were low overall, being well below that induced by the S1 alone (FIG. 6H). In fact, immunization with NE or NE/IVT DI appeared to reduce the amount of IL-5 and IL-13 induced by the S1 alone (e.g., NE/IVT DI IL-5 was ˜5-10-fold lower than the S1 only group). While IL-5 levels were higher in the cLN, a similar pattern was observed in which NE and NE/IVT DI had similar or reduced levels of IL-5 relative to the S1 alone (FIG. 7H). Interestingly, when IL-5 production after immunization through the IM route was compared with Addavax (MF59), markedly higher levels of IL-5 (>3,000 μg/mL) were produced in the spleen and cLN upon antigen recall evaluation than in the NE or NE/IVT groups (FIG. 8).

In addition to the TH1 response, a pronounced TH17 response—as indicated by the production of IL-17A—was also induced by the NE and enhanced significantly by the NE/IVT DI in the spleen and the cLN. NE/IVT DI enhanced IL-17A production by an average of ˜10-fold in the spleen, and ˜7-fold in the cLN relative to the NE group. A similar cytokine response profile has been observed upon immunization of mice with NE/IVT DI and inactivated influenza virus, including magnified TH1 and TH17 responses. Induction of a TH17 response is unique to the mucosal route of immunization with NE, and was previously demonstrated to be a critical component of NE-mediated protective immunity through the IN route.

### Example 5

This example describes the ability of the NE/IVT to induce broad protective immune responses to SARS-CoV-2 using the receptor binding domain (RBD) of the S protein.

### Materials and Methods for Examples 5-11

### Adjuvants and Antigen

NE was produced by emulsifying cetylpyridinium chloride (CPC) and Tween 80 at a 1:6 (w/w) ratio, with ethanol (200 proof), super refined soybean oil (Croda) and molecular grade water using a high-speed homogenizer as previously described. The emulsion was homogenized to a uniform particle size (d=450-550 nm) and charge (zeta potential=50-55 mV). The sequence and synthesis of IVT DI RNA has previously been described in detail. Briefly, SeV DI RNA was amplified using a 5′ primer with the T7 promoter and a 3′ primer with the hepatitis delta virus genomic ribozyme site followed by the T7 terminator and cloned into a pUC19 plasmid. IVT DI was in vitro transcribed using a HiScribe T7 High Yield RNA synthesis kit (New England Biolabs) followed by DNAse I clean-up with a TURBO DNA-free kit (Thermo-Fisher). IVT DI was then purified with an RNeasy purification kit (Qiagen). The absence of endotoxin was verified by limulus amoebocyte lysate assay (ThermoFisher). Recombinant SARS-CoV-2 receptor binding domain (RBD) (aa319-545) derived from the WT (Wuhan-Hu-1) SARS-CoV-2 isolate with a C-terminal His tag was produced in ExpiCHO cells and purified by the University of Michigan Center for Structural Biology as previously described.

### Cell Lines

Vero E6 cells (ATCC) were maintained in DMEM supplemented with 10% heat inactivated fetal bovine serum (HI FBS) and 1× non-essential amino acids (NEAA). HEK293T cells expressing hACE2 (293T-hACE2) were obtained from BEI resources and maintained in HEK293T medium: DMEM containing 4 mM L-glutamine, 4500 mg/L L-glucose, 1 mM sodium pyruvate and 1500 mg/L sodium bicarbonate, supplemented with 10% HI FBS as previously described.

### Viruses

SARS-CoV-2 clinical isolate USA-WA1/2020 (BEI resources; NR-52281) (referred to as the WT virus), and the B.1.351 variant viruses were propagated by culture in Vero E6 cells as previously described. MA SARS-CoV-2: Mouse-adapted SARS-CoV-2 was obtained by serial passage of the USA-WA1/2020 clinical isolate in mice of different backgrounds over eleven passages, as well as on mACE2 expressing Vero E6 cells as previously described. Briefly, the virus was passaged every two days via IN inoculation with lung homogenate derived supernatants from infected mice. All viral stocks were verified by deep sequencing as described. All work with authentic SARS-CoV-2 viruses were performed in certified BSL3 or ABSL3 facilities in accordance with institutional safety and biosecurity procedures.

### Lentivirus Pseudotyped Virus

Generation of pseudotyped lentiviruses (PSVs) expressing the SARS-CoV-2 spike proteins from WT, B.1.351, B.1.617.2, and B.1.1.529 variants was performed as previously described for the WT PSV. Briefly, lentivirus packaging vectors psPAX2, and a plasmid carrying the envelope protein—full-length SARS-CoV-2 spike protein (aa 738-1254 of the WT spike) containing a C-terminal 19 amino acid deletion to remove the ER retention signal (Invivogen)—were co-transfected with a pGF1-CMV proviral plasmid into 293T cells using standard PEI transfection methods (Polysciences, Inc). Removal of the ER retention signal has been shown to dramatically increase the yield of spike pseudotyped lentivirus. The pGF1-CMV plasmid carries GFP and luciferase reporter genes. Supernatants were collected and pooled after 72 h, pelleted by centrifugation at 13,000 rpm for 4 h at 4° C., and resuspended in DMEM. Viral titers (TU/mL) across variants were determined by measuring PSV transduction of GFP in 293T-hACE2 cells. Harvested lentivirus was stored at −80° C. Neutralization assays with these lentivirus PSVs demonstrate good correlation with authentic virus neutralization assays. Animals

For young mice, 6-8-wk-old female C57Bl/6 mice (Jackson Laboratory) were housed in specific pathogen-free conditions. Mice were acclimated for 2 wks prior to initiation of each study. For aged mice, female C57Bl/6 mice that were 8mo-old at initiation of the study were used. For challenge studies, mice were transferred to ABSL3 facilities 2 d prior to serum transfer and subsequent viral challenge.

### Immunization

For intranasal (IN) immunization, mice were anesthetized under isoflurane using an IMPAC6 precision vaporizer and given 12 μL total (6 μL/nare) of each vaccination mixture. Each group received a total of three immunizations of the same formulations at 4-wk intervals. 10 or 20 μg of recombinant RBD was administered alone or with either 20% NE (w/v), or 20% NE with 0.5 μg of IVT DI in PBS. For intramuscular (IM) immunization, mice were given 50% (v/v) Addavax with 10 μg of RBD in PBS in a total volume of 50 μL. Sera were obtained by saphenous vein bleeding 2 and 4 wks after each immunization, and by cardiac puncture at the end of the experiment at week 10. Bronchial alveolar lavage (BAL) was obtained by lung lavage with 0.8 mL PBS containing protease inhibitors. Spleens and cervical lymph nodes were harvested, processed to single-cell suspensions, and cultured for antigen recall response assessment as previously described. For longevity studies, sera were obtained every 2 wks after the last immunization for 33 weeks, after which the mice were sacrificed for T cell response analysis.

### ELISA

Immunograde 96-well ELISA plates (Midsci) were coated with 100 ng RBD in 50 μL PBS/well overnight at 4° C., and then blocked in 200 μL of 5% non-fat dry milk/PBS for 1 h at 37° C. Sera from immunized mice were serially diluted in PBS/0.1% BSA. Blocking buffer was removed, and diluted sera were added to the wells and incubated for 2 h at 37° C. followed by overnight incubation at 4° C. Plates were washed with PBST (0.05% Tween20), and alkaline phosphatase conjugated secondary antibodies diluted in PBS/0.1% BSA were added (goat-anti-mouse IgG, IgG1, IgG2b, or IgG2c Jackson Immuno Research Laboratories). Plates were incubated at 37° C. for 1 h, washed with PBST, and then developed at RT by addition of of p-nitrophenyl phosphate (pNPP) substrate (Sigma-Aldrich). Absorbance was measured at 405 nm, and titers were determined using a cutoff value defined by the sum of the average absorbance at the lowest dilution of naïve serum and two times the standard deviation.

### Pseudovirus Microneutralization (MNT) Assays

9×10³ 293T-hACE2 cells were seeded overnight on white clear bottom 96-well tissue culture plates in HEK293T medium. To titer PSVs, stocks were serially diluted in HEK293T medium with 16 μg/mL polybrene (Sigma-Aldrich), incubated for 1 h at 37° C., and then added to the 293T-hACE2 cells and incubated at 37° C. for 4 h. Infection media was then replaced with fresh HEK293T medium without polybrene and incubated for an additional 72 h at 37° C. Infection medium was removed, and luciferase activity was measured by addition of 25 μL PBS and 25 μL BrightGlo luminescence reagent by an injection luminometer. Cells were incubated with the BrightGlo reagent for 5 m, after which the luminescence was measured over an integration time of 1 s. A PSV titer for use in neutralization assays across variant PSVs was selected based on the titer of WT PSV which gave >100,000 RLUs above background. For microneutralization assays, 293T-hACE2 cells were seeded overnight. Sera from immunized mice were serially diluted by a factor of three, starting at a dilution of 1:50 in HEK293T medium with 16 μg/mL polybrene (Sigma-Aldrich). 50 μL of diluted sera was added to 50 μL of diluted PSVs (8325 TU/mL), incubated for 1 h at 37° C., and then added to 293T-hACE2 cells for incubation at 37° C. for 4 h. Infection medium was removed and replaced with fresh medium without polybrene and incubated for an additional 72 h at 37° C. Luminescence was measured with BrightGlo reagent for all viral variants besides B.1.6.17.2, for which SteadyGlo reagent was used. Neutralization titers were determined as the dilution at which the luminescence remained below the luminescence of the (virus only control-uninfected control)/2.

### Microneutralization Assays

MNT assays with WT SARS-CoV-2 (2019-nCoV/USA-WA1/2020) was performed in a BSL3 facility as previously described. Briefly, 4×10⁴ Vero E6 cells were seeded per well in a 96-well tissue culture plate overnight. Serum samples were heat-inactivated for 30 m at 56° C. and serially diluted by a factor of three, starting at dilutions of 1:30 in infection medium (DMEM, 2% FBS, 1× non-essential amino acids). Diluted sera were incubated with 250×TCID50 of the WT virus (˜40 PFU) for 1 h at 37° C., and then added to the cells for 48 h at 37° C. Cells were fixed in 4% formaldehyde, washed with PBST (0.1% Tween 20), and permeabilized with 0.1% TritonX100 for 20 m at RT. The plates were washed three times in PBST and blocked in blocking buffer (PBST+5% non-fat milk) for 1 h at RT followed by incubation with a 1:1 mixture of an anti-SARS-CoV-2-nucleoprotein and an anti-SARS-CoV-2-Spike monoclonal antibody (Center for Therapeutic Antibody Development at the Icahn School of Medicine at Mount Sinai) for 1.5 h at RT followed by an HRP-conjugated goat-anti-mouse IgG secondary antibody for 1 h at RT. Plates were washed and developed by addition of 100 μL tetramethyl benzidine and quenched with 50 μL 1M H₂SO₄ prior to measuring the absorbance at 450 nm. Percentage inhibition was calculated against virus only infected controls. The 50% inhibitory dilution (ID50) values were calculated for each sample by least squares fit. Samples with ID50 values lower than the limit of detection (inverse of the lowest dilution=30) were designated as having a titer of 10⁰.

### Antigen Recall Response

T cell antigen recall response was assessed in cell isolates from the spleen and cLN of immunized mice 2 wks after the final immunization (week 10, or week 33 for longevity studies). Methods for splenocyte and cLN lymphocyte preparation were previously described. For antigen recall, isolated cells were plated at a density of 8×10⁵ cells/well and stimulated with 5 μg/well RBD (WT) in T cell media (DMEM, 5% FBS, 2 mM L-glutamine, 1% NEAA, 1 mM sodium pyruvate, 10 mM MOPS, 50 μM 2-mercaptoethanol, 100 IU penicillin, and 100 μg/mL streptomycin), in a total volume of 200 μL for 72 h at 37° C. Secreted cytokines (IFN-γ, IL-2, IP10, IL-4, IL-5, IL-6, IL-13, IL-10, IL-17A, and TNF-α) were measured relative to unstimulated cells in supernatants using a Milliplex MAP Magnetic Mouse Cytokine/Chemokine multiplex immunoassay (EMD Millipore).

### Passive Transfer and Challenge

Equal volumes of serum samples were pooled from all of the mice in each immunization group collected after the second immunization (wk 6), and 50 μL of the pooled serum was passively transferred into each C57Bl/6 naïve mouse through the intraperitoneal route 2 h prior to challenge intranasally under mild ketamine/xylazine sedation with 10⁴ PFU of MA-SARS-CoV-2 in 30 μL. For challenge with B.1.351, equal volumes of serum samples were pooled from all of the mice in each immunization group collected after the third immunization (wk 10), and 110 μL of the pooled serum was passively transferred into each C57Bl/6 naïve mouse through the intraperitoneal route 2 h prior to challenge intranasally under mild ketamine/xylazine sedation with 5×10³ PFU of B.1.351 SARS-CoV-2 in 50 μL. Body weight changes were recorded every 24 h, and mice were sacrificed at 3 d.p.i. Lungs were harvested in 500 μL of PBS, and homogenate was prepared for virus titration by plaque assay as previously described.

### Statistical Analysis

Statistical analyses for acute cytokine production, antibody titers, viral neutralization titers, post-challenge lung pfus, and T cell recall responses was performed with GraphPad Prism 9 (GraphPad Software). Comparisons between treatment groups were performed by Mann-Whitney U test.

### Example 6

Intranasal Immunization with NE and NE/IVT Induces Robust Humoral Immune Responses and Elicits Mucosal Antibody Responses in Both Young and Aged Mice

The immune responses induced by NE and NE/IVT in the context of aging were examined in young (2 months old (m.o.) at initiation, 4.5 m.o. at completion) and aged (8 m.o. at initiation, 10.5 m.o. at completion) mice. For these studies, recombinant, monomeric SARS-CoV-2 spike protein receptor binding domain (RBD) was selected as the test antigen. The RBD was chosen such that any differences could be optimally distinguished, as it has been shown to have low immunogenicity as compared to the full-length spike (S) protein and the S1 subunit which we have previously tested with the NE/IVT combined adjuvant. The RBD contains the region of the S protein necessary for binding to the human ACE2 receptor (hACE2) which is required for viral entry, and thus contains the vast majority of epitopes targeted by neutralizing antibodies. In addition, the RBD contains several of the dominant T cell epitopes identified in convalescent patients.

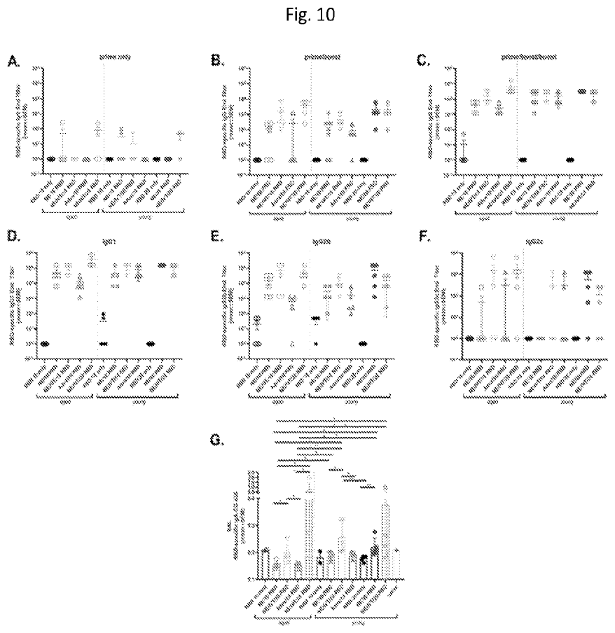

Mice were immunized intranasally with three doses of the same formulation according to a prime/boost/boost schedule with a 4 wk interval between immunizations. Mice were given 10 or 20 μg of RBD with PBS, 20% NE, or 20% NE/0.5 μg IVT (RBD 10 only, RBD 20 only, NE/10 RBD, NE/20 RBD, NE/IVT/10 RBD, NE/IVT/20 RBD, respectively) in a total volume of 12 μL, such that the administered vaccine remained within the nasal cavity. Additional mice immunized intramuscularly (IM) with 10 μg RBD and 50% Addavax, an MF59-like adjuvant, were included for comparison with a licensed parenteral adjuvant (IM Advx/10 RBD). Serum RBD-specific IgG titers were measured two weeks after each immunization at weeks 2, 6, and 10 (FIG. 10A-C). Minimal antigen-specific IgG was detected in aged mice after one immunization. No RBD-specific IgG was detectable in aged treatment groups given RBD 10 only, NE/IVT/10 RBD, or IM Advx/10 RBD. Aged groups given the high dose RBD with the combined NE/IVT adjuvant (NE/IVT/20 RBD) induced low but detectable IgG titers (≤1:250) in 3 out of 5 mice, and the NE/10 RBD group induced detectable titers in 1 out of 5 mice (FIG. 10A). For young mice, low but detectable RBD-specific IgG was detectable in a few mice in the NE/10 RBD, NE/IVT/10 RBD treatment groups, suggesting a potential advantage of the NE and NE/IVT IN adjuvants as compared to the IM Addavax at this early time point. For the high dose RBD groups, no RBD-specific IgG was detected in the RBD alone, or NE/20 RBD groups, whereas low but significant IgG titers were induced in the combined adjuvant NE/IVT/20 RBD group, demonstrating the advantage of the combined NE/IVT adjuvant in both young and aged animals.

IgG titers were increased in all adjuvanted groups after the second immunization (prime/boost) (FIG. 10B). Similar titers were observed between young and aged mice given NE/10 RBD (GMT 43, 23, respectively) and between young and aged mice given NE/IVT/10 RBD (GMT 546, 287, respectively), with slightly higher titers induced by the combined adjuvant as compared to the single NE adjuvant in both age cohorts. Significantly lower titers were observed for both the young and aged IM Advx/10 RBD groups as compared to the IN NE and NE/IVT adjuvanted groups. Importantly, while NE and NE/IVT induced comparable magnitudes of humoral immune responses in young and aged mice, a clear reduction in IgG was observed for the IM Advx/10 RBD in aged mice, with almost all mice having undetectable levels of RBD-specific IgG even after the two immunizations. NE and NE/IVT with the 20 μg RBD dose induced higher titers of IgG than the respective 10 μg RBD groups in both young and aged mice (GMT 1×10³, 2.4×10³, respectively), with the NE/IVT inducing comparable, if not higher IgG in the aged mice compared to the young mice. Similar titers were observed for the single NE and combined NE/IVT IN adjuvants at the higher RBD dose. These results highlight the advantage of these IN adjuvants in terms of inducing stronger humoral responses that are maintained in the context of aging. After the third immunization, (prime/boost/boost) RBD-specific IgG titers further increased for all adjuvanted groups in young and aged mice. After the third immunization, similar high IgG titers were induced in young mice given NE/10 RBD, NE/IVT/10 RBD, and IM Advx/10 RBD (1.2×10⁴, 1.6×10⁴, 1.2×10⁴, respectively). While comparable titers were induced in both young and aged mice for the NE/IVT/10 RBD treatment groups after the final boost, titers for aged mice given NE/10 RBD and IM Advx/10 RBD did not increase by as much as observed in the corresponding young groups, further supporting the advantage of the combined NE/IVT adjuvant. For young groups given 20 μg RBD, similar RBD-specific IgG titers were observed for the NE and NE/IVT groups, which were comparable to what was observed in young mice given the same adjuvants with 10 μg RBD. However, for the NE only groups, the spread in induced IgG titers was reduced, suggesting a more optimal response with the higher antigen dose for the single adjuvant. As was observed after the second immunization, similar or higher titers were seen for the aged mice given NE/IVT/20 RBD as compared to the young animals after the third immunization. While no further increase in IgG was observed in young mice between 10 and 20 μg RBD adjuvanted with NE/IVT, a small increase (half-log) was observed in aged mice with the higher antigen dose.

RBD-specific IgG subclass distributions for IgG1, IgG2b and IgG2c were analyzed at the 10 wk time point (FIG. 10D-F). Subclass analysis revealed a balanced TH1/TH2 profile for the NE and NE/IVT adjuvants, consistent with previous studies. IgG1 titers followed the same relative pattern for each treatment group across age cohorts as total IgG. NE/IVT induced higher IgG1 titers than NE alone in both young and aged mice, and each adjuvant induced similar IgG1 titers in their respective young and aged groups. Robust IgG2b titers were also induced by the NE and NE/IVT adjuvants in young and aged mice. In accordance with previous studies, inclusion of the IVT in the combined NE/IVT adjuvant enhanced IgG2b and 2c titers relative to the single NE adjuvant, in line with the more TH1 skewed profile of the NE/IVT adjuvant. NE/10 RBD and NE/IVT/10 RBD induced higher titers of IgG2b (half log higher) in aged as compared to young mice given the same adjuvants. In contrast, IM Advx/10 RBD induced notably less IgG2b relative to IgG1 as compared to the NE and NE/IVT adjuvanted groups, particularly in aged mice, consistent with the more TH2-polarized properties of this parenteral adjuvant. IgG2c titers induced by NE and NE/IVT were lower than IgG2b, appearing to require a higher antigen dose for optimal induction. However, NE/IVT also appeared to induce higher IgG2c in aged mice, as particularly evident at the higher RBD dose (NE/IVT/20 RBD).

A major advantage to vaccines administered intranasally is the induction of mucosal immune responses. To assess levels of induced mucosal antibodies, bronchial alveolar lavage (BAL) fluid was collected from immunized animals two weeks after the third immunization and levels of RBD-specific IgA were measured (FIG. 10G). At the low antigen dose, no antigen-specific IgA was induced in young or aged mice immunized IM with Advx/10 RBD, consistent with the poor induction of mucosal responses by parenteral vaccination. While no IgA was detected for the RBD 10 only or NE/10 RBD groups in either age group, the combined adjuvant (NE/IVT/10 RBD) induced significantly increased levels of IgA in young mice. However, this effect was diminished in aged mice. At the high RBD dose, while low levels of IgA were observed in young mice given IN NE/20 RBD, these levels were lower than those induced by the NE/IVT/10 RBD. In contrast, the combined adjuvant at the high RBD dose (NE/IVT/20 RBD) induced the highest levels of RBD-specific IgA, and induced similar magnitudes of mucosal responses in both young and aged groups.

### Example 7

### Combined NE/IVT Adjuvant Improves Breadth of Neutralizing Antibodies Against Variants of Concern in Both Young and Aged Mice

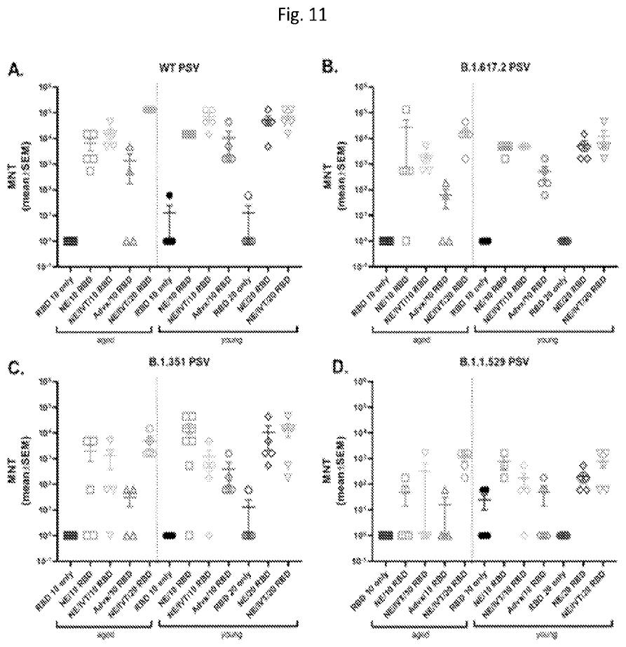

To compare the breadth of virus neutralizing antibodies (nAbs) induced by the NE and NE/IVT in young and aged mice, nAb titers were measured in the serum from immunized mice two weeks after the third immunization using a lentivirus-based pseudovirus (PSV) assay (FIG. 11). Sera was incubated with PSVs expressing the spike protein from the WT, B.1.617.2 (delta), B.1.351 (beta), or B.1.529 (omicron) variants, and entry into hACE2 expressing HEK293T cells was quantified as a function of transduction of a luciferase reporter gene. At the low antigen dose, robust nAb titers were induced by NE and NE/IVT in both young and aged mice against the homologous WT PSV, with the combined adjuvant eliciting marginally higher nAb titers than the single adjuvant in both age groups (FIG. 11A). nAb titers against the WT virus induced by NE/10 RBD and NE/IVT/10 RBD were slightly lower in aged mice as compared to the corresponding young groups, however, titers remained robust in both groups (NE/10 RBD GMT 1.5×10⁴ (young) 3.1×10³ (aged); NE/IVT/10 RBD GMT 5.5×10⁴ (young) 1.17×10⁴ (aged)). IM Advx/10 RBD induced lower nAb titers against the WT virus in young mice than either NE or NE/IVT groups. Moreover, a dramatic reduction in WT nAb titers (97-fold) induced by IM Advx/10 RBD was observed in aged mice, with some mice showing no detectable neutralization despite having similar RBD-specific IgG titers as aged mice given NE/10 RBD (FIG. 10C). These results indicate that the quality of antibodies elicited by NE and NE/IVT is superior to the those elicited by Addavax, particularly in the context of aging. Similarly, while NE/10 RBD and NE/IVT/10 RBD elicited similar RBD-specific IgG titers in young mice, the higher nAb titers observed for the combined adjuvant suggest improved antibody quality. For the WT virus, increasing the RBD dose increased the nAbs induced by the single NE adjuvant to levels equivalent to the combined adjuvant at the low dose (NE/IVT/10 RBD) in young mice. Titers did not further increase in young mice at the higher RBD dose for NE/IVT/20 RBD beyond the already high nAb titers induced by the combined adjuvant at the lower RBD dose (NE/IVT/10 RBD). However, in aged mice, the NE/IVT/20 RBD induced the same high magnitude of nAb titers as induced in the young mice (GMT 5.5×10⁴ (young), 1.3×10⁵ (aged)), better maintaining neutralizing capacity than the other adjuvant/antigen combinations which showed variable degrees of reduction in viral neutralization in the context of aging.

The same relative trends in nAbs between adjuvanted treatment groups were observed against the B.1.617.2 variant as was seen for the WT virus (FIG. 11B). NE and NE/IVT also induced robust nAb titers against the B.1.617.2 variant, inducing GMTs of 3.9×10³ and 4.9×10³, respectively in young mice, and GMTs of 4.6×10² and 1.3×10³, respectively in aged mice, at the low RBD dose. These titers are approximately a 1 log reduction in nAb titers as compared to the WT virus. IM Advx/10 RBD induced lower nAbs against the delta variant in young mice (GMT 2.8×10² (14-fold reduction compared to WT)) than the NE and NE/IVT adjuvants at the same antigen dose and showed significant reduction in nAbs against the delta PSV in aged mice (GMT 1.0×10¹). Multiple studies have observed greater reduction in neutralization of the B.1.351 beta variant by antibodies from mice and humans immunized against the WT spike protein compared to the B.1.617.2 variant. Indeed, a reduction in nAb titers against B.1.351 was observed for the low RBD NE and NE/IVT groups compared to the WT and B.1.617.2 variants in both young and aged mice (FIG. 11C). However, nAbs induced in these groups was significantly higher than the IM Advx/10 RBD group in both age groups. While the NE and NE/IVT groups induced similar B.1.351 nAb titers in both young and aged mice at the low antigen dose, the IM Advx/10 RBD group showed a 19-fold reduction in titer in aged mice relative to young. In contrast, at the higher RBD dose, NE/20 RBD and NE/IVT/20 RBD induced robust cross-neutralizing antibodies in young mice against B.1.351 (GMTs 3.1×10³, 3.9×10³, respectively), which was maintained in aged mice for the combined adjuvant. Finally, serum was evaluated against the B.1.1.529 omicron variant which contains 15 mutations in the RBD relative to the WT virus (FIG. 11D). Significant reduction in neutralization was observed in all groups. While detectable nAbs were observed in NE and NE/IVT groups at the low antigen dose, these titers were low (GMTs<5×10²), and only two out of five mice showed detectable titers in the Advx group. These titers were even further reduced in aged mice for all three groups, and were not significantly different from the RBD alone. In contrast, while low nAbs were elicited against B.1.529 by NE/IVT/20 RBD, titers were maintained in aged mice (GMT 3.5×10² (young), 8.2×10² (aged)). Thus, taken together, these results support induction of broader neutralizing antibody responses by the NE adjuvant in both young and senescent animals, which is further enhanced by the combined NE/IVT adjuvant, especially in aged mice. Further optimization of antigen type (S1 subunit, full-length S protein), antigenic match and dose will likely lead to improved responses to the difficult to neutralize B.1.1.529 variant.

### Example 8

Passively Transferred Antibodies from Young and Aged NE/IVT Immunized Mice Provide Protection from Infection in Naive Mice:

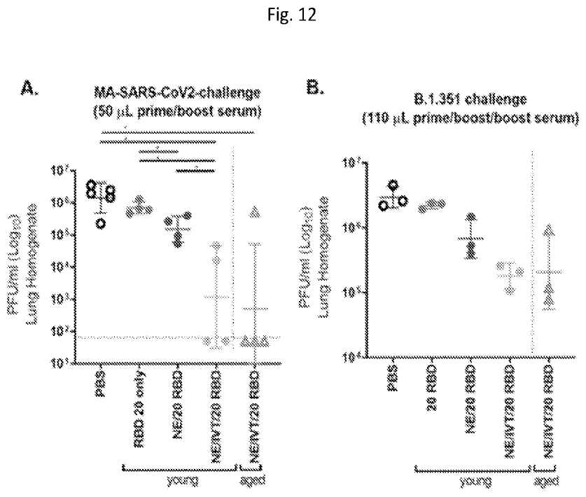

To assess protection afforded by the antibodies induced in young and aged mice by the NE and NE/IVT, sera from immunized mice were transferred into young naïve mice (8 wk old). This allowed evaluation of the antibody component of the vaccine response separate from the cellular immune responses induced by the immunization. Sera from immunized mice in each immunization group were pooled after two immunizations (wk 6), and 50 μL of the pooled serum was transferred IP into each naïve mouse 2 h prior to intranasal challenge with 10⁴ pfu of mouse-adapted SARS-CoV-2 (MA-SARS-CoV-2) (FIG. 12A). This low serum volume was selected for transfer to maximize observable differences between treatment groups. Viral load was assessed in the lung at 3 dpi. The MA virus was adapted from the WT SARS-CoV-2 virus and contains two amino acid substitutions in the S protein as compared to the WT virus, including N501Y and H655Y, and a four aa insertion within the S1 subunit. The N501Y substitution which is shared amongst the B.1.1.7, B.1.351, P.1 and B.1.529 variants allows the MA-CoV2 and these variants to use the mACE2 receptor and directly infect WT mice. No significant reduction in lung viral titer was observed as compared to the mock treatment group (PBS) at the low RBD dose at this early time point after just the prime/boost immunization with NE, NE/IVT, or Advx with serum from either young or aged donor mice. At the higher RBD dose, some potential reduction in viral load was observed for the group which received sera from young, immunized mice immunized IN with 20 RBD alone, but further reduction in lung viral titers was observed for the group receiving serum from young mice immunized with IN NE/20 RBD. However, transferred antibodies alone after two immunizations with NE/20 RBD was insufficient for robust protection against the heterologous MA-CoV2 virus. In contrast, sera from both young and aged mice immunized IN with the combined NE/IVT adjuvant and 20 μg RBD provided significant protection in recipient mice, resulting in a reduction in viral load in all mice and imparting sterilizing immunity in 2 out of 4 and 3 out of 4 mice, respectively. These results clearly demonstrate the advantage of the NE/IVT adjuvant in inducing higher quality, and more cross-protective antibodies. However, protection is not complete after only two immunizations for this serum volume.

Antibody mediated cross-protection against the B.1.351 variant, which is further divergent than the MA-CoV2 from the WT virus, was assessed. For these passive transfer studies, 110 μL of serum pooled from mice given three immunizations (wk 10 sera) was transferred IP into each naïve mouse as above, 2 h prior to IN challenge with 5×10³ pfu of B.1.351 (FIG. 12B). Viral load was assessed in the lung at 3 dpi. No significant reduction in lung viral titer was observed as compared to the mock treatment group (PBS) at the low RBD dose at this early time point after just the prime/boost immunization with NE, NE/IVT, or Advx with serum from either young or aged donor mice. However, a half-log reduction in viral load was observed in mice receiving serum from the young NE/20 RBD immunized group, and a further 1.5 log reduction in viral load was observed for mice receiving sera from either young or aged mice immunized with NE/IVT/20 RBD. Thus, while protection was not complete against the divergent B.1.351 variant, these results clearly demonstrate the benefit of the combined adjuvant and equivalent antibody quality induced by the adjuvant in young and aged mice. These results are consistent with the neutralization titers observed in these immunization groups. Achieving sterilizing immunity may be promoted by a combination of both B and T cell responses. The latter may be particularly the case for the RBD antigen, since it has been shown to be less protective than the S1 subunit or full-length S protein, particularly against heterologous viral variants such as the MA-CoV2.

### Example 9

### Strongly TH1 Polarized Antigen Recall Responses are Induced by NE/IVT Independent of Aging

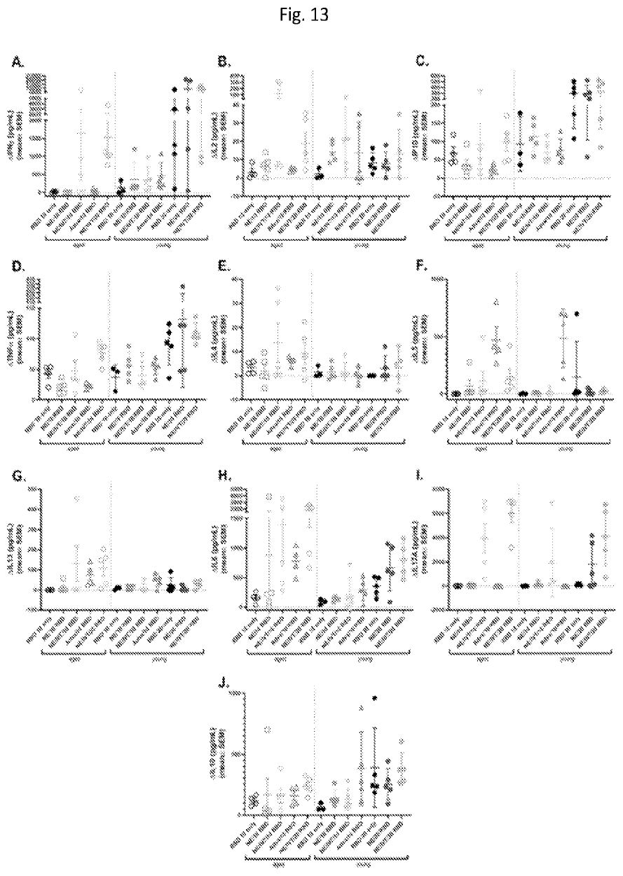

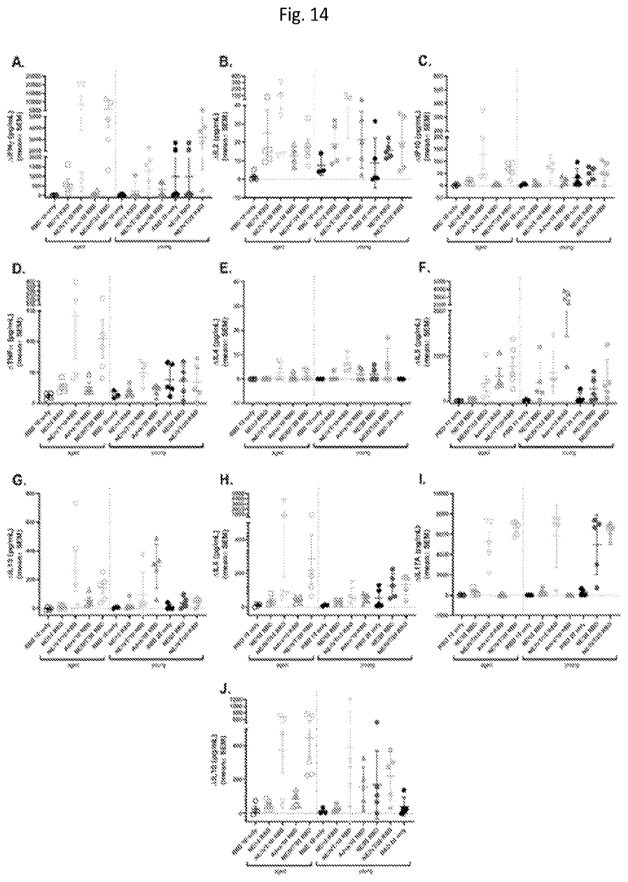

Although significant protection was imparted upon antibody transfer, the importance of robust T cell responses in protection against SARS-CoV-2 has been clearly established especially for protection against severe disease. Furthermore, T cell responses are responsible for maintaining immunity when nAbs wane and for imparting immunity against divergent variants that nAbs fail to effectively neutralize given that T cell epitopes are typically more highly conserved, even across different variants of concern. Accordingly, T cell antigen recall responses were evaluated in the spleen (FIG. 13) and draining lymph nodes (cervical lymph nodes (cLN)) (FIG. 14) of mice immunized with the same RBD/adjuvant formulations as above two weeks after the third immunization (wk 10). Splenocytes and cells isolated from the cLNs were stimulated for 72 h with RBD, and secreted cytokines were measured by multiplex immunoassay and compared to unstimulated cells. The inclusion of IVT DI in the NE/IVT adjuvant dramatically increases the TH1 polarization of the cellular response as compared to NE alone, resulting in synergistic enhancement of TH1-associated cytokine production in both the spleen and cLN. While IFN-γ production in the spleen for the NE/IVT/10 RBD group was only modestly increased in young mice relative to the NE/10 RBD group, IFN-γ was increased by an average of 6-fold in the cLN, as compared to the NE/10 RBD group (FIGS. 13A, 14A). Surprisingly, IFN-γ production was increased by an even higher factor in aged mice immunized with NE/IVT/10 RBD, resulting in an average of 390- and 14-fold increases in the spleen and cLN, respectively as compared to the aged NE/10 RBD group. Similarly high IFN-γ levels were induced at the higher RBD dose with NE/IVT in both young and aged groups. Advx/10 RBD administered through the IM route induced similar levels of IFN-γ production as NE/IVT/10 RBD through the IN route in the spleen for young mice, however Advx induced notably less IFN-γ in the cLN of young mice (reduced by an average of 4-fold relative to NE/IVT/10 RBD). These results are in accordance with the more TH2-bias of the Addavax adjuvant. Importantly, no detectable IFN-γ was induced in aged mice immunized with Advx/10 RBD, suggesting reduced efficacy of the Advx adjuvant in the context of aging. A similar pattern was observed with the other TH1-associated cytokines, IL-2 and IP-10. NE/IVT/10 RBD significantly magnified production of IL-2 compared to NE/10 RBD, maintaining similar levels of induction in both young and aged groups, while Advx showed significantly reduced levels of IL-2 in aged animals (FIGS. 13B, 14B). NE/IVT similarly enhanced IP-10 levels relative to NE alone in aged mice, however differences were small within the spleen and more clearly observable in the cLN (FIGS. 13C, 14C). Thus, for these TH1 cytokines, NE/IVT/10 RBD induced similar or higher levels of cytokine secretion in aged, immunized mice as in young immunized mice, which is in contrast to the consistent reduction observed for these cytokines with the Advx/10 RBD groups in the context of aging. Further, NE/IVT/10 RBD enhanced levels of TNFα production in the cLN as compared to the NE and Advx adjuvants, which induced only low levels (FIG. 14D). NE/IVT/10 RBD elicited higher levels of TNFα in aged mice as compared to young mice, and this effect was even more pronounced for NE/IVT/20 RBD. Such marked enhancement of TH1 associated cytokines along with TNF-α production induced by the combined adjuvant in both young and aged mice is promising, as co-production of IFN-γ, IL-2, and TNF-α on polyfunctional antigen-specific T-cells has been shown to be a strong predictor of effective T-cell mediated protection against viral infection. Moreover, several studies have demonstrated that the reduced capacity of the aging human immune system for antiviral defense is due in part to a progressive shift towards a more TH2 bias as the immune system ages. Thus, an adjuvant capable of inducing strong TH1-biased responses in the context of aging may be able to overcome these deficiencies.

Minimal levels of IL-4 were induced in any of the immunized groups (FIGS. 13E, 14E). While the combined adjuvant induced higher IL-4 in the splenocytes of aged mice than the NE alone or Advx, levels remained low (<38 μg/mL) and were also low in the cLN of both age groups (<12 μg/mL). In contrast to the pattern observed with TH1 cytokines, IM Advx/10 RBD elicited the most robust IL-5 production of all the immunized groups in the spleen which was equal in magnitude between young and aged mice (average 487, 470 μg/mL, respectively), while NE and NE/IVT adjuvants with the same RBD dose elicited minimal IL5 production in the spleen. Advx/10 RBD elicited even greater levels of IL-5 in the cLN of young mice (average 2301 μg/mL), which was 4-fold higher than that elicited by NE/IVT/10 RBD (FIGS. 13F, 14F). However, these levels were significantly lower in aged mice given Advx/10 RBD (485 μg/mL), which was similar to those induced by NE/IVT/10 RBD in aged mice. The high levels of IL-5 produced in the cLN for Advx are notable as the cLN are not the draining lymph nodes for IM immunization. Relatively low levels of IL-13 were induced in the spleen or cLN across all of the young, immunized groups (FIGS. 13G, 14G). However, IL-13 was elevated for young mice immunized with Advx/10 RBD, consistent with the TH2 bias of Addavax. In light of concerns over TH2 associated immunopathology with certain respiratory virus vaccines (vaccine associated enhanced respiratory disease (VAERD)), these levels of IL-13 produced concomitantly with such highly elevated IL-5 may make Addavax a less optimal choice for an adjuvant tailored for SARS-CoV-2. While NE/IVT induced minimal IL13 in the spleen or cLN of young mice, higher levels were observed in aged mice, although these levels remained relatively low and did not occur in the context of highly elevated IL-5. The combined NE/IVT also significantly enhanced IL-6 levels relative to NE alone, inducing high levels of IL-6 particularly at the high RBD dose (FIGS. 13H, 14H). IL-6 levels were also increased in the spleen and cLN of aged mice for all adjuvanted groups as compared to the corresponding young groups. However, minimal induction of IL-6 was observed in the cLN for NE/10 RBD and IM Advx/10 RBD. These results are consistent with prior studies demonstrating the stronger TH1 bias of NE/IVT in the context of a mixed TH1/TH2 profile, and the heavy TH2 bias of Addavax.

Finally, a clear synergistic effect was observed with the NE/IVT in the induction of IL-17A in both the spleen and cLN, resulting in similar levels of induction in aged and young mice (FIGS. 13I, 14I1). IN immunization with NE/IVT/10 RBD elicited averages of 19-fold and 21-fold increases relative to the IN NE/10 RBD group in the spleen and cLN of young mice, respectively, and averages of 75-fold and 18-fold increases in the spleen and cLN of aged mice, respectively. While no significant IL-17A was detectable in the spleen or cLN for the NE alone with 10 μg RBD, increasing the antigen dose to 20 μg resulted in significant levels of induction of IL-17A both in the spleen and cLN, which were then further enhanced by the combined adjuvant at this antigen dose. Parenteral immunization with Advx/10 RBD elicited no IL-17A in either the spleen or cLN. These results are consistent with our previous reports demonstrating that effective TH17 induction is specific to the IN route of immunization with NE. Th17 responses have been demonstrated to be a central component of effective host defense to viral infection, particularly at mucosal surfaces of the respiratory tract. Thus, being able to induce robust TH17 responses along with strong TH1 biased responses particularly in the context of aging with the combined adjuvant provides a powerful tool for driving more potent and tailored immune responses towards SARS-CoV-2. While IL-17A has a critical role in immunoprotective mechanisms, it has also been associated with pathology in certain contexts. However, it has been shown to be protective and non-pathogenic in the context of IL-10 co-production. Indeed, significant levels of IL-10 were elicited in the spleen by NE/IVT/10 RBD in both young and aged groups which was further increased at the higher RBD dose (FIGS. 13J, 14J). IM Advx/10 RBD elicited higher levels of IL10 in the spleen than NE or NE/IVT at the same antigen dose. In contrast, marked enhancement of IL10 was observed with the NE/IVT in the cLN as compared to the NE (or Advx groups), resulting in averages of 16-fold and 8-fold increase relative to the NE alone at the 10 μg RBD dose in young and aged mice, respectively. Similarly high levels were induced by the NE/IVT/20 RBD in young and aged mice.

### Example 10

Immunization with NE and NE/IVT Adjuvants Induce Durable Humoral Immune Responses

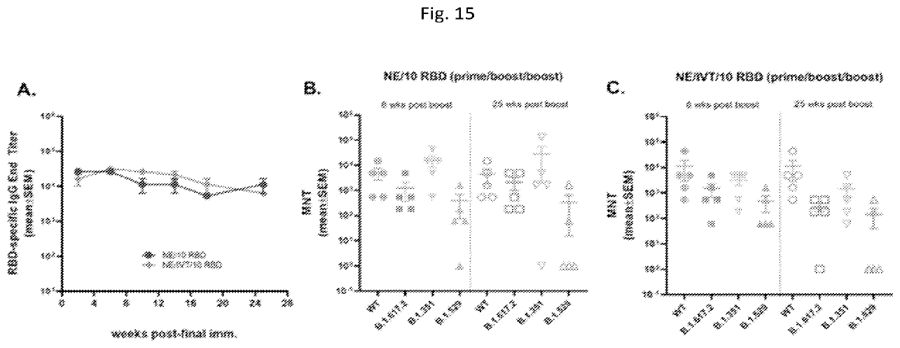

The longevity of the antigen-specific antibody responses induced by NE and the combined NE/IVT adjuvant using the 10 μg RBD antigen dose was examined. 8-wk old mice were given three IN immunizations with either NE/10 RBD, or NE/IVT/10 RBD at a 4-wk interval as above, and serum RBD-specific IgG titers were measured over the course of 25 weeks after the last immunization (up to wk 33 post-initial immunization) (FIG. 15A). RBD-specific total IgG titers remained high and relatively constant in both the NE and NE/IVT groups, showing no or minimal reduction in titer over the course of >6 months after the last immunization. Furthermore, no significant changes in nAb titers were observed for either adjuvanted group over the course >6 months after the final boost immunization for the WT, B.1.617.2, and B.1.351 variants. However, while 4/5 mice from the NE group and 5/5 mice of the NE/IVT group had good levels of neutralization against the B.1.529 variant after 6 weeks, only 2/5 mice for both NE and NE/IVT groups still had detectable nAb titers after >6 months. nAb titers maintained the same relative pattern of efficacy against WT, B.1.617.2, B.1.351, and B.1.529 variants at 6 weeks following the final boost immunization as at >6 months after the final boost, with B.1.529 displaying the lowest cross-neutralization of the variants (FIGS. 15B, C). These results demonstrate that NE and NE/IVT both induce robust and long-lived protective humoral immune responses and support that improving the levels of nAb's initially induced against B.1.529 upon immunization with better antigen selection or design will likely lead to long-lived cross-neutralization against this variant as well.

### Example 11

IN Immunization with RBD Adjuvanted with NE and NE/IVT Induce Long-Lived Antigen Specific Cellular Immune Responses

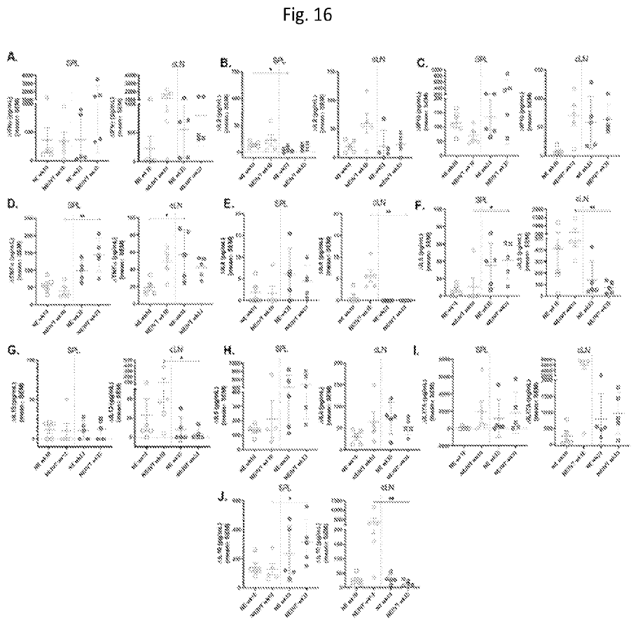

To examine the durability of the T cell responses induced by IN immunization, antigen recall responses were compared in splenocytes and cLN isolates from mice immunized three times IN with NE/10 RBD or NE/IVT/10 RBD at wk10 (2 weeks post-final immunization) or wk 33 (25 weeks post-final immunization). Splenocytes and cLN isolates were stimulated as above with 5 μg of RBD for 72 h, and levels of secreted cytokines were measured in the cell supernatant. Overall, the relative pattern of cytokine production was conserved over six months after the final boost, with the combined NE/IVT adjuvant still demonstrating a greater TH1 bias as compared to the NE alone, inducing higher levels of IFNγ, IL-2, and IP-10 compared to the single adjuvant both at wk 10 and wk 33 in the spleen and cLN (FIGS. 16A-C). The magnitude of the TH1-associated cytokines induced also remained similar, if not increased at the later time point. For example, IFN-γ levels appeared to increase substantially in the spleen in some mice immunized with NE/IVT six months after the final immunization relative to those isolated two weeks after the final immunization (giving an average overall increase of 4-fold, and by as high as 30-fold) (FIG. 16A). Similar increases in TNF-α were observed in the spleen for both NE and NE/IVT immunized mice at six months versus two weeks, as well as in the cLN for the NE alone group. Such maintained (or enhanced) production of T_{H}1-associated cytokines and TNF-α over six months supports the durability of NE/IVT-induced anti-viral T-cell responses.

As with the early T-cell responses, minimal IL-4 was observed upon RBD stimulation at wk 33, with <16 μg/mL produced in the spleen and no detectable IL-4 being observed in the cLN of NE or NE/IVT immunized mice (FIG. 16E). Likewise, while IL-5 appeared modestly increased at wk 33 in the spleen for NE and NE/IVT groups, levels remained low <70 μg/mL, and were markedly reduced for both groups in the cLN at wk 33 relative to wk 10 (FIG. 16F). Similarly, IL-13 remained low in the spleen and were reduced to undetectable levels in the cLN at wk 33 (FIG. 16G). Thus, while T_{H}1-associated cytokines were maintained or enhanced over six months, TH2-associated cytokines were maintained in the spleen but consistently significantly reduced in the cLN. In contrast, IL-6 levels showed a similar pattern as the TH1 cytokines, with IL-6 in the spleen increasing after six months in both NE and NE/IVT groups compared to the wk 10 group, while levels in the cLN were similar at wk10 as wk33 (FIG. 16H). Finally, comparison of IL-17A levels at wk10 and wk33 showed maintenance of the high levels of IL-17A produced in the spleen in response to antigen stimulation, with increased production of IL-17A by the combined adjuvant relative to the single NE adjuvant (FIG. 16I). In contrast, while IL-17A levels in the cLN induced by the NE/IVT at wk 33 were still high and enhanced compared to the NE alone, overall levels in the cLN were notably lower at the late time point (mean 5,839 μg/mL, range 332-7525 μg/mL at wk 10; mean 965 μg/mL range 126-1777 μg/mL at wk 33 for NE/IVT). Significant increases in IL-10 were also observed at the late time point in the spleen, especially for the NE/IVT immunized group, whereas a marked drop in IL-10 production in the cLN was observed at the late time point (FIG. 16J). The analogous pattern in IL-17A and IL-10 could potentially suggest these cytokines are produced by the same non-pathogenic TH17 cells. These results demonstrate that IN immunization with NE/IVT induces long-lived T cell responses, which are potentially more enhanced over time towards a greater TH1/TH17 bias with less TH2 cytokines being induced.

### REFERENCES

All references, including publications, patent applications, and patents, cited herein are hereby incorporated by reference to the same extent as if each reference were individually and specifically indicated to be incorporated by reference and were set forth in its entirety herein.
1. coronavirus.jhu.edu/map.html (accessed 04/27/21).
2. Thanh Le, T.; Andreadakis, Z.; Kumar, A.; Gómez Román, R.; Tollefsen, S.; Saville, M.; Mayhew, S., The COVID-19 vaccine development landscape. Nature reviews. Drug discovery 2020, 19 (5), 305-306.
3. O'Callaghan, K. P.; Blatz, A. M.; Offit, P. A., Developing a SARS-CoV-2 Vaccine at Warp Speed. Jama 2020, 324 (5), 437-438.
4. Corey, L.; Mascola, J. R.; Fauci, A. S.; Collins, F. S., A strategic approach to COVID-19 vaccine R&D. Science 2020, 368 (6494), 948-950.
5. Mascola, J. R.; Graham, B. S.; Fauci, A. S., SARS-CoV-2 Viral Variants-Tackling a Moving Target. Jama 2021.
6. Tegally, H.; Wilkinson, E.; Giovanetti, M.; Iranzadeh, A.; Fonseca, V.; Giandhari, J.; Doolabh, D.; Pillay, S.; San, E. J.; Msomi, N.; Mlisana, K.; von Gottberg, A.; Walaza, S.; Allam, M.; Ismail, A.; Mohale, T.; Glass, A. J.; Engelbrecht, S.; Van Zyl, G.; Preiser, W.; Petruccione, F.; Sigal, A.; Hardie, D.; Marais, G.; Hsiao, M.; Korsman, S.; Davies, M.-A.; Tyers, L.; Mudau, I.; York, D.; Maslo, C.; Goedhals, D.; Abrahams, S.; Laguda-Akingba, O.; Alisoltani-Dehkordi, A.; Godzik, A.; Wibmer, C. K.; Sewell, B. T.; Lourengo, J.; Alcantara, L. C. J.; Pond, S. L. K.; Weaver, S.; Martin, D.; Lessells, R. J.; Bhiman, J. N.; Williamson, C.; de Oliveira, T., Emergence and rapid spread of a new severe acute respiratory syndrome-related coronavirus 2 (SARS-CoV-2) lineage with multiple spike mutations in South Africa. 2020, 2020.12.21.20248640.
7. Supasa, P.; Zhou, D.; Dejnirattisai, W.; Liu, C.; Mentzer, A. J.; Ginn, H. M.; Zhao, Y.; Duyvesteyn, H. M. E.; Nutalai, R.; Tuekprakhon, A.; Wang, B.; Paesen, G. C.; Slon-Campos, J.; López-Camacho, C.; Hallis, B.; Coombes, N.; Bewley, K. R.; Charlton, S.; Walter, T. S.; Barnes, E.; Dunachie, S. J.; Skelly, D.; Lumley, S. F.; Baker, N.; Shaik, I.; Humphries, H. E.; Godwin, K.; Gent, N.; Sienkiewicz, A.; Dold, C.; Levin, R.; Dong, T.; Pollard, A. J.; Knight, J. C.; Klenerman, P.; Crook, D.; Lambe, T.; Clutterbuck, E.; Bibi, S.; Flaxman, A.; Bittaye, M.; Belij-Rammerstorfer, S.; Gilbert, S.; Hall, D. R.; Williams, M. A.; Paterson, N. G.; James, W.; Carroll, M. W.; Fry, E. E.; Mongkolsapaya, J.; Ren, J.; Stuart, D. I.; Screaton, G. R., Reduced neutralization of SARS-CoV-2 B.1.1.7 variant by convalescent and vaccine sera. Cell 2021, 184 (8), 2201-2211.e7.
8. Edara, V. V.; Norwood, C.; Floyd, K.; Lai, L.; Davis-Gardner, M. E.; Hudson, W. H.; Mantus, G.; Nyhoff, L. E.; Adelman, M. W.; Fineman, R.; Patel, S.; Byram, R.; Gomes, D. N.; Michael, G.; Abdullahi, H.; Beydoun, N.; Panganiban, B.; McNair, N.; Hellmeister, K.; Pitts, J.; Winters, J.; Kleinhenz, J.; Usher, J.; O'Keefe, J. B.; Piantadosi, A.; Waggoner, J. J.; Babiker, A.; Stephens, D. S.; Anderson, E. J.; Edupuganti, S.; Rouphael, N.; Ahmed, R.; Wrammert, J.; Suthar, M. S., Reduced binding and neutralization of infection- and vaccine-induced antibodies to the B.1.351 (South African) SARS-CoV-2 variant. bioRxiv 2021.
9. Wang, P.; Casner, R. G.; Nair, M. S.; Wang, M.; Yu, J.; Cerutti, G.; Liu, L.; Kwong, P. D.; Huang, Y.; Shapiro, L.; Ho, D. D., Increased resistance of SARS-CoV-2 variant P.1 to antibody neutralization. Cell Host Microbe 2021.
10. Bian, L.; Gao, F.; Zhang, J.; He, Q.; Mao, Q.; Xu, M.; Liang, Z., Effects of SARS-CoV-2 variants on vaccine efficacy and response strategies. Expert review of vaccines 2021, 1-9.
11. Jangra, S.; Ye, C.; Rathnasinghe, R.; Stadlbauer, D.; Krammer, F.; Simon, V.; Martinez-Sobrido, L.; Garcia-Sastre, A.; Schotsaert, M., SARS-CoV-2 spike E484K mutation reduces antibody neutralisation. The Lancet. Microbe 2021.
12. David, H.; Pengfei, W.; Lihong, L.; Sho, I.; Yang, L.; Yicheng, G.; Maple, W.; Jian, Y.; Baoshan, Z.; Peter, K.; Barney, G.; John, M.; Jennifer, C.; Michael, Y.; Magdalena, S.; Christos, K.; Lawrence, S.; Zizhang, S.; Manoj, N.; Yaoxing, H., Increased Resistance of SARS-CoV-2 Variants B.1.351 and B.1.1.7 to Antibody Neutralization. Nature Portfolio 2021.
13. Johnson, J. Johnson & Johnson announces single-shot Janssen COVID-19 vaccine candidate met primary endpoints in interim analysis of its phase 3 ENSEMBLE trial. https://www.jnj.com/johnson-johnson-announces-single-shot-janssen-covid-19-vaccine-candidate-met-primary-endpoints-in-interim-analysis-of-its-phase-3-ensemble-trial (accessed 02/14/2021).
14. Novavax COVID-19 vaccine demonstrates 89.3% efficacy in UK phase 3 trial. https://ir.novavax.com/news-releases/news-release-details/novavax-covid-19-vaccine-demonstrates-893-efficacy-uk-phase-3 (accessed 02/14/21).
15. Rydyznski Moderbacher, C.; Ramirez, S. I.; Dan, J. M.; Grifoni, A.; Hastie, K. M.; Weiskopf, D.; Belanger, S.; Abbott, R. K.; Kim, C.; Choi, J.; Kato, Y.; Crotty, E. G.; Kim, C.; Rawlings, S. A.; Mateus, J.; Tse, L. P. V.; Frazier, A.; Baric, R.; Peters, B.; Greenbaum, J.; Ollmann Saphire, E.; Smith, D. M.; Sette, A.; Crotty, S., Antigen-Specific Adaptive Immunity to SARS-CoV-2 in Acute COVID-19 and Associations with Age and Disease Severity. Cell 2020, 183 (4), 996-1012.e19.
16. Vabret, N.; Britton, G. J.; Gruber, C.; Hegde, S.; Kim, J.; Kuksin, M.; Levantovsky, R.; Malle, L.; Moreira, A.; Park, M. D.; Pia, L.; Risson, E.; Saffern, M.; Salome, B.; Esai Selvan, M.; Spindler, M. P.; Tan, J.; van der Heide, V.; Gregory, J. K.; Alexandropoulos, K.; Bhardwaj, N.; Brown, B. D.; Greenbaum, B.; Gümüş, Z. H.; Homann, D.; Horowitz, A.; Kamphorst, A. O.; Curotto de Lafaille, M. A.; Mehandru, S.; Merad, M.; Samstein, R. M., Immunology of COVID-19. Current State of the Science. Immunity 2020, 52 (6), 910-941.
17. McMahan, K.; Yu, J.; Mercado, N. B.; Loos, C.; Tostanoski, L. H.; Chandrashekar, A.; Liu, J.; Peter, L.; Atyeo, C.; Zhu, A.; Bondzie, E. A.; Dagotto, G.; Gebre, M. S.; Jacob-Dolan, C.; Li, Z.; Nampanya, F.; Patel, S.; Pessaint, L.; Van Ry, A.; Blade, K.; Yalley-Ogunro, J.; Cabus, M.; Brown, R.; Cook, A.; Teow, E.; Andersen, H.; Lewis, M. G.; Lauffenburger, D. A.; Alter, G.; Barouch, D. H., Correlates of protection against SARS-CoV-2 in rhesus macaques. Nature 2020.
18. Okba, N. M. A.; Müller, M. A.; Li, W.; Wang, C.; GeurtsvanKessel, C. H.; Corman, V. M.; Lamers, M. M.; Sikkema, R. S.; de Bruin, E.; Chandler, F. D.; Yazdanpanah, Y.; Le Hingrat, Q.; Descamps, D.; Houhou-Fidouh, N.; Reusken, C.; Bosch, B. J.; Drosten, C.; Koopmans, M. P. G.; Haagmans, B. L., Severe Acute Respiratory Syndrome Coronavirus 2-Specific Antibody Responses in Coronavirus Disease Patients. Emerging infectious diseases 2020, 26 (7), 1478-1488.
19. Sette, A.; Crotty, S., Adaptive immunity to SARS-CoV-2 and COVID-19. Cell 2021.
20. Grifoni, A.; Sidney, J.; Zhang, Y.; Scheuermann, R. H.; Peters, B.; Sette, A., A Sequence Homology and Bioinformatic Approach Can Predict Candidate Targets for Immune Responses to SARS-CoV-2. Cell Host Microbe 2020, 27 (4), 671-680 e2.
21. Leroux-Roels, G., Prepandemic H5N1 influenza vaccine adjuvanted with AS03: a review of the pre-clinical and clinical data. Expert Opin Biol Ther 2009, 9 (8), 1057-71.
22. Blanco-Melo, D.; Nilsson-Payant, B. E.; Liu, W. C.; Uhl, S.; Hoagland, D.; Moller, R.; Jordan, T. X.; Oishi, K.; Panis, M.; Sachs, D.; Wang, T. T.; Schwartz, R. E.; Lim, J. K.; Albrecht, R. A.; tenOever, B. R., Imbalanced Host Response to SARS-CoV-2 Drives Development of COVID-19. Cell 2020, 181 (5), 1036-1045 e9.
23. Park, A.; Iwasaki, A., Type I and Type III Interferons—Induction, Signaling, Evasion, and Application to Combat COVID-19. Cell Host Microbe 2020, 27 (6), 870-878.
24. Perry, A. K.; Chen, G.; Zheng, D.; Tang, H.; Cheng, G., The host type I interferon response to viral and bacterial infections. Cell research 2005, 15 (6), 407-22.
25. Stanberry, L. R.; Simon, J. K.; Johnson, C.; Robinson, P. L.; Morry, J.; Flack, M. R.; Gracon, S.; Myc, A.; Hamouda, T.; Baker, J. R., Jr., Safety and immunogenicity of a novel nanoemulsion mucosal adjuvant W805EC combined with approved seasonal influenza antigens. Vaccine 2012, 30 (2), 307-16.
26. Safety and Immunogenicity Study of Inactivated Nasal Influenza Vaccine NB-1008 Administered by Sprayer. ClinicalTrials.gov Identifier: NCT01354379 Start date May 2011.
27. A Safety and Immunogenicity of Intranasal Nanoemulsion Adjuvanted Recombinant Anthrax Vaccine in Healthy Adults (IN NE-rPA). ClinicalTrials.gov Identifier: NCT04148118 Start date Jan. 8, 2020.
28. Wong, P. T.; Wang, S. H.; Ciotti, S.; Makidon, P. E.; Smith, D. M.; Fan, Y.; Schuler, C. F. t.; Baker, J. R., Jr., Formulation and characterization of nanoemulsion intranasal adjuvants: effects of surfactant composition on mucoadhesion and immunogenicity. Molecular pharmaceutics 2014, 11 (2), 531-44.
29. Wong, P. T.; Leroueil, P. R.; Smith, D. M.; Ciotti, S.; Bielinska, A. U.; Janczak, K. W.; Mullen, C. H.; Groom, J. V., 2nd; Taylor, E. M.; Passmore, C.; Makidon, P. E.; O'Konek, J. J.; Myc, A.; Hamouda, T.; Baker, J. R., Jr., Formulation, high throughput in vitro screening and in vivo functional characterization of nanoemulsion-based intranasal vaccine adjuvants. Plos One 2015, 10 (5), e0126120.
30. Bielinska, A. U.; Makidon, P. E.; Janczak, K. W.; Blanco, L. P.; Swanson, B.; Smith, D. M.; Pham, T.; Szabo, Z.; Kukowska-Latallo, J. F.; Baker, J. R., Jr., Distinct pathways of humoral and cellular immunity induced with the mucosal administration of a nanoemulsion adjuvant. Journal of immunology (Baltimore, Md.: 1950) 2014, 192 (6), 2722-33.
31. Bielinska, A. U.; Chepurnov, A. A.; Landers, J. J.; Janczak, K. W.; Chepurnova, T. S.; Luker, G. D.; Baker, J. R., Jr., A novel, killed-virus nasal vaccinia virus vaccine. Clin Vaccine Immunol2008, 15 (2), 348-58.
32. Bielinska, A. U.; Janczak, K. W.; Landers, J. J.; Makidon, P.; Sower, L. E.; Peterson, J. W.; Baker, J. R., Jr., Mucosal immunization with a novel nanoemulsion-based recombinant anthrax protective antigen vaccine protects against Bacillus anthracis spore challenge. Infect Immun 2007, 75 (8), 4020-9.
33. Bielinska, A. U.; Janczak, K. W.; Landers, J. J.; Markovitz, D. M.; Montefiori, D. C.; Baker, J. R., Jr., Nasal immunization with a recombinant HIV gp120 and nanoemulsion adjuvant produces Th1 polarized responses and neutralizing antibodies to primary HIV type 1 isolates. AIDS Res Hum Retroviruses 2008, 24 (2), 271-81.
34. Makidon, P. E.; Belyakov, I. M.; Blanco, L. P.; Janczak, K. W.; Landers, J.; Bielinska, A. U.; Groom, J. V., 2nd; Baker, J. R., Jr., Nanoemulsion mucosal adjuvant uniquely activates cytokine production by nasal ciliated epithelium and induces dendritic cell trafficking. Eur J Immunol2012, 42 (8), 2073-86.
35. Myc, A.; Kukowska-Latallo, J. F.; Smith, D. M.; Passmore, C.; Pham, T.; Wong, P.; Bielinska, A. U.; Baker, J. R., Jr., Nanoemulsion nasal adjuvant W(8)(0)5EC induces dendritic cell engulfment of antigen-primed epithelial cells. Vaccine 2013, 31 (7), 1072-9.
36. Martinez-Gil, L.; Goff, P. H.; Hai, R.; Garcia-Sastre, A.; Shaw, M. L.; Palese, P., A Sendai virus-derived RNA agonist of RIG-I as a virus vaccine adjuvant. J Virol 2013, 87 (3), 1290-300.
37. Patel, J. R.; Jain, A.; Chou, Y. Y.; Baum, A.; Ha, T.; Garcia-Sastre, A., ATPase-driven oligomerization of RIG-I on RNA allows optimal activation of type-I interferon. EMBO reports 2013, 14 (9), 780-7.
38. Wong, P. T.; Goff, P. H.; Sun, R. J.; Ruge, M. J.; Ermler, M. E.; Sebring, A.; O'Konek, J. J.; Landers, J. J.; Janczak, K. W.; Sun, W.; Baker, J. R., Jr., Combined Intranasal Nanoemulsion and RIG-I Activating RNA Adjuvants Enhance Mucosal, Humoral, and Cellular Immunity to Influenza Virus. Molecular pharmaceutics 2020.
39. Wang, S. H.; Chen, J.; Smith, D.; Cao, Z.; Acosta, H.; Fan, Y.; Ciotti, S.; Fattom, A.; Baker, J., Jr., A novel combination of intramuscular vaccine adjuvants, nanoemulsion and CpG produces an effective immune response against influenza A virus. Vaccine 2020, 38 (19), 3537-3544.
40. Wang, S. H.; Smith, D.; Cao, Z.; Chen, J.; Acosta, H.; Chichester, J. A.; Yusibov, V.; Streatfield, S. J.; Fattom, A.; Baker, J. R., Jr., Recombinant H5 hemagglutinin adjuvanted with nanoemulsion protects ferrets against pathogenic avian influenza virus challenge. Vaccine 2019, 37 (12), 1591-1600.
41. O'Konek, J. J.; Makidon, P. E.; Landers, J. J.; Cao, Z.; Malinczak, C. A.; Pannu, J.; Sun, J.; Bitko, V.; Ciotti, S.; Hamouda, T.; Wojcinski, Z. W.; Lukacs, N. W.; Fattom, A.; Baker, J. R., Jr., Intranasal nanoemulsion-based inactivated respiratory syncytial virus vaccines protect against viral challenge in cotton rats. Human vaccines & immunotherapeutics 2015, 11 (12), 2904-12.
42. Zhang, J.; Wu, Q.; Liu, Z.; Wang, Q.; Wu, J.; Hu, Y.; Bai, T.; Xie, T.; Huang, M.; Wu, T.; Peng, D.; Huang, W.; Jin, K.; Niu, L.; Guo, W.; Luo, D.; Lei, D.; Wu, Z.; Li, G.; Huang, R.; Lin, Y.; Xie, X.; He, S.; Deng, Y.; Liu, J.; Li, W.; Lu, Z.; Chen, H.; Zeng, T.; Luo, Q.; Li, Y. P.; Wang, Y.; Liu, W.; Qu, X., Spike-specific circulating T follicular helper cell and cross-neutralizing antibody responses in COVID-19-convalescent individuals. Nature microbiology 2021, 6 (1), 51-58.
43. Bielinska, A. U.; Gerber, M.; Blanco, L. P.; Makidon, P. E.; Janczak, K. W.; Beer, M.; Swanson, B.; Baker, J. R., Jr., Induction of Th17 cellular immunity with a novel nanoemulsion adjuvant. Crit Rev Immunol 2010, 30 (2), 189-99.
44. Passmore, C.; Makidon, P. E.; O'Konek, J. J.; Zahn, J. A.; Pannu, J.; Hamouda, T.; Bitko, V.; Myc, A.; Lukacs, N. W.; Fattom, A.; Baker, J. R., Jr., Intranasal immunization with W 80 5EC adjuvanted recombinant RSV rF-ptn enhances clearance of respiratory syncytial virus in a mouse model. Human vaccines & immunotherapeutics 2014, 10 (3), 615-22.
45. Graham, B. S.; Henderson, G. S.; Tang, Y. W.; Lu, X.; Neuzil, K. M.; Colley, D. G., Priming immunization determines T helper cytokine mRNA expression patterns in lungs of mice challenged with respiratory syncytial virus. Journal of immunology (Baltimore, Md.: 1950) 1993, 151 (4), 2032-40.
46. Bolles, M.; Deming, D.; Long, K.; Agnihothram, S.; Whitmore, A.; Ferris, M.; Funkhouser, W.; Gralinski, L.; Totura, A.; Heise, M.; Baric, R. S., A double-inactivated severe acute respiratory syndrome coronavirus vaccine provides incomplete protection in mice and induces increased eosinophilic proinflammatory pulmonary response upon challenge. J Virol 2011, 85 (23), 12201-15.
47. Honda-Okubo, Y.; Barnard, D.; Ong, C. H.; Peng, B. H.; Tseng, C. T.; Petrovsky, N., Severe acute respiratory syndrome-associated coronavirus vaccines formulated with delta inulin adjuvants provide enhanced protection while ameliorating lung eosinophilic immunopathology. J Virol 2015, 89 (6), 2995-3007.
48. Kim, H. W.; Canchola, J. G.; Brandt, C. D.; Pyles, G.; Chanock, R. M.; Jensen, K.; Parrott, R. H., Respiratory syncytial virus disease in infants despite prior administration of antigenic inactivated vaccine. Am J Epidemiol 1969, 89 (4), 422-34.
49. Connors, M.; Giese, N. A.; Kulkarni, A. B.; Firestone, C. Y.; Morse, H. C., 3rd; Murphy, B. R., Enhanced pulmonary histopathology induced by respiratory syncytial virus (RSV) challenge of formalin-inactivated RSV-immunized BALB/c mice is abrogated by depletion of interleukin-4 (IL-4) and IL-10. J Virol 1994, 68 (8), 5321-5.
50. Rathnasinghe, R.; Jangra, S.; Cupic, A.; Martinez-Romero, C.; Mulder, L. C. F.; Kehrer, T.; Yildiz, S.; Choi, A.; Mena, I.; De Vrieze, J.; Aslam, S.; Stadlbauer, D.; Meekins, D. A.; McDowell, C. D.; Balaraman, V.; Richt, J. A.; De Geest, B. G.; Miorin, L.; Krammer, F.; Simon, V.; Garcia-Sastre, A.; Schotsaert, M., The N501Y mutation in SARS-CoV-2 spike leads to morbidity in obese and aged mice and is neutralized by convalescent and post-vaccination human sera. medRxiv 2021.
51. Ou, X.; Liu, Y.; Lei, X.; Li, P.; Mi, D.; Ren, L.; Guo, L.; Guo, R.; Chen, T.; Hu, J.; Xiang, Z.; Mu, Z.; Chen, X.; Chen, J.; Hu, K.; Jin, Q.; Wang, J.; Qian, Z., Characterization of spike glycoprotein of SARS-CoV-2 on virus entry and its immune cross-reactivity with SARS-CoV. Nat Commun 2020, 11 (1), 1620.
52. Dan, J. M.; Mateus, J.; Kato, Y.; Hastie, K. M.; Yu, E. D.; Faliti, C. E.; Grifoni, A.; Ramirez, S. I.; Haupt, S.; Frazier, A.; Nakao, C.; Rayaprolu, V.; Rawlings, S. A.; Peters, B.; Krammer, F.; Simon, V.; Saphire, E. O.; Smith, D. M.; Weiskopf, D.; Sette, A.; Crotty, S., Immunological memory to SARS-CoV-2 assessed for up to 8 months after infection. Science 2021, 371 (6529).
53. Sahin, U.; Muik, A.; Vogler, I.; Derhovanessian, E.; Kranz, L. M.; Vormehr, M.; Quandt, J.; Bidmon, N.; Ulges, A.; Baum, A.; Pascal, K.; Maurus, D.; Brachtendorf, S.; Lorks, V.; Sikorski, J.; Koch, P.; Hilker, R.; Becker, D.; Eller, A.-K.; Grutzner, J.; Tonigold, M.; Boesler, C.; Rosenbaum, C.; Heesen, L.; Kühnle, M.-C.; Poran, A.; Dong, J. Z.; Luxemburger, U.; Kemmer-Bruck, A.; Langer, D.; Bexon, M.; Bolte, S.; Palanche, T.; Schultz, A.; Baumann, S.; Mahiny, A. J.; Boros, G.; Reinholz, J.; Szabó, G. T.; Karikó, K.; Shi, P.-Y.; Fontes-Garfias, C.; Perez, J. L.; Cutler, M.; Cooper, D.; Kyratsous, C. A.; Dormitzer, P. R.; Jansen, K. U.; Tureci, O., BNT162b2 induces SARS-CoV-2-neutralising antibodies and T cells in humans. 2020, 2020.12.09.20245175.
54. Corbett, K. S.; Edwards, D. K.; Leist, S. R.; Abiona, O. M.; Boyoglu-Barnum, S.; Gillespie, R. A.; Himansu, S.; Schafer, A.; Ziwawo, C. T.; DiPiazza, A. T.; Dinnon, K. H.; Elbashir, S. M.; Shaw, C. A.; Woods, A.; Fritch, E. J.; Martinez, D. R.; Bock, K. W.; Minai, M.; Nagata, B. M.; Hutchinson, G. B.; Wu, K.; Henry, C.; Bahl, K.; Garcia-Dominguez, D.; Ma, L.; Renzi, I.; Kong, W. P.; Schmidt, S. D.; Wang, L.; Zhang, Y.; Phung, E.; Chang, L. A.; Loomis, R. J.; Altaras, N. E.; Narayanan, E.; Metkar, M.; Presnyak, V.; Liu, C.; Louder, M. K.; Shi, W.; Leung, K.; Yang, E. S.; West, A.; Gully, K. L.; Stevens, L. J.; Wang, N.; Wrapp, D.; Doria-Rose, N. A.; Stewart-Jones, G.; Bennett, H.; Alvarado, G. S.; Nason, M. C.; Ruckwardt, T. J.; McLellan, J. S.; Denison, M. R.; Chappell, J. D.; Moore, I. N.; Morabito, K. M.; Mascola, J. R.; Baric, R. S.; Carfi, A.; Graham, B. S., SARS-CoV-2 mRNA vaccine design enabled by prototype pathogen preparedness. Nature 2020, 586 (7830), 567-571.
55. Coffman, R. L.; Sher, A.; Seder, R. A., Vaccine adjuvants: putting innate immunity to work. Immunity 2010, 33 (4), 492-503.
56. Seder, R. A.; Darrah, P. A.; Roederer, M., T-cell quality in memory and protection: implications for vaccine design. Nat Rev Immunol 2008, 8 (4), 247-58.
57. Jangra, S.; De Vrieze, J.; Choi, A.; Rathnasinghe, R.; Laghlali, G.; Uvyn, A.; Van Herck, S.; Nuhn, L.; Deswarte, K.; Zhong, Z.; Sanders, N.; Lienenklaus, S.; David, S.; Strohmeier, S.; Amanat, F.; Krammer, F.; Hammad, H.; Lambrecht, B. N.; Coughlan, L.; Garcia-Sastre, A.; de Geest, B.; Schotsaert, M., Sterilizing Immunity against SARS-CoV-2 Infection in Mice by a Single-Shot and Lipid Amphiphile Imidazoquinoline TLR7/8 Agonist-Adjuvanted Recombinant Spike Protein Vaccine. Angewandte Chemie (International ed. in English) 2021.
58. Goff, P. H.; Eggink, D.; Seibert, C. W.; Hai, R.; Martinez-Gil, L.; Krammer, F.; Palese, P., Adjuvants and immunization strategies to induce influenza virus hemagglutinin stalk antibodies. Plos One 2013, 8 (11), e79194.
59. Nachbagauer, R.; Feser, J.; Naficy, A.; Bernstein, D. I.; Guptill, J.; Walter, E. B.; Berlanda-Scorza, F.; Stadlbauer, D.; Wilson, P. C.; Aydillo, T.; Behzadi, M. A.; Bhavsar, D.; Bliss, C.; Capuano, C.; Carreño, J. M.; Chromikova, V.; Claeys, C.; Coughlan, L.; Freyn, A. W.; Gast, C.; Javier, A.; Jiang, K.; Mariottini, C.; McMahon, M.; McNeal, M.; Solórzano, A.; Strohmeier, S.; Sun, W.; Van der Wielen, M.; Innis, B. L.; Garcia-Sastre, A.; Palese, P.; Krammer, F., A chimeric hemagglutinin-based universal influenza virus vaccine approach induces broad and long-lasting immunity in a randomized, placebo-controlled phase I trial. Nat Med 2021, 27 (1), 106-114.
60. Channappanavar, R.; Fett, C.; Zhao, J.; Meyerholz, D. K.; Perlman, S., Virus-specific memory CD8 T cells provide substantial protection from lethal severe acute respiratory syndrome coronavirus infection. J Virol 2014, 88 (19), 11034-44.
61. Hogan, R. J.; Usherwood, E. J.; Zhong, W.; Roberts, A. A.; Dutton, R. W.; Harmsen, A. G.; Woodland, D. L., Activated antigen-specific CD8+ T cells persist in the lungs following recovery from respiratory virus infections. Journal of immunology (Baltimore, Md.: 1950) 2001, 166 (3), 1813-22.
62. Slutter, B.; Pewe, L. L.; Kaech, S. M.; Harty, J. T., Lung airway-surveilling CXCR3(hi) memory CD8(+) T cells are critical for protection against influenza A virus. Immunity 2013, 39 (5), 939-48.
63. Wu, T.; Hu, Y.; Lee, Y. T.; Bouchard, K. R.; Benechet, A.; Khanna, K.; Cauley, L. S., Lung-resident memory CD8 T cells (TRM) are indispensable for optimal cross-protection against pulmonary virus infection. Journal of leukocyte biology 2014, 95 (2), 215-24.
64. Zhao, J.; Zhao, J.; Mangalam, A. K.; Channappanavar, R.; Fett, C.; Meyerholz, D. K.; Agnihothram, S.; Baric, R. S.; David, C. S.; Perlman, S., Airway Memory CD4(+) T Cells Mediate Protective Immunity against Emerging Respiratory Coronaviruses. Immunity 2016, 44 (6), 1379-91.
65. Crotty, S., T Follicular Helper Cell Biology: A Decade of Discovery and Diseases. Immunity 2019, 50 (5), 1132-1148.
66. Dubin, P. J.; Kolls, J. K., Th17 cytokines and mucosal immunity. Immunol Rev 2008, 226, 160-71.
67. Christensen, D.; Mortensen, R.; Rosenkrands, I.; Dietrich, J.; Andersen, P., Vaccine-induced Th17 cells are established as resident memory cells in the lung and promote local IgA responses. Mucosal Immunol 2017, 10 (1), 260-270.
68. Son, Y. M.; Cheon, I. S.; Wu, Y.; Li, C.; Wang, Z.; Gao, X.; Chen, Y.; Takahashi, Y.; Fu, Y. X.; Dent, A. L.; Kaplan, M. H.; Taylor, J. J.; Cui, W.; Sun, J., Tissue-resident CD4(+) T helper cells assist the development of protective respiratory B and CD8(+) T cell memory responses. Sci Immunol 2021, 6 (55).
69. Gauttier, V.; Morello, A.; Girault, I.; Mary, C.; Belarif, L.; Desselle, A.; Wilhelm, E.; Bourquard, T.; Pengam, S.; Teppaz, G.; Thepenier, V.; Biteau, K.; De Barbeyrac, E.; Kiepferlé, D.; Vasseur, B.; Le Flem, F.; Debieuvre, D.; Costantini, D.; Poirier, N., Tissue-resident memory CD8 T-cell responses elicited by a single injection of a multi-target COVID-19 vaccine. 2020, 2020.08.14.240093.
70. Xu, S.; Cao, X., Interleukin-17 and its expanding biological functions. Cell Mol Immunol 2010, 7 (3), 164-74.
71. Farazuddin, M.; Landers, J. J.; Janczak, K. W.; Lindsey, H. K.; Finkelman, F. D.; Baker, J. R., Jr.; O'Konek, J. J., Mucosal Nanoemulsion Allergy Vaccine Suppresses Alarmin Expression and Induces Bystander Suppression of Reactivity to Multiple Food Allergens. Front Immunol 2021, 12, 599296.
72. Bielinska, A. U.; O'Konek, J. J.; Janczak, K. W.; Baker, J. R., Jr., Immunomodulation of TH2 biased immunity with mucosal administration of nanoemulsion adjuvant. Vaccine 2016, 34 (34), 4017-24.
73. Lindell, D. M.; Morris, S. B.; White, M. P.; Kallal, L. E.; Lundy, P. K.; Hamouda, T.; Baker, J. R., Jr.; Lukacs, N. W., A novel inactivated intranasal respiratory syncytial virus vaccine promotes viral clearance without Th2 associated vaccine-enhanced disease. Plos One 2011, 6 (7), e21823.
74. Crawford, K. H. D.; Eguia, R.; Dingens, A. S.; Loes, A. N.; Malone, K. D.; Wolf, C. R.; Chu, H. Y.; Tortorici, M. A.; Veesler, D.; Murphy, M.; Pettie, D.; King, N. P.; Balazs, A. B.; Bloom, J. D., Protocol and Reagents for Pseudotyping Lentiviral Particles with SARS-CoV-2 Spike Protein for Neutralization Assays. Viruses 2020, 12 (5).
75. Rathnasinghe, R.; Strohmeier, S.; Amanat, F.; Gillespie, V. L.; Krammer, F.; Garcia-Sastre, A.; Coughlan, L.; Schotsaert, M.; Uccellini, M. B., Comparison of transgenic and adenovirus hACE2 mouse models for SARS-CoV-2 infection. Emerg Microbes Infect 2020, 9 (1), 2433-2445.

The use of the terms “a” and “an” and “the” and “at least one” and similar referents in the context of describing the disclosure (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

1. An immunogenic composition comprising:
(a) a nanoemulsion;
(b) an agonist of retinoic acid-inducible gene I (RIG-I) and/or an agonist of a toll-like receptor (TLR); and
(c) a coronavirus vaccine.

2. The immunogenic composition of claim 1, wherein the agonist of RIG-I is an RNA agonist.

3. The immunogenic composition of claim 2, wherein the RNA agonist is a defective interfering (DI) RNA of a Sendai virus (SeV) or an influenza virus.

4. The immunogenic composition of claim 1, wherein the agonist of a TLR is an agonist of TLR3

5. The immunogenic composition of claim 4, wherein the TLR3 agonist is a synthetic double-stranded RNA polyriboinosinic polyribocytidylic acid (pIC).

6. The immunogenic composition of any one of claims 1-5, wherein the nanoemulsion comprises:
(a) a poloxamer surfactant or polysorbate surfactant;
(b) an organic solvent;
(c) a halogen containing compound;
(d) oil, and
(e) water.

7. The immunogenic composition of any one of claims 1-6, wherein the coronavirus vaccine is a protein subunit vaccine, a whole virus vaccine, a live-attenuated virus vaccine, an inactivated virus vaccine, an mRNA vaccine, or a pseudotyped virus vaccine.

8. The immunogenic composition of claim 7, wherein the coronavirus vaccine is mRNA-1273 or BNT162b2.

9. The immunogenic composition of claim 7, wherein the coronavirus vaccine comprises the receptor binding domain (RBD) of the S protein.

10. The use of an immunogenic composition of any one of claims 1-9 in the preparation of a medicament.

11. The use of an immunogenic composition of any one of claims 1-9 in the preparation of a medicament for immunizing an animal against a coronavirus.

12. The use according to claim 11, wherein the animal is a human.

13. The use according to any one of claims 10-12, wherein the medicament is delivered by direct injection either intravenously, intramuscularly or subcutaneously.

14. The use according to any one of claims 10-12, wherein the medicament is administered intranasally.

15. The use according to any one of claims 10-14, wherein the medicament generates an immune response specific for a coronavirus.

16. The use according to claim 15, wherein the coronavirus is SARS CoV-2.

17. The use according to claim 15 or claim 16, wherein the immune response comprises generation of coronavirus-specific neutralizing antibodies and/or coronavirus-specific T cell responses.

18. A method of inducing an immune response in a subject, the method comprising administering a therapeutically effective amount of the immunogenic composition of any one of claims 1-9 to the subject.

19. A method of inducing coronavirus-specific neutralizing antibodies and/or coronavirus-specific T cell responses in a subject, which comprises administering a therapeutically effective amount of the immunogenic composition of any one of claims 1-9 to the subject.

20. A method of inducing an immune response against a coronavirus in a subject, which method comprises administering to a subject in need thereof (i) a coronavirus vaccine (ii) a nanoemulsion, (iii) an agonist of retinoic acid-inducible gene I (RIG-I) and/or an agonist of a toll-like receptor (TLR).

21. The method of claim 20, wherein the coronavirus vaccine, the nanoemulsion, the agonist of RIG-I, and/or the agonist of TLR are present in the same composition.

22. The method of claim 20, wherein the coronavirus vaccine is present in a first composition, and the nanoemulsion, agonist of RIG-I, and/or agonist of a TLR are present in a second composition.

23. The method of claim 20, wherein each of the coronavirus vaccine, the nanoemulsion, the agonist of RIG-I, and/or the agonist of a TLR is present in separate compositions.

24. The method of any one of claims 20-23, wherein the coronavirus is coronavirus OC43, coronavirus 229E, coronavirus NL63, coronavirus HKU1, MERS-CoV, SARS-CoV, or SARS-CoV-2 (COVID-19).

25. The method of claim 24, wherein the coronavirus is SARS-CoV-2 (COVID-19).

26. The method of any one of claims 20-25, wherein the nanoemulsion comprises:
(a) a poloxamer surfactant or polysorbate surfactant;
(b) an organic solvent;
(c) a halogen containing compound;
(d) oil, and
(e) water.

27. The method of claim 26, wherein the nanoemulsion comprises:
(a) Tween 80;
(b) ethanol;
(c) cetylpyridinium chloride (CPC);
(d) soybean oil; and
(e) water.

28. The method of any one of claims 20-27, wherein the agonist of RIG-I is an RNA agonist.

29. The method of claim 28, wherein the RNA agonist is a defective interfering (DI) RNA of a Sendai virus (SeV) or an influenza virus.

30. The method of any one of claims 20-27, wherein the agonist of a TLR is an agonist of TLR3.

31. The method of claim 30, wherein the TLR3 agonist is a synthetic double-stranded RNA polyriboinosinic polyribocytidylic acid (pIC).

32. The method of any one of claims 20-31 wherein the coronavirus vaccine is a protein subunit vaccine, a whole virus vaccine, a live-attenuated virus vaccine, an inactivated virus vaccine, an mRNA vaccine, or a pseudotyped virus vaccine.

33. The method of any one of claims 20-32, wherein the composition(s) is/are administered intranasally.

34. The method of any one of claims 20-32, wherein the composition(s) is/are administered parenterally.

35. The method of claim 23, wherein a composition comprising the coronavirus vaccine is administered to the subject intramuscularly, a composition comprising the nanoemulsion is administered to the subject intranasally, and a composition comprising the agonist of RIG-I and/or a composition comprising the agonist of a TLR is administered to the subject intranasally.

36. The method of any one of claims 20-35, which induces a greater neutralizing antibody response against the coronavirus as compared to administration of the coronavirus vaccine alone.

37. The method of any one of claims 20-36, which induces a cellular immune response against the coronavirus.

38. The method of claim 37, wherein the cellular immune response is a Th1-biased immune response.

39. The method of any one of claims 20-38, wherein the immune response prevents the subject from displaying signs or symptoms of coronavirus infection upon subsequent exposure of the subject to the coronavirus.
